(19) United States
(12) Patent Application Publication
(10) Pub. No.: US 2004/0214861 A1
(43) Pub. Date: Oct. 28, 2004

(54) Compositions of a cyclooxygenase-2 selective inhibitors and 5-HT1B1D antagonists for the treatment and prevention of migraine

(75) Inventor: Karen Seibert, St. Louis, MO (US)

Correspondence Address: SENNIGER POWERS LEAVITT AND ROEDEL, ONE METROPOLITAN SQUARE, 16TH FLOOR, ST LOUIS, MO, 63102, US

(73) Assignee: Pharmacia Corporation

(21) Appl. No.: 10/794,041

(22) Filed: Mar. 5, 2004

Related U.S. Application Data
(60) Provisional application No. 60/458,868, filed on Mar. 28, 2003.

(51) Int. Cl. A61K 31/47; A61K 31/382; A61K 31/353

(52) U.S. Cl. 514/314; 514/456; 514/432

(57) ABSTRACT

The present invention provides compositions and methods for the treatment of migraine. More particularly, the invention provides a combination therapy for the treatment of migraine comprising the administration to a subject of a 5-HT_{1B/1D} agonist in combination with a cyclooxygenase-2 selective inhibitor.

### CROSS REFERENCE TO RELATED APPLICATION

This application claims priority from Provisional Application Ser. No. 60/458,868 filed on Mar. 28, 2003, which is hereby incorporated by reference in its entirety.

### FIELD OF THE INVENTION

The present invention provides compositions and methods for the treatment or prevention of migraine. More particularly, the invention is directed toward a combination therapy for the treatment or prevention of migraine comprising the administration to a subject of a 5-HT_{1B/1D} agonist in combination with a cyclooxygenase-2 selective inhibitor.

### BACKGROUND OF THE INVENTION

Migraine is a chronic condition of recurring attacks of transient focal neurologic symptoms (e.g. aura), headache, or both. By definition, the headache is so intense that it interferes with a subject's ability to function and the functioning of other body systems causing associated symptoms. The prevalence of migraine is 15-20% in women and 6-14% in men. Migraine has a complex pathogenesis and is not completely understood. It is known that 5-Hydroxytrypamine, (5-HT) also known as serotonin, is implicated in the pathogenesis of migraine.

5-HT is found in the gastrointestinal tract, the platelets and the brain. It is known that 5-HT levels fluctuate during a migraine attack and that low levels of 5-HT can precipitate a migraine attack. In addition, agents that selectively interact with 5-HT receptor sites in the body are able to alleviate the symptoms of migraine in a large percentage of sufferers.

There are many types of serotonin receptors known as 5-HT₁ to 5-HT₇, where many of the 5-HT receptor types have subtypes, such as 5-HT_{1A} to 5-HT_{1F}. 5-HT₁ receptors are widely distributed in the brain, particularly in intracranial blood vessels and in the brain stem. 5-HT agonists activate 5-HT receptor sites and increase the actions of 5-HT/serotonin at that site. 5-HT antagonists block the 5-HT receptor site and reduce the 5-HT activity at the blocked site.

### SUMMARY OF THE INVENTION

Among the several aspects of the invention is provided a method and a composition for the treatment or prevention of migraine in a subject. The composition comprises a cyclooxygenase-2 selective inhibitor and a 5-HT_{1B/1D} agonist, and the method comprises administering to the subject a cyclooxygenase-2 selective inhibitor and a 5-HT_{1B/1D} agonist.

Other aspects and objects of the invention will be in part and in part pointed out hereinafter.

Abbreviations and Definitions

The term “anti-migraine agent,” unless otherwise indicated herein, includes an agent that is effective in the prevention, amelioration or delay of migraine symptoms.

The term “serotonergic agent,” unless otherwise indicated herein, includes an agent that interacts with any of the serotonin receptor sites in the body of 5HT₁-5HT₇.

The term “serotonin agonist,” unless otherwise indicated herein, includes an agent that activates a serotonin receptor site. For example, the triptans, selective serotonin reuptake inhibitors, dihydroergotamine and the like.

The term “prevention” includes either preventing the onset of a clinically evident migraine altogether or preventing the onset of a preclinically evident stage of a migraine in a subject. This definition includes prophylactic treatment.

The term “inhibition” as used herein means decrease the severity of a migraine as compared to that which would occur in the absence of the application of the present invention.

The phrase “therapeutically-effective” is intended to qualify the amount of each agent which will achieve the goal of improvement in disorder severity and the frequency of incidence over no treatment or treatment of each agent by itself, while avoiding adverse side effects typically associated with alternative therapies.

The term “subject” for purposes of treatment includes a human or animal subject who is susceptible to migraine. The subject can be a domestic livestock species, a laboratory animal species, a zoo animal or a companion animal. In one embodiment, the subject is a mammal. In a preferred embodiment, the mammal is a human being.

The term “cyclooxygenase-2 selective inhibitor” denotes a compound able to inhibit cyclooxygenase-2 without significant inhibition of cyclooxygenase-1. Preferably, it includes compounds that have a cyclooxygenase-2 IC₅₀ of less than about 0.2 micro molar, and also have a selectivity ratio of cyclooxygenase-2 inhibition over cyclooxygenase-1 inhibition of at least 50, and more preferably of at least 100. Even more preferably, the compounds have a cyclooxygenase-1 IC₅₀ of greater than about 1 micro molar, and more preferably of greater than 10 micro molar. Inhibitors of the cyclooxygenase pathway in the metabolism of arachidonic acid used in the present method may inhibit enzyme activity through a variety of mechanisms. By the way of example, and without limitation, the inhibitors used in the methods described herein may block the enzyme activity directly by acting as a substrate for the enzyme.

The term “hydrido” denotes a single hydrogen atom (H). This hydrido radical may be attached, for example, to an oxygen atom to form a hydroxyl radical or two hydrido radicals may be attached to a carbon atom to form a methylene (—CH₂—) radical.

Where used, either alone or within other terms such as “haloalkyl”, “alkylsulfonyl”, “alkoxyalkyl” and “hydroxyalkyl”, the term “alkyl” embraces linear, cyclic or branched radicals having one to about twenty carbon atoms or, preferably, one to about twelve carbon atoms. More preferred alkyl radicals are “lower alkyl” radicals having one to about ten carbon atoms. Most preferred are lower alkyl radicals having one to about six carbon atoms. Examples of such radicals include methyl, ethyl, n-propyl, isopropyl, n-butyl, isobutyl, sec-butyl, tert-butyl, pentyl, iso-amyl, hexyl and the like.

The term “alkenyl” embraces linear or branched radicals having at least one carbon-carbon double bond of two to about twenty carbon atoms or, preferably, two to about twelve carbon atoms. More preferred alkyl radicals are “lower alkenyl” radicals having two to about six carbon atoms. Examples of alkenyl radicals include ethenyl, propenyl, allyl, propenyl, butenyl and 4-methylbutenyl.

The term “alkynyl” denotes linear or branched radicals having two to about twenty carbon atoms or, preferably, two to about twelve carbon atoms. More preferred alkynyl radicals are “lower alkynyl” radicals having two to about ten carbon atoms. Most preferred are lower alkynyl radicals having two to about six carbon atoms. Examples of such radicals include propargyl, butynyl, and the like.

The terms “alkenyl”, “lower alkenyl”, embrace radicals having “cis” and “trans” orientations, or alternatively, “E” and “Z” orientations. The term “cycloalkyl” embraces saturated carbocyclic radicals having three to twelve carbon atoms. More preferred cycloalkyl radicals are “lower cycloalkyl” radicals having three to about eight carbon atoms. Examples of such radicals include cyclopropyl, cyclobutyl, cyclopentyl and cyclohexyl.

The term “cycloalkenyl” embraces partially unsaturated carbocyclic radicals having three to twelve carbon atoms. More preferred cycloalkenyl radicals are “lower cycloalkenyl” radicals having four to about eight carbon atoms. Examples of such radicals include cyclobutenyl, cyclopentenyl, cyclopentadienyl, and cyclohexenyl.

The term “halo” means halogens such as fluorine, chlorine, bromine or iodine.

The term “haloalkyl” embraces radicals wherein any one or more of the alkyl carbon atoms is substituted with halo as defined above. Specifically embraced are monohaloalkyl, dihaloalkyl and polyhaloalkyl radicals. A monohaloalkyl radical, for one example, may have either an iodo, bromo, chloro or fluoro atom within the radical. Dihalo and polyhaloalkyl radicals may have two or more of the same halo atoms or a combination of different halo radicals. “Lower haloalkyl” embraces radicals having 1-6 carbon atoms. Examples of haloalkyl radicals include fluoromethyl, difluoromethyl, trifluoromethyl, chloromethyl, dichloromethyl, trichloromethyl, trichloromethyl, pentafluoroethyl, heptafluoropropyl, difluorochloromethyl, dichlorofluoromethyl, difluoroethyl, difluoropropyl, dichloroethyl and dichloropropyl.

The term “hydroxyalkyl” embraces linear or branched alkyl radicals having one to about ten carbon atoms any one of which may be substituted with one or more hydroxyl radicals. More preferred hydroxyalkyl radicals are “lower hydroxyalkyl” radicals having one to six carbon atoms and one or more hydroxyl radicals. Examples of such radicals include hydroxymethyl, hydroxyethyl, hydroxypropyl, hydroxybutyl and hydroxyhexyl.

The terms “alkoxy” and “alkyloxy” embrace linear or branched oxy-containing radicals each having alkyl portions of one to about ten carbon atoms. More preferred alkoxy radicals are “lower alkoxy” radicals having one to six carbon atoms. Examples of such radicals include methoxy, ethoxy, propoxy, butoxy and tert-butoxy.

The term “alkoxyalkyl” embraces alkyl radicals having one or more alkoxy radicals attached to the alkyl radical, that is, to form monoalkoxyalkyl and dialkoxyalkyl radicals. The “alkoxy” radicals may be further substituted with one or more halo atoms, such as fluoro, chloro or bromo, to provide haloalkoxy radicals. More preferred haloalkoxy radicals are “lower haloalkoxy” radicals having one to six carbon atoms and one or more halo radicals. Examples of such radicals include fluoromethoxy, chloromethoxy, trifluoromethoxy, trifluoroethoxy, fluoroethoxy and fluoropropoxy.

The term “aryl”, alone or in combination, means a carbocyclic aromatic system containing one, two or three rings wherein such rings may be attached together in a pendent manner or may be fused. The term “aryl” embraces aromatic radicals such as phenyl, naphthyl, tetrahydronaphthyl, indane and biphenyl. Aryl moieties may also be substituted at a substitutable position with one or more substituents selected independently from alkyl, alkoxyalkyl, alkylaminoalkyl, carboxyalkyl, alkoxycarbonylalkyl, aminocarbonylalkyl, alkoxy, aralkoxy, hydroxyl, amino, halo, nitro, alkylamino, acyl, cyano, carboxy, aminocarbonyl, alkoxycarbonyl and aralkoxycarbonyl.

The term “heterocyclyl” embraces saturated, partially unsaturated and unsaturated heteroatom-containing ring-shaped radicals, where the heteroatoms may be selected from nitrogen, sulfur and oxygen. Examples of saturated heterocyclyl radicals include saturated 3 to 6-membered heteromonocylic group containing 1 to 4 nitrogen atoms (e.g. pyrrolidinyl, imidazolidinyl, piperidino, piperazinyl, etc.); saturated 3 to 6-membered heteromonocyclic group containing 1 to 2 oxygen atoms and 1 to 3 nitrogen atoms (e.g. morpholinyl, etc.); saturated 3 to 6-membered heteromonocyclic group containing 1 to 2 sulfur atoms and 1 to 3 nitrogen atoms (e.g., thiazolidinyl, etc.). Examples of partially unsaturated heterocyclyl radicals include dihydrothiophene, dihydropyran, dihydrofuran and dihydrothiazole.

The term “heteroaryl” embraces unsaturated heterocyclyl radicals. Examples of unsaturated heterocyclyl radicals, also termed “heteroaryl” radicals include unsaturated 3 to 6 membered heteromonocyclic group containing 1 to 4 nitrogen atoms, for example, pyrrolyl, pyrrolinyl, imidazolyl, pyrazolyl, pyridyl, pyrimidyl, pyrazinyl, pyridazinyl, triazolyl (e.g., 4H-1,2,4-triazolyl, 1H-1,2,3-triazolyl, 2H-1,2,3-triazolyl, etc.) tetrazolyl (e.g. 1H-tetrazolyl, 2H-tetrazolyl, etc.), etc.; unsaturated condensed heterocyclyl group containing 1 to 5 nitrogen atoms, for example, indolyl, isoindolyl, indolizinyl, benzimidazolyl, quinolyl, isoquinolyl, indazolyl, benzotriazolyl, tetrazolopyridazinyl (e.g., tetrazolo[1,5-b]pyridazinyl, etc.), etc.; unsaturated 3 to 6-membered heteromonocyclic group containing an oxygen atom, for example, pyranyl, furyl, etc.; unsaturated 3 to 6-membered heteromonocyclic group containing a sulfur atom, for example, thienyl, etc.; unsaturated 3- to 6-membered heteromonocyclic group containing 1 to 2 oxygen atoms and 1 to 3 nitrogen atoms, for example, oxazolyl, isoxazolyl, oxadiazolyl (e.g., 1,2,4-oxadiazolyl, 1,3,4-oxadiazolyl, 1,2,5-oxadiazolyl, etc.) etc.; unsaturated condensed heterocyclyl group containing 1 to 2 oxygen atoms and 1 to 3 nitrogen atoms (e.g. benzoxazolyl, benzoxadiazolyl, etc.); unsaturated 3 to 6-membered heteromonocyclic group containing 1 to 2 sulfur atoms and 1 to 3 nitrogen atoms, for example, thiazolyl, thiadiazolyl (e.g., 1,2,4-thiadiazolyl, 1,3,4-thiadiazolyl, 1,2,5-thiadiazolyl, etc.) etc.; unsaturated condensed heterocyclyl group containing 1 to 2 sulfur atoms and 1 to 3 nitrogen atoms (e.g., benzothiazolyl, benzothiadiazolyl, etc.) and the like. The term also embraces radicals where heterocyclyl radicals are fused with aryl radicals. Examples of such fused bicyclic radicals include benzofuran, benzothiophene, and the like. Said “heterocyclyl group” may have 1 to 3 substituents such as alkyl, hydroxyl, halo, alkoxy, oxo, amino and alkylamino.

The term “alkylthio” embraces radicals containing a linear or branched alkyl radical, of one to about ten carbon atoms attached to a divalent sulfur atom. More preferred alkylthio radicals are “lower alkylthio” radicals having alkyl radicals of one to six carbon atoms. Examples of such lower alkylthio radicals are methylthio, ethylthio, propylthio, butylthio and hexylthio.

The term “alkylthioalkyl” embraces radicals containing an alkylthio radical attached through the divalent sulfur atom to an alkyl radical of one to about ten carbon atoms. More preferred alkylthioalkyl radicals are “lower alkylthioalkyl” radicals having alkyl radicals of one to six carbon atoms. Examples of such lower alkylthioalkyl radicals include methylthiomethyl.

The term “alkylsulfinyl” embraces radicals containing a linear or branched alkyl radical, of one to ten carbon atoms, attached to a divalent —S(═O)— radical. More preferred alkylsulfinyl radicals are “lower alkylsulfinyl” radicals having alkyl radicals of one to six carbon atoms. Examples of such lower alkylsulfinyl radicals include methylsulfinyl, ethylsulfinyl, butylsulfinyl and hexylsulfinyl.

The term “sulfonyl”, whether used alone or linked to other terms such as alkylsulfonyl, denotes respectively divalent radicals —SO₂—. “Alkylsulfonyl” embraces alkyl radicals attached to a sulfonyl radical, where alkyl is defined as above. More preferred alkylsulfonyl radicals are “lower alkylsulfonyl” radicals having one to six carbon atoms. Examples of such lower alkylsulfonyl radicals include methylsulfonyl, ethylsulfonyl and propylsulfonyl. The “alkylsulfonyl” radicals may be further substituted with one or more halo atoms, such as fluoro, chloro or bromo, to provide haloalkylsulfonyl radicals. The terms “sulfamyl”, “aminosulfonyl” and “sulfonamidyl” denote NH₂O₂S—.

The term “acyl” denotes a radical provided by the residue after removal of hydroxyl from an organic acid. Examples of such acyl radicals include alkanoyl and aroyl radicals. Examples of such lower alkanoyl radicals include formyl, acetyl, propionyl, butyryl, isobutyryl, valeryl, isovaleryl, pivaloyl, hexanoyl, trifluoroacetyl.

The term “carbonyl”, whether used alone or with other terms, such as “alkoxycarbonyl”, denotes —(C═O)—.

The term “aroyl” embraces aryl radicals with a carbonyl radical as defined above. Examples of aroyl include benzoyl, naphthoyl, and the like and the aryl in said aroyl may be additionally substituted.

The terms “carboxy” or “carboxyl”, whether used alone or with other terms, such as “carboxyalkyl”, denotes —CO₂H.

The term “carboxyalkyl” embraces alkyl radicals substituted with a carboxy radical. More preferred are “lower carboxyalkyl” which embrace lower alkyl radicals as defined above, and may be additionally substituted on the alkyl radical with halo. Examples of such lower carboxyalkyl radicals include carboxymethyl, carboxyethyl and carboxypropyl.

The term “alkoxycarbonyl” means a radical containing an alkoxy radical, as defined above, attached via an oxygen atom to a carbonyl radical. More preferred are “lower alkoxycarbonyl” radicals with alkyl portions having 1 to 6 carbons. Examples of such lower alkoxycarbonyl (ester) radicals include substituted or unsubstituted methoxycarbonyl, ethoxycarbonyl, propoxycarbonyl, butoxycarbonyl and hexyloxycarbonyl.

The terms “alkylcarbonyl”, “arylcarbonyl” and “aralkylcarbonyl” include radicals having alkyl, aryl and aralkyl radicals, as defined above, attached to a carbonyl radical. Examples of such radicals include substituted or unsubstituted methylcarbonyl, ethylcarbonyl, phenylcarbonyl and benzylcarbonyl.

The term “aralkyl” embraces aryl-substituted alkyl radicals such as benzyl, diphenylmethyl, triphenylmethyl, phenylethyl, and diphenylethyl. The aryl in said aralkyl may be additionally substituted with halo, alkyl, alkoxy, halkoalkyl and haloalkoxy. The terms benzyl and phenylmethyl are interchangeable.

The term “heterocyclylalkyl” embraces saturated and partially unsaturated heterocyclyl-substituted alkyl radicals, such as pyrrolidinylmethyl, and heteroaryl-substituted alkyl radicals, such as pyridylmethyl, quinolylmethyl, thienylmethyl, furylethyl, and quinolylethyl. The heteroaryl in said heteroaralkyl may be additionally substituted with halo, alkyl, alkoxy, halkoalkyl and haloalkoxy.

The term “aralkoxy” embraces aralkyl radicals attached through an oxygen atom to other radicals.

The term “aralkoxyalkyl” embraces aralkoxy radicals attached through an oxygen atom to an alkyl radical.

The term “aralkylthio” embraces aralkyl radicals attached to a sulfur atom.

The term “aralkylthioalkyl” embraces aralkylthio radicals attached through a sulfur atom to an alkyl radical.

The term “aminoalkyl” embraces alkyl radicals substituted with one or more amino radicals. More preferred are “lower aminoalkyl” radicals. Examples of such radicals include aminomethyl, aminoethyl, and the like.

The term “alkylamino” denotes amino groups that have been substituted with one or two alkyl radicals. Preferred are “lower N-alkylamino” radicals having alkyl portions having 1 to 6 carbon atoms. Suitable lower alkylamino may be mono or dialkylamino such as N-methylamino, N-ethylamino, N,N-dimethylamino, N,N-diethylamino or the like.

The term “arylamino” denotes amino groups, which have been substituted with one or two aryl radicals, such as N-phenylamino. The “arylamino” radicals may be further substituted on the aryl ring portion of the radical.

The term “aralkylamino” embraces aralkyl radicals attached through an amino nitrogen atom to other radicals. The terms “N-arylaminoalkyl” and “N-aryl-N-alkyl-aminoalkyl” denote amino groups which have been substituted with one aryl radical or one aryl and one alkyl radical, respectively, and having the amino group attached to an alkyl radical. Examples of such radicals include N-phenylaminomethyl and N-phenyl-N-methylaminomethyl.

The term “aminocarbonyl” denotes an amide group of the formula —C(═O)NH₂.

The term “alkylaminocarbonyl” denotes an aminocarbonyl group that has been substituted with one or two alkyl radicals on the amino nitrogen atom. Preferred are “N-alkylaminocarbonyl” “N,N-dialkylaminocarbonyl” radicals. More preferred are “lower N-alkylaminocarbonyl” “lower N,N-dialkylaminocarbonyl” radicals with lower alkyl portions as defined above.

The term “alkylaminoalkyl” embraces radicals having one or more alkyl radicals attached to an aminoalkyl radical.

The term “aryloxyalkyl” embraces radicals having an aryl radical attached to an alkyl radical through a divalent oxygen atom.

The term “arylthioalkyl” embraces radicals having an aryl radical attached to an alkyl radical through a divalent sulfur atom.

### DESCRIPTION OF THE PREFERRED EMBODIMENTS

The present invention provides a combination therapy comprising the administration to a subject of a therapeutically effective amount of a COX-2 selective inhibitor in combination with a therapeutically effective amount of a 5-HT_{1B/1D} agonist. The combination therapy is used to treat or prevent migraine, to inhibit inflammation in the vessels, and to treat or prevent disorders associated with migraine. When administered as part of a combination therapy, the COX-2 selective inhibitor together with the 5-HT_{1B/1D} agonist provide enhanced treatment options as compared to administration of either the 5-HT_{1B/1D} agonist or the COX-2 selective inhibitor alone.

Cyclooxygenase-2 Selective Inhibitors

A number of suitable cyclooxygenase-2 selective inhibitors or an isomer, a pharmaceutically acceptable salt, ester, or prodrug thereof may be employed in a composition of the current invention. In one embodiment, the cyclooxygenase-2 selective inhibitor can be, for example, the cyclooxygenase-2 selective inhibitor meloxicam, Formula B-1 (CAS registry number 71125-38-7) or an isomer, a pharmaceutically acceptable salt, ester, or prodrug of a compound having Formula B-1.

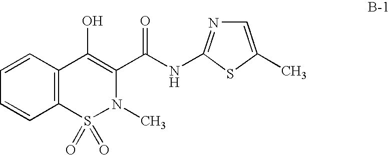

In yet another embodiment, the cyclooxygenase-2 selective inhibitor is the cyclooxygenase-2 selective inhibitor, 6-[[5-(4-chlorobenzoyl)-1,4-dimethyl-1H-pyrrol-2-yl]methyl]-3(2H)-pyridazinone, Formula B-2 (CAS registry number 179382-91-3) or an isomer, a pharmaceutically acceptable salt, ester, or prodrug of a compound having Formula B-2.

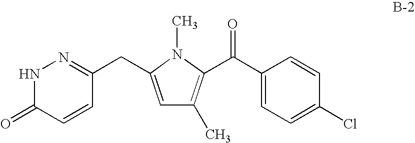

In still another embodiment the cyclooxygenase-2 selective inhibitor is a chromene compound that is a substituted benzopyran or a substituted benzopyran analog, and even more typically, selected from the group consisting of substituted benzothiopyrans, dihydroquinolines, dihydronaphthalenes or a compound having Formula I shown below and possessing, by way of example and not limitation, the structures disclosed in Table 1x. Furthermore, benzopyran cyclooxygenase-2 selective inhibitors useful in the practice of the present methods are described in U.S. Pat. No. 6,034,256 and 6,077,850 herein incorporated by reference in their entirety.

In another embodiment, the cyclooxygenase-2 selective inhibitor is a chromene compound represented by Formula I or an isomer, a pharmaceutically acceptable salt, ester, or prodrug thereof:

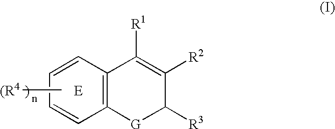

wherein

n is an integer which is 0, 1, 2, 3 or 4;

G is O, S or NR^{a};

R^{a} is alkyl;

R¹ is selected from the group consisting of H and aryl;

R² is selected from the group consisting of carboxyl, aminocarbonyl, alkylsulfonylaminocarbonyl and alkoxycarbonyl;

R³ is selected from the group consisting of haloalkyl, alkyl, aralkyl, cycloalkyl and aryl optionally substituted with one or more radicals selected from alkylthio, nitro and alkylsulfonyl; and

each R⁴ is independently selected from the group consisting of H, halo, alkyl, aralkyl, alkoxy, aryloxy, heteroaryloxy, aralkyloxy, heteroaralkyloxy, haloalkyl, haloalkoxy, alkylamino, arylamino, aralkylamino, heteroarylamino, heteroarylalkylamino, nitro, amino, aminosulfonyl, alkylaminosulfonyl, arylaminosulfonyl, heteroarylaminosulfonyl, aralkylaminosulfonyl, heteroaralkylaminosulfonyl, heterocyclosulfonyl, alkylsulfonyl, hydroxyarylcarbonyl, nitroaryl, optionally substituted aryl, optionally substituted heteroaryl, aralkylcarbonyl, heteroarylcarbonyl, arylcarbonyl, aminocarbonyl, and alkylcarbonyl;

or R⁴ together with the carbon atoms to which it is attached and the remainder of ring E forms a naphthyl radical.

The cyclooxygenase-2 selective inhibitor may also be a compound of Formula (I) or an isomer, a pharmaceutically acceptable salt, ester, or prodrug thereof wherein:

n is an integer which is 0, 1, 2, 3 or 4;

G is O, S or NR^{a};

R¹ is H;

R^{a} is alkyl;

R² is selected from the group consisting of carboxyl, aminocarbonyl, alkylsulfonylaminocarbonyl and alkoxycarbonyl;

R³ is selected from the group consisting of haloalkyl, alkyl, aralkyl, cycloalkyl and aryl, wherein haloalkyl, alkyl, aralkyl, cycloalkyl, and aryl each is independently optionally substituted with one or more radicals selected from the group consisting of alkylthio, nitro and alkylsulfonyl; and

each R⁴ is independently selected from the group consisting of hydrido, halo, alkyl, aralkyl, alkoxy, aryloxy, heteroaryloxy, aralkyloxy, heteroaralkyloxy, haloalkyl, haloalkoxy, alkylamino, arylamino, aralkylamino, heteroarylamino, heteroarylalkylamino, nitro, amino, aminosulfonyl, alkylaminosulfonyl, arylaminosulfonyl, heteroarylaminosulfonyl, aralkylaminosulfonyl, heteroaralkylaminosulfonyl, heterocyclosulfonyl, alkylsulfonyl, optionally substituted aryl, optionally substituted heteroaryl, aralkylcarbonyl, heteroarylcarbonyl, arylcarbonyl, aminocarbonyl, and alkylcarbonyl; or wherein R⁴ together with ring E forms a naphthyl radical.

In a further embodiment, the cyclooxygenase-2 selective inhibitor may also be a compound of Formula (I) or an isomer, a pharmaceutically acceptable salt, ester, or prodrug thereof, wherein:

n is an integer which is 0, 1, 2, 3 or 4;

G is oxygen or sulfur;

R¹ is H;

R² is carboxyl, lower alkyl, lower aralkyl or lower alkoxycarbonyl;

R³ is lower haloalkyl, lower cycloalkyl or phenyl; and

each R⁴ is H, halo, lower alkyl, lower alkoxy, lower haloalkyl, lower haloalkoxy, lower alkylamino, nitro, amino, aminosulfonyl, lower alkylaminosulfonyl, 5-membered heteroarylalkylaminosulfonyl, 6-membered heteroarylalkylaminosulfonyl, lower aralkylaminosulfonyl, 5-membered nitrogen-containing heterocyclosulfonyl, 6-membered-nitrogen containing heterocyclosulfonyl, lower alkylsulfonyl, optionally substituted phenyl, lower aralkylcarbonyl, or lower alkylcarbonyl; or

R⁴ together with the carbon atoms to which it is attached and the remainder of ring E forms a naphthyl radical.

The cyclooxygenase-2 selective inhibitor may also be a compound of Formula (I) or an isomer, a pharmaceutically acceptable salt, ester, or prodrug thereof wherein:

R² is carboxyl;

R³ is lower haloalkyl; and

each R⁴ is H, halo, lower alkyl, lower haloalkyl, lower haloalkoxy, lower alkylamino, amino, aminosulfonyl, lower alkylaminosulfonyl, 5-membered heteroarylalkylaminosulfonyl, 6-membered heteroarylalkylaminosulfonyl, lower aralkylaminosulfonyl, lower alkylsulfonyl, 6-membered nitrogen-containing heterocyclosulfonyl, optionally substituted phenyl, lower aralkylcarbonyl, or lower alkylcarbonyl; or wherein R⁴ together with ring E forms a naphthyl radical.

The cyclooxygenase-2 selective inhibitor may also be a compound of Formula (I) or an isomer, a pharmaceutically acceptable salt, ester, or prodrug thereof wherein:

n is an integer which is 0, 1, 2, 3 or 4;

R³ is fluoromethyl, chloromethyl, dichloromethyl, trichloromethyl, pentafluoroethyl, heptafluoropropyl, difluoroethyl, difluoropropyl, dichloroethyl, dichloropropyl, difluoromethyl, or trifluoromethyl; and

each R⁴ is H, chloro, fluoro, bromo, iodo, methyl, ethyl, isopropyl, tert-butyl, butyl, isobutyl, pentyl, hexyl, methoxy, ethoxy, isopropyloxy, tertbutyloxy, trifluoromethyl, difluoromethyl, trifluoromethoxy, amino, N,N-dimethylamino, N,N-diethylamino, N-phenylmethylaminosulfonyl, N-phenylethylaminosulfonyl, N-(2-furylmethyl)aminosulfonyl, nitro, N,N-dimethylaminosulfonyl, aminosulfonyl, N-methylaminosulfonyl, N-ethylsulfonyl, 2,2-dimethylethylaminosulfonyl, N,N-dimethylaminosulfonyl, N-(2-methylpropyl)aminosulfonyl, N-morpholinosulfonyl, methylsulfonyl, benzylcarbonyl, 2,2-dimethylpropylcarbonyl, phenylacetyl or phenyl; or wherein R⁴ together with the carbon atoms to which it is attached and the remainder of ring E forms a naphthyl radical.

The cyclooxygenase-2 selective inhibitor may also be a compound of Formula (I) or an isomer, a pharmaceutically acceptable salt, ester, or prodrug thereof wherein:

n is an integer which is 0, 1, 2, 3 or 4;

R³ is trifluoromethyl or pentafluoroethyl; and

each R⁴ is independently H, chloro, fluoro, bromo, iodo, methyl, ethyl, isopropyl, tert-butyl, methoxy, trifluoromethyl, trifluoromethoxy, N-phenylmethylaminosulfonyl, N-phenylethylaminosulfonyl, N-(2-furylmethyl)aminosulfonyl, N,N-dimethylaminosulfonyl, N-methylaminosulfonyl, N-(2,2-dimethylethyl)aminosulfonyl, dimethylaminosulfonyl, 2-methylpropylaminosulfonyl, N-morpholinosulfonyl, methylsulfonyl, benzylcarbonyl, or phenyl; or wherein R⁴ together with the carbon atoms to which it is attached and the remainder of ring E forms a naphthyl radical.

In yet another embodiment, the cyclooxygenase-2 selective inhibitor used in connection with the method(s) of the present invention can also be a compound having the structure of Formula (I) or an isomer, a pharmaceutically acceptable salt, ester, or prodrug thereof:

wherein:

n=4;

G is O or S;

R¹ is H;

R² is CO₂H;

R³ is lower haloalkyl;

a first R⁴ corresponding to R⁹ is hydrido or halo;

a second R⁴ corresponding to R¹⁰ is H, halo, lower alkyl, lower haloalkoxy, lower alkoxy, lower aralkylcarbonyl, lower dialkylaminosulfonyl, lower alkylaminosulfonyl, lower aralkylaminosulfonyl, lower heteroaralkylaminosulfonyl, 5-membered nitrogen-containing heterocyclosulfonyl, or 6-membered nitrogen-containing heterocyclosulfonyl;

a third R⁴ corresponding to R¹¹ is H, lower alkyl, halo, lower alkoxy, or aryl; and

a fourth R⁴ corresponding to R¹² is H, halo, lower alkyl, lower alkoxy, and aryl;

wherein Formula (I) is represented by Formula (Ia):

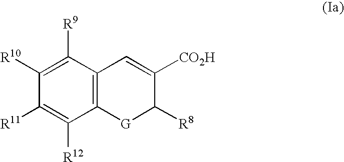

The cyclooxygenase-2 selective inhibitor used in connection with the method(s) of the present invention can also be a compound of having the structure of Formula (Ia) or an isomer, a pharmaceutically acceptable salt, ester, or prodrug thereof wherein:

R⁸ is trifluoromethyl or pentafluoroethyl;

R⁹ is H, chloro, or fluoro;

R¹⁰ is H, chloro, bromo, fluoro, iodo, methyl, tert-butyl, trifluoromethoxy, methoxy, benzylcarbonyl, dimethylaminosulfonyl, isopropylaminosulfonyl, methylaminosulfonyl, benzylaminosulfonyl, phenylethylaminosulfonyl, methylpropylaminosulfonyl, methylsulfonyl, or morpholinosulfonyl;

R¹¹ is H, methyl, ethyl, isopropyl, tert-butyl, chloro, methoxy, diethylamino, or phenyl; and

R¹² is H, chloro, bromo, fluoro, methyl, ethyl, tert-butyl, methoxy, or phenyl.

Examples of exemplary chromene cyclooxygenase-2 selective inhibitors are depicted in Table 1x below.

TABLE 1x
Examples of Chromene Cyclooxygenase-2
Selective Inhibitors as Embodiments

| Compound | |
|---|---|
| Number | Structural Formula |
| B-3 | 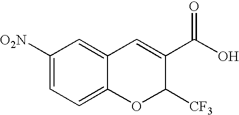 |
| | 6-Nitro-2-trifluoromethyl-2H-1- |
| | benzopyran-3-carboxylic acid |
| B-4 | 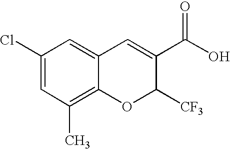 |
| | 6-Chloro-8-methyl-2-trifluoromethyl- |
| | 2H-1-benzopyran-3-carboxylic acid |
| B-5 | 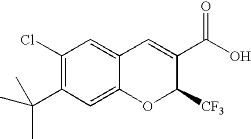 |
| | ((S)-6-Chloro-7-(1,1-dimethylethyl)-2- |
| | (trifromethyl-2H-1-benzopyran-3- |
| | carboxylic aci |
| B-6 | 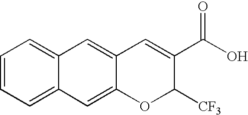 |
| | 2-Trifluoromethyl-2H-naphtho[2,3-b] |
| | pyran-3-carboxylic acid |
| B-7 | 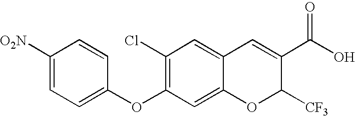 |
| | 6-Chloro-7-(4-nitrophenoxy)-2-(trifluoromethyl) |
| | benzopyran-3-carboxylic acid |
| B-8 | 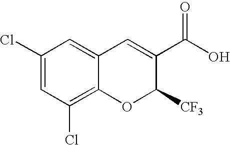 |
| | ((S)-6,8-Dichloro-2-(trifluoromethyl)- |
| | 2H-1-benzopyran-3-carboxylic acid |
| B-9 | 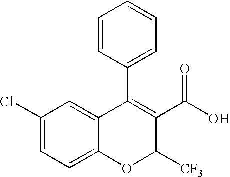 |
| | 6-Chloro-2-(trifluoromethyl)-4-phenyl-2H- |
| | 1-benzopyran-3-carboxylic acid |
| B-10 | 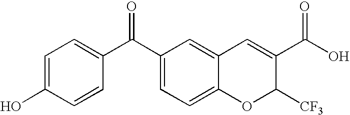 |
| | 6-(4-Hydroxybenzoyl)-2-(trifluoromethyl)- |
| | 2H-1-benzopyran-3-carboxylic acid |
| B-11 | 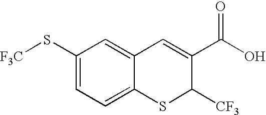 |
| | 2-(Trifluoromethyl)-6-[(trifluoromethyl)th- |
| | 2H-1-benzothiopyran-3-carboxylic acid |
| B-12 | 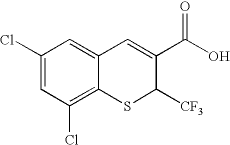 |
| | 6,8-Dichloro-2-trifluoromethyl-2H-1- |
| | benzothiopyran-3-carboxylic acid |
| B-13 | 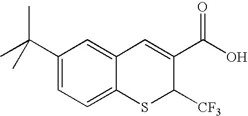 |
| | 6-(1,11-Dimethylethyl)-2-(trifluoromethyl)- |
| | 2H-1-benzothiopyran-3-carboxylic acid |
| B-14 | 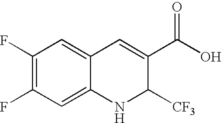 |
| | 6,7-Difluoro-1,2-dihydro-2-(trifluoromethyl)- |
| | 3-quinolinecarboxylic acid |
| B-15 | 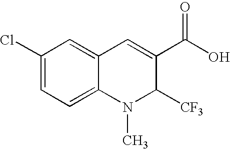 |
| | 6-Chloro-1,2-dihydro-1-methyl-2-(trifluoromethyl)- |
| | 3-quinolinecarboxylic acid |
| B-16 | 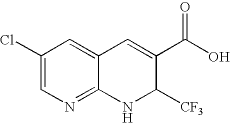 |
| | 6-Chloro-2-(trifluoromethyl)-1,2-dihydro |
| | [1,8]naphthyridine-3-carboxylic acid |
| B-17 | 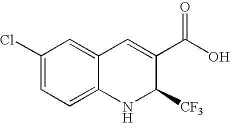 |
| | ((S)-6-Chloro-1,2-dihydro-2--(trifluoromethyl)- |
| | 3-quinolinecarboxylic acid |

In a further embodiment, the cyclooxygenase-2 selective inhibitor is selected from the class of tricyclic cyclooxygenase-2 selective inhibitors represented by the general structure of Formula II or an isomer, a pharmaceutically acceptable salt, ester, or prodrug thereof

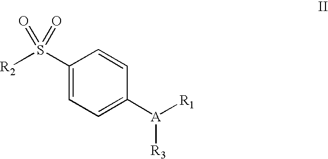

wherein A is selected from the group consisting of partially unsaturated or unsaturated heterocyclyl and partially unsaturated or unsaturated carbocyclic rings;

wherein R₁ is selected from the group consisting of heterocyclyl, cycloalkyl, cycloalkenyl and aryl, wherein R¹ is optionally substituted at a substitutable position with one or more radicals selected from alkyl, haloalkyl, cyano, carboxyl, alkoxycarbonyl, hydroxyl, hydroxyalkyl, haloalkoxy, amino, alkylamino, arylamino, nitro, alkoxyalkyl, alkylsulfinyl, halo, alkoxy and alkylthio;

wherein R₂ is selected from the group consisting of methyl or amino; and

wherein R₃ is selected from the group consisting of a radical selected from H, halo, alkyl, alkenyl, alkynyl, oxo, cyano, carboxyl, cyanoalkyl, heterocyclyloxy, alkyloxy, alkylthio, alkylcarbonyl, cycloalkyl, aryl, haloalkyl, heterocyclyl, cycloalkenyl, aralkyl, heterocyclylalkyl, acyl, alkylthioalkyl, hydroxyalkyl, alkoxycarbonyl, arylcarbonyl, aralkylcarbonyl, aralkenyl, alkoxyalkyl, arylthioalkyl, aryloxyalkyl, aralkylthioalkyl, aralkoxyalkyl, alkoxyaralkoxyalkyl, alkoxycarbonylalkyl, aminocarbonyl, aminocarbonylalkyl, alkylaminocarbonyl, N-arylaminocarbonyl, N-alkyl-N-arylaminocarbonyl, alkylaminocarbonylalkyl, carboxyalkyl, alkylamino, N-arylamino, N-aralkylamino, N-alkyl-N-aralkylamino, N-alkyl-N-arylamino, aminoalkyl, alkylaminoalkyl, N-arylaminoalkyl, N-aralkylaminoalkyl, N-alkyl-N-aralkylaminoalkyl, N-alkyl-N-arylaminoalkyl, aryloxy, aralkoxy, arylthio, aralkylthio, alkylsulfinyl, alkylsulfonyl, aminosulfonyl, alkylaminosulfonyl, N-arylaminosulfonyl, arylsulfonyl, N-alkyl-N-arylaminosulfonyl;

or a pharmaceutically acceptable salt thereof.

In yet another embodiment, the cyclooxygenase-2 selective inhibitor is a compound of forumula II, wherein the compound is not rofecoxib, celecoxib, meloxicam, valdecoxib, etoricoxib (MK-663), JTE 522, L-745337, NS398, tricyclic pyridine compounds disclosed in WO 97/03484 or 6-[[5-(4-chlorobenzoyl)-1,4-dimethyl-1H-pyrrol-2-yl]methyl]-3(2H)-pyridazinone (Compound B-2). The tricyclic pyridine compounds disclosed in WO 97/03484 have the following formula

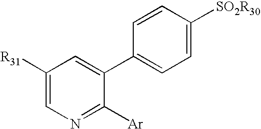

wherein R₃₀ is methyl or amino, Ar is substituted or unsubstituted phenyl or pyridine and R₃₁ is halo, alkyl, cyano, nitro, alkoxy or amido.

Yet another embodiment provides cyclooxygenase-2 selective inhibitors corresponding to formula II wherein A is a ring substituent selected from thienyl, oxazolyl, furyl, pyrrolyl, thiazolyl, imidazolyl, isothiazolyl, isoxazolyl, pyrazolyl, cyclopentenyl, phenyl, and pyridyl; wherein A is optionally substituted with a substituent selected from acyl, halo, hydroxy, lower alkyl, lower haloalkyl, oxo, cyano, nitro, carboxyl, lower alkoxy, aminocarbonyl, lower alkoxycarbonyl, lower carboxyalkyl, lower cyanoalkyl, and lower hydroxyalkyl.

Another embodiment provides cyclooxygenase-2 selective inhibitors corresponding to formula II wherein A is a ring substituent selected from thienyl, pyrrolyl, thiazolyl, imidazolyl, isothiazolyl, cyclopentenyl, and phenyl; wherein A is optionally substituted with a substituent selected from acyl, halo, hydroxy, lower alkyl, lower haloalkyl, oxo, cyano, nitro, carboxyl, lower alkoxy, aminocarbonyl, lower alkoxycarbonyl, lower carboxyalkyl, lower cyanoalkyl, and lower hydroxyalkyl.

Yet another embodiment provides cyclooxygenase-2 selective inhibitors corresponding to formula II wherein A is a ring substituent selected from thienyl, oxazolyl, furyl, pyrrolyl, thiazolyl, imidazolyl, isothiazolyl, isoxazolyl, pyrazolyl, cyclopentenyl, phenyl, and pyridyl; wherein A is optionally substituted with a substituent selected from acyl, halo, hydroxy, lower alkyl, lower haloalkyl, oxo, cyano, nitro, carboxyl, lower alkoxy, aminocarbonyl, lower alkoxycarbonyl, lower carboxyalkyl, lower cyanoalkyl, and lower hydroxyalkyl; provided that when A is pyrazolyl, R³ is other than trifluoromethyl, when A is furanone, R³ is other than hydrido, when A is oxazolyl, R³ is other than methyl, when A is isoxazolyl, R³ is other than methyl and when A is pyridyl, R³ is other than halo, alkyl, cyano, nitro, alkoxy, or amido.

In still another embodiment, the cycloxygenase-2 selective inhibitor is a compound of formula II, provided that when A is pyrazolyl, R³ is other than trifluoromethyl, when A is furanone, R³ is other than hydrido, when A is oxazolyl, R³ is other than methyl, when A is isoxazolyl, R³ is other than methyl and when A is pyridyl, R³ is other than halo, alkyl, cyano, nitro, alkoxy, amido.

In another embodiment, the cyclooxygenase-2 selective inhibitor represented by the above Formula II is selected from the group of compounds, illustrated in Table 2, consisting of celecoxib (B-18; U.S. Pat. No. 5,466,823; CAS No. 169590-42-5), valdecoxib (B-19; U.S. Pat. No. 5,633,272; CAS No. 181695-72-7), deracoxib (B-20; U.S. Pat. No. 5,521,207; CAS No. 169590-41-4), rofecoxib (B-21; CAS No. 162011-90-7), etoricoxib (MK-663; B-22; PCT publication WO 98/03484), JTE-522 (B-23).

TABLE 2x
Examples of Tricyclic Cyclooxygenase-2
Selective Inhibitors as Embodiments

| Compound | |
|---|---|
| Number | Structural Formula |
| B-18 | 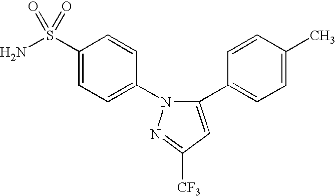 |
| B-19 | 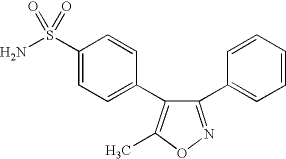 |
| B-20 | 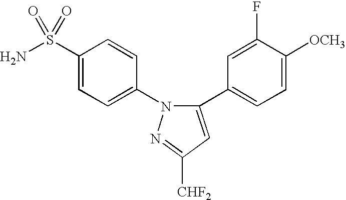 |
| B-21 | 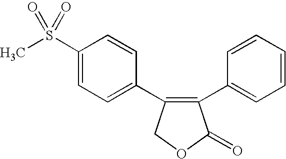 |
| B-22 | 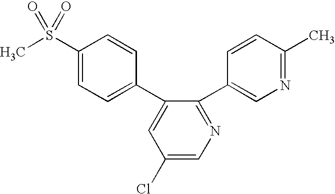 |
| B-23 | 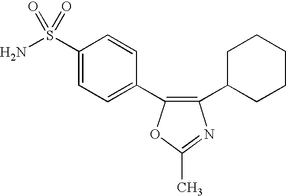 |

In still another embodiment, the cyclooxygenase-2 selective inhibitor is selected from the group consisting of celecoxib, rofecoxib and etoricoxib.

In yet another embodiment, the cyclooxygenase-2 selective inhibitor is parecoxib (B-24, U.S. Pat. No. 5,932,598, CAS No. 198470-84-7), which is a therapeutically effective prodrug of the tricyclic cyclooxygenase-2 selective inhibitor valdecoxib, B-19, may be advantageously employed as a source of a cyclooxygenase inhibitor (U.S. Pat. No. 5,932,598, herein incorporated by reference).

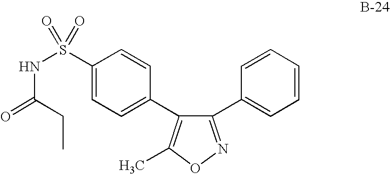

One form of parecoxib is sodium parecoxib.

In another embodiment of the invention, the compound having the formula B-25 or an isomer, a pharmaceutically acceptable salt, ester, or prodrug of a compound having formula B-25 that has been previously described in International Publication number WO 00/24719 (which is herein incorporated by reference) is another tricyclic cyclooxygenase-2 selective inhibitor that may be advantageously employed.

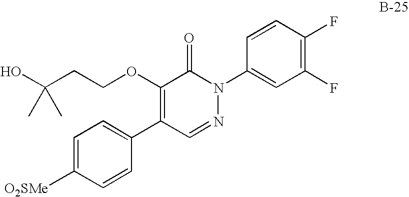

Another cyclooxygenase-2 selective inhibitor that is useful in connection with the method(s) of the present invention is N-(2-cyclohexyloxynitrophenyl)-methane sulfonamide (NS-398) having a structure shown below as B-26, or an isomer, a pharmaceutically acceptable salt, ester, or prodrug of a compound having formula B-26.

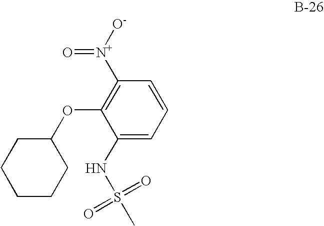

In yet a further embodiment, the cyclooxygenase-2 selective inhibitor thereof used in connection with the method(s) of the present invention can be selected from the class of phenylacetic acid derivative cyclooxygenase-2 selective inhibitors represented by the general structure of Formula (III) or an isomer, a pharmaceutically acceptable salt, ester, or prodrug:

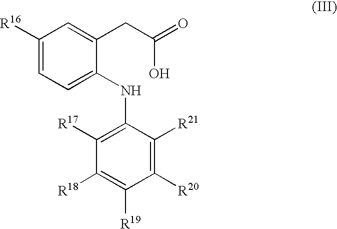

wherein

R¹⁶ is methyl or ethyl;

R¹⁷ is chloro or fluoro;

R¹⁸ is hydrogen or fluoro;

R¹⁹ is hydrogen, fluoro, chloro, methyl, ethyl, methoxy, ethoxy or hydroxy;

R²⁰ is hydrogen or fluoro; and

R²¹ is chloro, fluoro, trifluoromethyl or methyl,

provided that R¹⁷, R¹⁸, R¹⁹ and R²⁰ are not all fluoro when R¹⁶ is ethyl and R¹⁹ is H.

Another phenylacetic acid derivative cyclooxygenase-2 selective inhibitor used in connection with the method(s) of the present invention is a compound that has the designation of COX 189 (lumiracoxib; B-211) and that has the structure shown in Formula (III) or an isomer, a pharmaceutically acceptable salt, ester, or prodrug thereof wherein:

R¹⁶ is ethyl;

R¹⁷ and R¹⁹ are chloro;

R¹⁸ and R²⁰ are hydrogen; and

and R²¹ is methyl.

In yet another embodiment, the cyclooxygenase-2 selective inhibitor is represented by Formula (IV) or an isomer, a pharmaceutically acceptable salt, ester, or prodrug thereof:

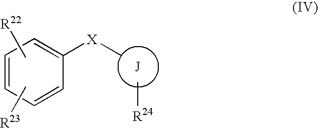

wherein:

X is O or S;

J is a carbocycle or a heterocycle;

R²² is NHSO₂CH₃ or F;

R²³ is H, NO₂, or F; and

R²⁴ is H, NHSO₂CH₃, or (SO₂CH₃)C₆H₄.

According to another embodiment, the cyclooxygenase-2 selective inhibitors used in the present method(s) have the structural Formula (V) or an isomer, a pharmaceutically acceptable salt, ester, or prodrug thereof:

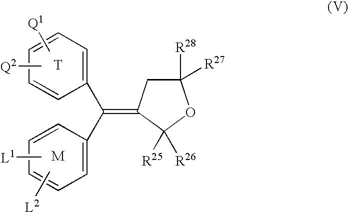

wherein:

T and M independently are phenyl, naphthyl, a radical derived from a heterocycle comprising 5 to 6 members and possessing from 1 to 4 heteroatoms, or a radical derived from a saturated hydrocarbon ring having from 3 to 7 carbon atoms;

Q¹, Q², L¹ or L² are independently hydrogen, halogen, lower alkyl having from 1 to 6 carbon atoms, trifluoromethyl, or lower methoxy having from 1 to 6 carbon atoms; and

at least one of Q¹, Q², L¹ or L² is in the para position and is —S(O)ₙ—R, wherein n is 0, 1, or 2 and R is a lower alkyl radical having 1 to 6 carbon atoms or a lower haloalkyl radical having from 1 to 6 carbon atoms, or an —SO₂NH₂; or,

Q¹ and Q² are methylenedioxy; or

L¹ and L² are methylenedioxy; and

R²⁵, R²⁶, R²⁷, and R²⁸ are independently hydrogen, halogen, lower alkyl radical having from 1 to 6 carbon atoms, lower haloalkyl radical having from 1 to 6 carbon atoms, or an aromatic radical selected from the group consisting of phenyl, naphthyl, thienyl, furyl and pyridyl; or,

R²⁵ and R²⁶ are O; or,

R²⁷ and R²⁸ are O; or,

R²⁵, R²⁶, together with the carbon atom to which they are attached, form a saturated hydrocarbon ring having from 3 to 7 carbon atoms; or,

R²⁷, R²⁸, together with the carbon atom to which they are attached, form a saturated hydrocarbon ring having from 3 to 7 carbon atoms.

In another embodiment, the compounds N-(2-cyclohexyloxynitrophenyl)methane sulfonamide, and (E)-4-[(4-methylphenyl)(tetrahydro-2-oxo-3-furanylidene) methyl]benzenesulfonamide or an isomer, a pharmaceutically acceptable salt, ester, or prodrug thereof having the structure of Formula (V) are employed as cyclooxygenase-2 selective inhibitors.

In a further embodiment, compounds that are useful for the cyclooxygenase-2 selective inhibitor or an isomer, a pharmaceutically acceptable salt, ester, or prodrug thereof used in connection with the method(s) of the present invention, the structures for which are set forth in Table 3x below, include, but are not limited to:

6-chloro-2-trifluoromethyl-2H-1-benzopyran-3-carboxylic acid (B-27);

6-chloro-7-methyl-2-trifluoromethyl-2H-1-benzopyran-3-carboxylic acid (B-28);

8-(1-methylethyl)-2-trifluoromethyl-2H-1-benzopyran-3-carboxylic acid (B-29);

6-chloro-8-(1-methylethyl)-2-trifluoromethyl-2H-1-benzopyran-3-carboxylic acid (B-30);

2-trifluoromethyl-3H-naphtho[2,1-b]pyran-3-carboxylic acid (B-31);

7-(1,1-dimethylethyl)-2-trifluoromethyl-2H-1-benzopyran-3-carboxylic acid (B-32);

6-bromo-2-trifluoromethyl-2H-1-benzopyran-3-carboxylic acid (B-33);

8-chloro-2-trifluoromethyl-2H-1-benzopyran-3-carboxylic acid (B-34);

6-trifluoromethoxy-2-trifluoromethyl-2H-1-benzopyran-3-carboxylic acid (B-35);

5,7-dichloro-2-trifluoromethyl-2H-1-benzopyran-3-carboxylic acid (B-36);

8-phenyl-2-trifluoromethyl-2H-1-benzopyran-3-carboxylic acid (B-37);

7,8-dimethyl-2-trifluoromethyl-2H-1-benzopyran-3-carboxylic acid (B-38);

6,8-bis(dimethylethyl)-2-trifluoromethyl-2H-1-benzopyran-3-carboxylic acid (B-39);

7-(1-methylethyl)-2-trifluoromethyl-2H-1-benzopyran-3-carboxylic acid (B-40);

7-phenyl-2-trifluoromethyl-2H-1-benzopyran-3-carboxylic acid (B-41);

6-chloro-7-ethyl-2-trifluoromethyl-2H-1-benzopyran-3-carboxylic acid (B-42);

6-chloro-8-ethyl-2-trifluoromethyl-2H-1-benzopyran-3-carboxylic acid (B-43);

6-chloro-7-phenyl-2-trifluoromethyl-2H-1-benzopyran-3-carboxylic acid (B-44);

6,7-dichloro-2-trifluoromethyl-2H-1-benzopyran-3-carboxylic acid (B-45);

6,8-dichloro-2-trifluoromethyl-2H-1-benzopyran-3-carboxylic acid (B-46);

6-chloro-8-methyl-2-trifluoromethyl-2H-1-benzopyran-3-carboxylic acid (B-47);

8-chloro-6-methyl-2-trifluoromethyl-2H-1-benzopyran-3-carboxylic acid (B-48)

8-chloro-6-methoxy-2-trifluoromethyl-2H-1-benzopyran-3-carboxylic acid (B-49);

6-bromo-8-chloro-2-trifluoromethyl-2H-1-benzopyran-3-carboxylic acid (B-50);

8-bromo-6-fluoro-2-trifluoromethyl-2H-1-benzopyran-3-carboxylic acid (B-51);

8-bromo-6-methyl-2-trifluoromethyl-2H-1-benzopyran-3-carboxylic acid (B-52);

8-bromo-5-fluoro-2-trifluoromethyl-2H-1-benzopyran-3-carboxylic acid (B-53);

6-chloro-8-fluoro-2-trifluoromethyl-2H-1-benzopyran-3-carboxylic acid (B-54);

6-bromo-8-methoxy-2-trifluoromethyl-2H-1-benzopyran-3-carboxylic acid (B-55);

6-[[(phenylmethyl)amino]sulfonyl]-2-trifluoromethyl-2H-1-benzopyran-3-carboxylic acid (B-56);

6-[(dimethylamino)sulfonyl]-2-trifluoromethyl-2H-1-benzopyran-3-carboxylic acid (B-57);

6-[(methylamino)sulfonyl]-2-trifluoromethyl-2H-1-benzopyran-3-carboxylic acid (B-58);

6-[(4-morpholino)sulfonyl]-2-trifluoromethyl-2H-1-benzopyran-3-carboxylic acid (B-59);

6-[(1,1-dimethylethyl)aminosulfonyl]-2-trifluoromethyl-2H-1-benzopyran-3-carboxylic acid (B-60);

6-[(2-methylpropyl)aminosulfonyl]-2-trifluoromethyl-2H-1-benzopyran-3-carboxylic acid (B-61);

6-methylsulfonyl-2-trifluoromethyl-2H-1-benzopyran-3-carboxylic acid (B-62);

8-chloro-6-[[(phenylmethyl)amino]sulfonyl]-2-trifluoromethyl-2H-1-benzopyran-3-carboxylic acid (B-63);

6-phenylacetyl-2-trifluoromethyl-2H-1-benzopyran-3-carboxylic acid (B-64);

6,8-dibromo-2-trifluoromethyl-2H-1-benzopyran-3-carboxylic acid (B-65);

8-chloro-5,6-dimethyl-2-trifluoromethyl-2H-1-benzopyran-3-carboxylic acid (B-66);

6,8-dichloro-(S)-2-trifluoromethyl-2H-1-benzopyran-3-carboxylic acid (B-67);

6-benzylsulfonyl-2-trifluoromethyl-2H-1-benzopyran-3-carboxylic acid (B-68);

6-[[N-(2-furylmethyl)amino]sulfonyl]-2-trifluoromethyl-2H-1-benzopyran-3-carboxylic acid (B-69);

6-[[N-(2-phenylethyl)amino]sulfonyl]-2-trifluoromethyl-2H-1-benzopyran-3-carboxylic acid (B-70);

6-iodo-2-trifluoromethyl-2H-1-benzopyran-3-carboxylic acid (B-71);

7-(1,1-dimethylethyl)-2-pentafluoroethyl-2H-1-benzopyran-3-carboxylic acid (B-72);

6-chloro-2-trifluoromethyl-2H-1-benzothiopyran-3-carboxylic acid (B-73);

3-[(3-Chloro-phenyl)-(4-methanesulfonyl-phenyl)-methylene]-dihydro-furan-2-one or BMS-347070 (B-74);

8-acetyl-3-(4-fluorophenyl)-2-(4-methylsulfonyl)phenyl-imidazo(1,2-a)pyridine (B-75);

5,5-dimethyl-4-(4-methylsulfonyl)phenyl-3-phenyl-2-(5H)-furanone (B-76);

5-(4-fluorophenyl)-1-[4-(methylsulfonyl)phenyl]-3-(trifluoromethyl)pyrazole (B-77);

4-(4-fluorophenyl)-5-[4-(methylsulfonyl)phenyl]-1-phenyl-3-(trifluoromethyl)pyrazole (B-78);

4-(5-(4-chlorophenyl)-3-(4-methoxyphenyl)-1H-pyrazol-1-yl)benzenesulfonamide (B-79);

4-(3,5-bis(4-methylphenyl)-1H-pyrazol-1-yl)benzenesulfonamide (B-80);

4-(5-(4-chlorophenyl)-3-phenyl-1H-pyrazol-1-yl)benzenesulfonamide (B-81);

4-(3,5-bis(4-methoxyphenyl)-1H-pyrazol-1-yl)benzenesulfonamide (B-82);

4-(5-(4-chlorophenyl)-3-(4-methylphenyl)-1H-pyrazol-1-yl)benzenesulfonamide (B-83);

4-(5-(4-chlorophenyl)-3-(4-nitrophenyl)-1H-pyrazol-1-yl)benzenesulfonamide (B-84);

4-(5-(4-chlorophenyl)-3-(5-chloro-2-thienyl)-1H-pyrazol-1-yl)benzenesulfonamide (B-85);

4-(4-chloro-3,5-diphenyl-1H-pyrazol-1-yl)benzenesulfonamide (B-86);

4-[5-(4-chlorophenyl)-3-(trifluoromethyl)-1H-pyrazol-1-yl]benzenesulfonamide (B-87);

4-[5-phenyl-3-(trifluoromethyl)-1H-pyrazol-1-yl]benzenesulfonamide (B-88);

4-[5-(4-fluorophenyl)-3-(trifluoromethyl)-1H-pyrazol-1-yl]benzenesulfonamide (B-89);

4-[5-(4-methoxyphenyl)-3-(trifluoromethyl)-1H-pyrazol-1-yl]benzenesulfonamide (B-90);

4-[5-(4-chlorophenyl)-3-(difluoromethyl)-1H-pyrazol-1-yl]benzenesulfonamide (B-91);

4-[5-(4-methylphenyl)-3-(trifluoromethyl)-1H-pyrazol-1-yl]benzenesulfonamide (B-92);

4-[4-chloro-5-(4-chlorophenyl)-3-(trifluoromethyl)-1H-pyrazol-1-yl]benzenesulfonamide (B-93);

4-[3-(difluoromethyl)-5-(4-methylphenyl)-1H-pyrazol-1-yl]benzenesulfonamide (B-94);

4-[3-(difluoromethyl)-5-phenyl-1H-pyrazol-1-yl]benzenesulfonamide (B-95);

4-[3-(difluoromethyl)-5-(4-methoxyphenyl)-1H-pyrazol-1-yl]benzenesulfonamide (B-96);

4-[3-cyano-5-(4-fluorophenyl)-1H-pyrazol-1-yl]benzenesulfonamide (B-97);

4-[3-(difluoromethyl)-5-(3-fluoro-4-methoxyphenyl)-1H-pyrazol-1-yl]benzenesulfonamide (B-98);

4-[5-(3-fluoro-4-methoxyphenyl)-3-(trifluoromethyl)-1H-pyrazol-1-yl]benzenesulfonamide (B-99);

4-[4-chloro-5-phenyl-1H-pyrazol-1-yl]benzenesulfonamide (B-100);

4-[5-(4-chlorophenyl)-3-(hydroxymethyl)-1H-pyrazol-1-yl]benzenesulfonamide (B-101);

4-[5-(4-(N,N-dimethylamino)phenyl)-3-(trifluoromethyl)-1H-pyrazol-1-yl]benzenesulfonamide (B-102);

5-(4-fluorophenyl)-6-[4-(methylsulfonyl)phenyl]spiro[2.4]hept-5-ene (B-103);

4-[6-(4-fluorophenyl)spiro[2.4]hept-5-en-5-yl]benzenesulfonamide (B-104);

6-(4-fluorophenyl)-7-[4-(methylsulfonyl)phenyl]spiro[3.4]oct-6-ene (B-105);

5-(3-chloro-4-methoxyphenyl)-6-[4-(methylsulfonyl)phenyl]spiro[2.4]hept-5-ene (B-106);

4-[6-(3-chloro-4-methoxyphenyl)spiro[2.4]hept-5-en-5-yl]benzenesulfonamide (B-107);

5-(3,5-dichloro-4-methoxyphenyl)-6-[4-(methylsulfonyl)phenyl]spiro[2.4]hept-5-ene (B-108);

5-(3-chloro-4-fluorophenyl)-6-[4-(methylsulfonyl)phenyl]spiro[2.4]hept-5-ene (B-109);

4-[6-(3,4-dichlorophenyl)spiro[2.4]hept-5-en-5-yl]benzenesulfonamide (B-110);

2-(3-chloro-4-fluorophenyl)-4-(4-fluorophenyl)-5-(4-methylsulfonylphenyl)thiazole (B-111);

2-(2-chlorophenyl)-4-(4-fluorophenyl)-5-(4-methylsulfonylphenyl)thiazole (B-112);

5-(4-fluorophenyl)-4-(4-methylsulfonylphenyl)-2-methylthiazole (B-113);

4-(4-fluorophenyl)-5-(4-methylsulfonylphenyl)-2-trifluoromethylthiazole (B-114);

4-(4-fluorophenyl)-5-(4-methylsulfonylphenyl)-2-(2-thienyl)thiazole (B-115);

4-(4-fluorophenyl)-5-(4-methylsulfonylphenyl)-2-benzylaminothiazole (B-116);

4-(4-fluorophenyl)-5-(4-methylsulfonylphenyl)-2-(1-propylamino)thiazole (B-117);

2-[(3,5-dichlorophenoxy)methyl)-4-(4-fluorophenyl)-5-[4-(methylsulfonyl)phenyl]thiazole (B-118);

5-(4-fluorophenyl)-4-(4-methylsulfonylphenyl)-2-trifluoromethylthiazole (B-119);

1-methylsulfonyl-4-[1,1-dimethyl-4-(4-fluorophenyl)cyclopenta-2,4-dien-3-yl]benzene (B-120);

4-[4-(4-fluorophenyl)-1,1-dimethylcyclopenta-2,4-dien-3-yl]benzenesulfonamide (B-121);

5-(4-fluorophenyl)-6-[4-(methylsulfonyl)phenyl]spiro[2.4]hepta-4,6-diene (B-122);

4-[6-(4-fluorophenyl)spiro[2.4]hepta-4,6-dien-5-yl]benzenesulfonamide (B-123);

6-(4-fluorophenyl)-2-methoxy-5-[4-(methylsulfonyl)phenyl]-pyridine-3-carbonitrile (B-124);

2-bromo-6-(4-fluorophenyl)-5-[4-(methylsulfonyl)phenyl]-pyridine-3-carbonitrile (B-125);

6-(4-fluorophenyl)-5-[4-(methylsulfonyl)phenyl]-2-phenyl-pyridine-3-carbonitrile (B-126);

4-[2-(4-methylpyridin-2-yl)-4-(trifluoromethyl)-1H-imidazol-1-yl]benzenesulfonamide (B-127);

4-[2-(5-methylpyridin-3-yl)-4-(trifluoromethyl)-1H-imidazol-1-yl]benzenesulfonamide (B-128);

4-[2-(2-methylpyridin-3-yl)-4-(trifluoromethyl)-1H-imidazo-1-yl]benzenesulfonamide (B-129);

3-[1-[4-(methylsulfonyl)phenyl]-4-(trifluoromethyl)-1H-imidazol-2-yl]pyridine (B-130);

2-[1-[4-(methylsulfonyl)phenyl-4-(trifluoromethyl)-1H-imidazol-2-yl]pyridine (B-131);

2-methyl-4-[1-[4-(methylsulfonyl)phenyl-4-(trifluoromethyl)-1H-imidazol-2-yl]pyridine (B-132);

2-methyl-6-[1-[4-(methylsulfonyl)phenyl-4-(trifluoromethyl)-1H-imidazol-2-yl]pyridine (B-133);

4-[2-(6-methylpyridin-3-yl)-4-(trifluoromethyl)-1H-imidazol-1-yl]benzenesulfonamide (B-134);

2-(3,4-difluorophenyl)-1-[4-(methylsulfonyl)phenyl]-4-(trifluoromethyl)-1H-imidazole (B-135);

4-[2-(4-methylphenyl)-4-(trifluoromethyl)-1H-imidazol-1-yl]benzenesulfonamide (B-136);

2-(4-chlorophenyl)-1-[4-(methylsulfonyl)phenyl]-4-methyl-1H-imidazole (B-137);

2-(4-chlorophenyl)-1-[4-(methylsulfonyl)phenyl]-4-phenyl-1H-imidazole (B-138);

2-(4-chlorophenyl)-4-(4-fluorophenyl)-1-[4-(methylsulfonyl)phenyl]-1H-imidazole (B-139);

2-(3-fluoro-4-methoxyphenyl)-1-[4-(methylsulfonyl)phenyl-4-(trifluoromethyl)-1H-imidazole (B-140);

1-[4-(methylsulfonyl)phenyl]-2-phenyl-4-trifluoromethyl-1H-imidazole (B-141);

2-(4-methylphenyl)-1-[4-(methylsulfonyl)phenyl]-4-trifluoromethyl-1H-imidazole (B-142);

4-[2-(3-chloro-4-methylphenyl)-4-(trifluoromethyl)-1H-imidazol-1-yl]benzenesulfonamide (B-143);

2-(3-fluoro-5-methylphenyl)-1-[4-(methylsulfonyl)phenyl]-4-(trifluoromethyl)-1H-imidazole (B-144);

4-[2-(3-fluoro-5-methylphenyl)-4-(trifluoromethyl)-1H-imidazol-1-yl]benzenesulfonamide (B-145);

2-(3-methylphenyl)-1-[4-(methylsulfonyl)phenyl]-4-trifluoromethyl-1H-imidazole (B-146);

4-[2-(3-methylphenyl)-4-trifluoromethyl-1H-imidazol-1-yl]benzenesulfonamide (B-147);

1-[4-(methylsulfonyl)phenyl]-2-(3-chlorophenyl)-4-trifluoromethyl-1H-imidazole (B-148);

4-[2-(3-chlorophenyl)-4-trifluoromethyl-1H-imidazol-1-yl]benzenesulfonamide (B-149);

4-[2-phenyl-4-trifluoromethyl-1H-imidazol-1-yl]benzenesulfonamide (B-150);

4-[2-(4-methoxy-3-chlorophenyl)-4-trifluoromethyl-1H-imidazol-1-yl]benzenesulfonamide (B-151);

1-allyl-4-(4-fluorophenyl)-3-[4-(methylsulfonyl)phenyl]-5-(trifluoromethyl)-1H-pyrazole (B-152);

4-[1-ethyl-4-(4-fluorophenyl)-5-(trifluoromethyl)-1H-pyrazol-3-yl]benzenesulfonamide (B-153);

N-phenyl-[4-(4-fluorophenyl)-3-[4-(methylsulfonyl)phenyl]-5-(trifluoromethyl)-1H-pyrazol-1-yl]acetamide (B-154);

ethyl [4-(4-fluorophenyl)-3-[4-(methylsulfonyl)phenyl]-5-(trifluoromethyl)-1H-pyrazol-1-yl]acetate (B-155);

4-(4-fluorophenyl)-3-[4-(methylsulfonyl)phenyl]-1-(2-phenylethyl)-1H-pyrazole (B-156);

4-(4-fluorophenyl)-3-[4-(methylsulfonyl)phenyl]-1-(2-phenylethyl)-5-(trifluoromethyl)pyrazole (B-157);

1-ethyl-4-(4-fluorophenyl)-3-[4-(methylsulfonyl)phenyl]-5-(trifluoromethyl)-1H-pyrazole (B-158);

5-(4-fluorophenyl)-4-(4-methylsulfonylphenyl)-2-trifluoromethyl-1H-imidazole (B-159);

4-[4-(methylsulfonyl)phenyl]-5-(2-thiophenyl)-2-(trifluoromethyl)-1H-imidazole (B-160);

5-(4-fluorophenyl)-2-methoxy-4-[4-(methylsulfonyl)phenyl]-6-(trifluoromethyl)pyridine (B-161);

2-ethoxy-5-(4-fluorophenyl)-4-[4-(methylsulfonyl)phenyl]-6-(trifluoromethyl)pyridine (B-162);

5-(4-fluorophenyl)-4-[4-(methylsulfonyl)phenyl]-2-(2-propynyloxy)-6-(trifluoromethyl)pyridine (B-163);

2-bromo-5-(4-fluorophenyl)-4-[4-(methylsulfonyl)phenyl]-6-(trifluoromethyl)pyridine (B-164);

4-[2-(3-chloro-4-methoxyphenyl)-4,5-difluorophenyl]benzenesulfonamide (B-165);

1-(4-fluorophenyl)-2-[4-(methylsulfonyl)phenyl]benzene (B-166);

5-difluoromethyl-4-(4-methylsulfonylphenyl)-3-phenylisoxazole (B-167);

4-[3-ethyl-5-phenylisoxazol-4-yl]benzenesulfonamide (B-168);

4-[5-difluoromethyl-3-phenylisoxazol-4-yl]benzenesulfonamide (B-169);

4-[5-hydroxymethyl-3-phenylisoxazol-4-yl]benzenesulfonamide (B-170);

4-[5-methyl-3-phenyl-isoxazol-4-yl]benzenesulfonamide (B-171);

1-[2-(4-fluorophenyl)cyclopenten-1-yl]-4-(methylsulfonyl)benzene (B-172);

1-[2-(4-fluoro-2-methylphenyl)cyclopenten-1-yl]-4-(methylsulfonyl)benzene (B-173);

1-[2-(4-chlorophenyl)cyclopenten-1-yl]-4-(methylsulfonyl)benzene (B-174);

1-[2-(2,4-dichlorophenyl)cyclopenten-1-yl]-4-(methylsulfonyl)benzene (B-175);

1-[2-(4-trifluoromethylphenyl)cyclopenten-1-yl]-4-(methylsulfonyl)benzene (B-176);

1-[2-(4-methylthiophenyl)cyclopenten-1-yl]-4-(methylsulfonyl)benzene (B-177);

1-[2-(4-fluorophenyl)-4,4-dimethylcyclopenten-1-yl]-4-(methylsulfonyl)benzene (B-178);

4-[2-(4-fluorophenyl)-4,4-dimethylcyclopenten-1-yl]benzenesulfonamide (B-179);

1-[2-(4-chlorophenyl)-4,4-dimethylcyclopenten-1-yl]-4-(methylsulfonyl)benzene (B-180);

4-[2-(4-chlorophenyl)-4,4-dimethylcyclopenten-1-yl]benzenesulfonamide (B-181);

4-[2-(4-fluorophenyl)cyclopenten-1-yl]benzenesulfonamide (B-182);

4-[2-(4-chlorophenyl)cyclopenten-1-yl]benzenesulfonamide (B-183);

1-[2-(4-methoxyphenyl)cyclopenten-1-yl]-4-(methylsulfonyl)benzene (B-184);

1-[2-(2,3-difluorophenyl)cyclopenten-1-yl]-4-(methylsulfonyl)benzene (B-185);

4-[2-(3-fluoro-4-methoxyphenyl)cyclopenten-1-yl]benzenesulfonamide (B-186);

1-[2-(3-chloro-4-methoxyphenyl)cyclopenten-1-yl]-4-(methylsulfonyl)benzene (B-187);

4-[2-(3-chloro-4-fluorophenyl)cyclopenten-1-yl]benzenesulfonamide (B-188);

4-[2-(2-methylpyridin-5-yl)cyclopenten-1-yl]benzenesulfonamide (B-189);

ethyl 2-[4-(4-fluorophenyl)-5-[4-(methylsulfonyl)phenyl]oxazol-2-yl]-2-benzyl-acetate (B-190);

2-[4-(4-fluorophenyl)-5-[4-(methylsulfonyl)phenyl]oxazol-2-yl]acetic acid (B-191);

2-(tert-butyl)-4-(4-fluorophenyl)-5-[4-(methylsulfonyl)phenyl]oxazole (B-192);

4-(4-fluorophenyl)-5-[4-(methylsulfonyl)phenyl]-2-phenyloxazole (B-193);

4-(4-fluorophenyl)-2-methyl-5-[4-(methylsulfonyl)phenyl]oxazole (B-194);

4-[5-(3-fluoro-4-methoxyphenyl)-2-trifluoromethyl-4-oxazolyl]benzenesulfonamide (B-195);

6-chloro-7-(1,1-dimethylethyl)-2-trifluoromethyl-2H-1-benzopyran-3-carboxylic acid (B-196);

6-chloro-8-methyl-2-trifluoromethyl-2H-1-benzopyran-3-carboxylic acid (B-197);

5,5-dimethyl-3-(3-fluorophenyl)-4-methylsulfonyl-2(5H)-furanone (B-198);

6-chloro-2-trifluoromethyl-2H-1-benzothiopyran-3-carboxylic acid (B-199);

4-[5-(4-chlorophenyl)-3-(trifluoromethyl)-1H-pyrazol-1-yl]benzenesulfonamide (B-200);

4-[5-(4-methylphenyl)-3-(trifluoromethyl)-1H-pyrazol-1-yl]benzenesulfonamide (B-201);

4-[5-(3-fluoro-4-methoxyphenyl)-3-(difluoromethyl)-1H-pyrazol-1-yl]benzenesulfonamide (B-202);

3-[1-[4-(methylsulfonyl)phenyl]-4-trifluoromethyl-1H-imidazol-2-yl]pyridine (B-203);

2-methyl-5-[1-[4-(methylsulfonyl)phenyl]-4-trifluoromethyl-1H-imidazol-2-yl]pyridine (B-204);

4-[2-(5-methylpyridin-3-yl)-4-(trifluoromethyl)-1H-imidazol-1-yl]benzenesulfonamide (B-205);

4-[5-methyl-3-phenylisoxazol-4-yl]benzenesulfonamide (B-206);

4-[5-hydroxymethyl-3-phenylisoxazol-4-yl]benzenesulfonamide (B-207);

[2-trifluoromethyl-5-(3,4-difluorophenyl)-4-oxazolyl]benzenesulfonamide (B-208);

4-[2-methyl-4-phenyl-5-oxazolyl]benzenesulfonamide (B-209);

4-[5-(2-fluoro-4-methoxyphenyl)-2-trifluoromethyl-4-oxazolyl]benzenesulfonamide (B-210);

[2-(2-chloro-6-fluoro-phenylamino)-5-methyl-phenyl]-acetic acid or COX 189 (lumiracoxib; B-211);

N-(4-Nitro-2-phenoxy-phenyl)-methanesulfonamide or nimesulide (B-212);

N-[6-(2,4-difluoro-phenoxy)-1-oxo-indan-5-yl]-methanesulfonamide or flosulide (B-213);

N-[6-(2,4-Difluoro-phenylsulfanyl)-1-oxo-1H-inden-5-yl]-methanesulfonamide, soldium salt or L-745337 (B-214);

N-[5-(4-fluoro-phenylsulfanyl)-thiophen-2-yl]-methanesulfonamide or RWJ-63556 (B-215);

3-(3,4-Difluoro-phenoxy)-4-(4-methanesulfonyl-phenyl)-5-methyl-5-(2,2,2-trifluoro-ethyl)-5H-furan-2-one or L-784512 or L-784512 (B-216);

(5Z)-2-amino-5-[[3,5-bis(1,1-dimethylethyl)-4-hydroxyphenyl]methylene]-4(5H)-thiazolone or darbufelone (B-217);

CS-502 (B-218);

LAS-34475 (B-219);

LAS-34555 (B-220);

S-33516 (B-221);

SD-8381 (B-222);

L-783003 (B-223);

N-[3-(formylamino)-4-oxo-6-phenoxy-4H-1-benzopyran-7-yl]-methanesulfonamide or T-614 (B-224);

D-1367 (B-225);

L-748731 (B-226);

(6aR,10aR)-3-(1,1-dimethylheptyl)-6a,7,10,10a-tetrahydro-1-hydroxy-6,6-dimethyl-6H-dibenzo[b,d]pyran-9-carboxylic acid or CT3 (B-227);

CGP-28238 (B-228);

4-[[3,5-bis(1,1-dimethylethyl)-4-hydroxyphenyl]methylene]dihydro-2-methyl-2H-1,2-oxazin-3(4H)-one or BF-389 (B-229);

GR-253035 (B-230);

6-dioxo-9H-purin-8-yl-cinnamic acid (B-231);

S-2474 (B-232);

4-[4-(methyl)-sulfonyl)phenyl]-3-phenyl-2(5H)-furanone;

4-(5-methyl-3-phenyl-4-isoxazolyl);

2-(6-methylpyrid-3-yl)-3-(4-methylsulfonylphenyl)-5-chloropyridine;

4-[5-(4-methylphenyl)-3-(trifluoromethyl)-1H-pyrazol-1-yl];

N-[[4-(5-methyl-3-phenyl-4-isoxazolyl)phenyl]sulfonyl];

4-[5-(3-fluoro-4-methoxyphenyl)-3-difluoromethyl)-1H-pyrazol-1-yl]benzenesulfonamide;

(S)-6,8-dichloro-2-(trifluoromethyl)-2H-1-benzopyran-3-carboxylic acid;

2-(3,4-difluorophenyl)-4-(3-hydroxy-3-methylbutoxy)-5-[4-(methylsulfonyl)phenyl]-3(2H)-pyridzainone;

2-trifluoromethyl-3H-naptho[2,1-b]pyran-3-carboxylic acid;

6-chloro-7-(1,1-dimethylethyl)-2-trifluoromethyl-2H-1-benzopyran-3-carboxylic acid;

[2-(2,4-dichloro-6-ethyl-3,5-dimethyl-phenylamino)-5-propyl-phenyl]-acetic acid.

TABLE 3x
Examples of Cyclooxygenase-2 Selective Inhibitors as Embodiments

| Compound | |
|---|---|
| Number | Structural Formula |
| B-26 | 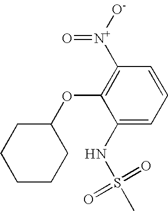 |
| | N-(2-cyclohexyloxynitrophenyl) methane |
| | sulfonamide or NS-398; |
| B-27 | 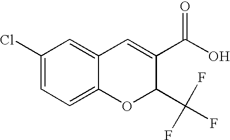 |
| | 6-chloro-2-trifluoromethyl-2H-1-benzopyran- |
| | 3-carboxylic acid; |
| B-28 | 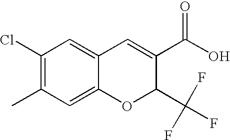 |
| | 6-chloro-7-methyl-2-trifluoromethyl-2H-1- |
| | benzopyran-3-carboxylic acid |
| B-29 | 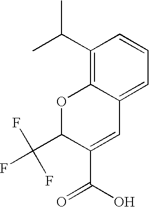 |
| | 8-(1-methylethyl)-2-trifluoromethyl-2H-1- |
| | benzopyran-3-carboxylic acid |
| B-30 | 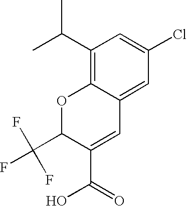 |
| | 6-chloro-8-(1-methylethyl)-2-trifluoromethyl- |
| | 2H-1-benzopyran-3-carboxylic acid; |
| B-31 | 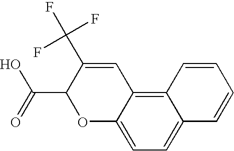 |
| | 2-trifluoromethyl-3H-naphtho[2,1-b]pyran-3- |
| | carboxylic acid; |
| B-32 | 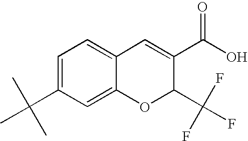 |
| | 7-(1,1-dimethylethyl)-2-trifluoromethyl-2H- |
| | 1-benzopyran-3-carboxylic |
| B-33 | 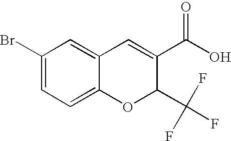 |
| | 6-bromo-2-trifluoromethyl-2H-1-benzopyran- |
| | 3-carboxylic acid; |
| B-34 | 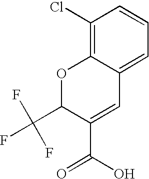 |
| | 8-chloro-2-trifluoromethyl-2H-1- |
| | benzopyran-3-carboxylic acid; |
| B-35 | 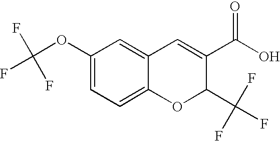 |
| | 6-trifluoromethoxy-2-trifluoromethyl- |
| | 2H-1-benzopyran-3-carboxylic |
| B-36 | 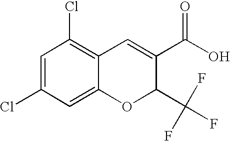 |
| | 5,7-dichloro-2-trifluoromethyl-2H-1- |
| | benzopyran-3-carboxylic acid; |
| B-37 | 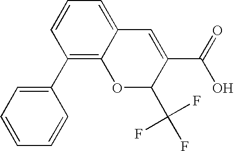 |
| | 8-phenyl-2-trifluoromethyl-2H-1- |
| | benzopyran-3-carboxylic acid; |
| B-38 | 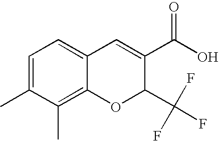 |
| | 7,8-dimethyl-2-trifluoromethyl-2H-1- |
| | benzopyran-3-carboxylic acid; |
| B-39 | 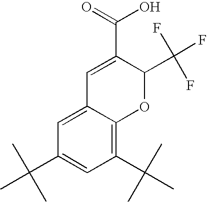 |
| | 6,8-bis(dimethylethyl)-2-trifluoromethyl- |
| | 2H-1-benzopyran-3-carboxylic acid; |
| B-40 | 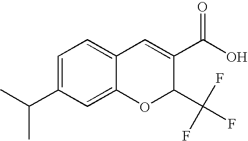 |
| | 7-(1-methylethyl)-2-trifluoromethyl-2H-1- |
| | benzopyran-3-carboxylic acid |
| B-41 | 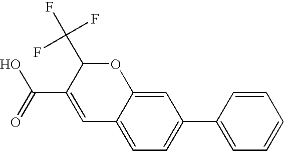 |
| | 7-phenyl-2-trifluoromethyl-2H-1-benzopyran- |
| | 3-carboxylic acid |
| B-42 | 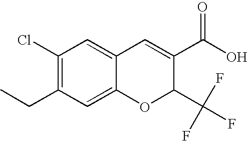 |
| | 6-chloro-7-ethyl-2-trifluoromethyl-2H-1- |
| | benzopyran-3-carboxylic acid |
| B-43 | 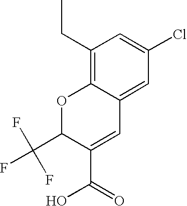 |
| | 6-chloro-8-ethyl-2-trifluoromethyl-2H-1- |
| | benzopyran-3-carboxylic acid |
| B-44 | 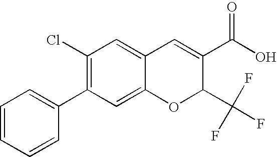 |
| | 6-chloro-7-phenyl-2-trifluoromethyl-2H-1- |
| | benzopyran-3-carboxylic acid |
| B-45 | 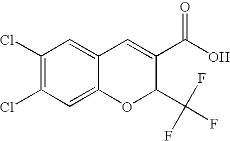 |
| | 6,7-dichloro-2-trifluoromethyl-2H-1- |
| | benzopyran-3-carboxylic acid |
| B-46 | 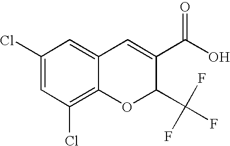 |
| | 6,8-dichloro-2-trifluoromethyl-2H-1- |
| | benzopyran-3-carboxylic acid |
| B-47 | 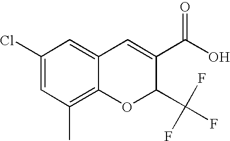 |
| | 6-chloro-8-methyl-2-trifluoromethyl-2H-1- |
| | benzopyran-3-carboxylic acid |
| B-48 | 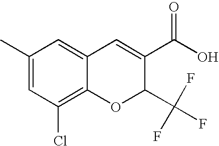 |
| | 8-chloro-6-methyl-2-trifluoromethyl-2H-1- |
| | benzopyran-3-carboxylic acid |
| B-49 | 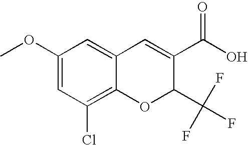 |
| | 8-chloro-6-methoxy-2-trifluoromethyl-2H-1- |
| | benzopyran-3-carboxylic acid |
| B-50 | 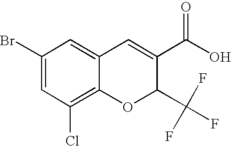 |
| | 6-bromo-8-chloro-2-trifluoromethyl-2H-1- |
| | benzopyran-3-carboxylic acid |
| B-51 | 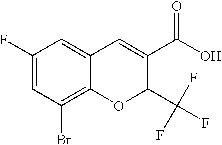 |
| | 8-bromo-6-fluoro-2-trifluoromethyl-2H-1- |
| | benzopyran-3-carboxylic acid |
| B-52 | 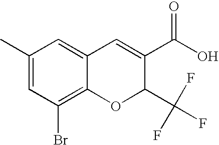 |
| | 8-bromo-6-methyl-2-trifluoromethyl-2H-1- |
| | benzopyran-3-carboxylic acid |
| B-53 | 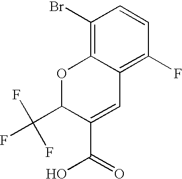 |
| | 8-bromo-5-fluoro-2-trifluoromethyl-2H-1- |
| | benzopyran-3-carboxylic acid; |
| B-54 | 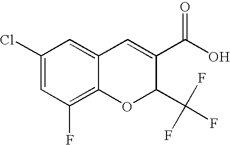 |
| | 6-chloro-8-fluoro-2-trifluoromethyl-2H-1- |
| | benzopyran-3-carboxylic acid |
| B-55 | 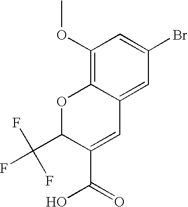 |
| | 6-bromo-8-methoxy-2-trifluoromethyl-2H-1- |
| | benzopyran-3-carboxylic acid |
| B-56 | 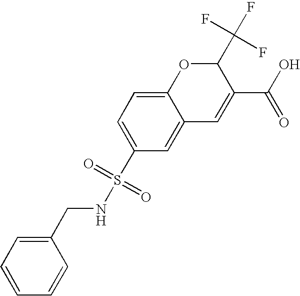 |
| | 6-[[(phenylmethyl)amino]sulfonyl]-2- |
| | trifluoromethyl-2H-1-benzopyran-3-carboxylic |
| B-57 | 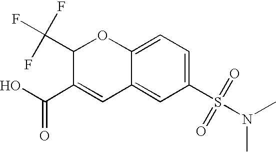 |
| | 6-[(dimethylamino)sulfonyl]-2-trifluoromethyl- |
| | 2H-1-benzopyran-3-carbox |
| B-61 | 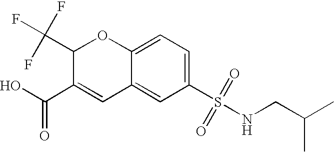 |
| | 6-[(2-methylpropyl)aminosulfonyl]-2-trifluoromethyl- |
| | 2H-1-benzopyran-3-carboxylic a |
| B-62 | 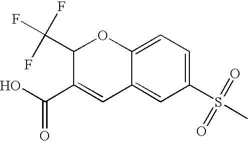 |
| | 6-methylsulfonyl-2-trifluoromethyl-2H-1-benzopyran- |
| | 3-carboxylic acid |
| B-63 | 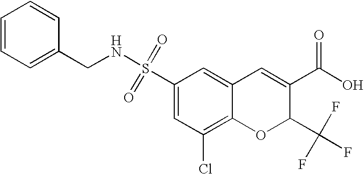 |
| | 8-chloro-6-[[(phenylmethyl)amino]sulfonyl]-2- |
| | trifluoromethyl-2H-1-benzopyran-3-carboxylic acid; |
| B-64 | 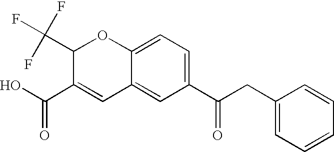 |
| | 6-phenylacetyl-2-trifluoromethyl-2H-1-benzopyran- |
| | 3-carboxylic acid; |
| B-65 | 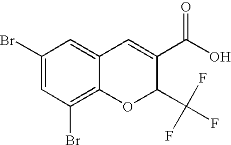 |
| | 6,8-dibromo-2-trifluoromethyl-2H-1-benzopyran- |
| | 3-carboxylic acid |
| B-66 | 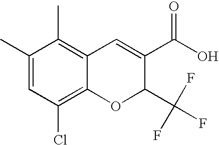 |
| | 8-chloro-5,6-dimethyl-2-trifluoromethyl-2H-1- |
| | benzopyran-3-carboxylic acid |
| B-67 | 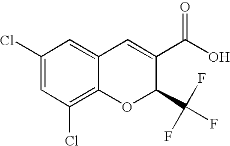 |
| | 6,8-dichloro-(S)-2-trifluoromethyl-2H-1- |
| | benzopyran-3-carboxylic acid |
| B-68 | 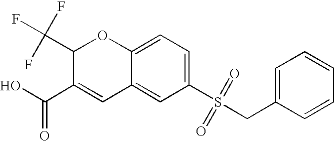 |
| | 6-benzylsulfonyl-2-trifluoromethyl-2H-1- |
| | benzopyran-3-carboxylic acid |
| B-69 | 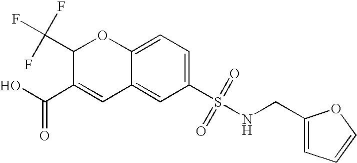 |
| | 6-[[N-(2-furylmethyl)amino]sulfonyl]-2- |
| | trifluoromethyl-2H-1-benzopyran-3-carboxylic acid; |
| B-70 | 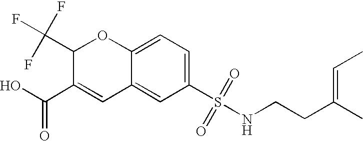 |
| | 6-[[N-(2-phenylethyl)amino]sulfonyl]-2- |
| | trifluoromethyl-2H-1-benzopy-3-carboxylic acid; |
| B-71 | 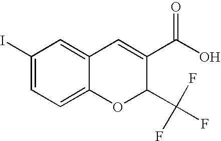 |
| | 6-iodo-2-trifluoromethyl-2H-1-benzopyran-3- |
| | carboxylic acid; |
| B-72 | 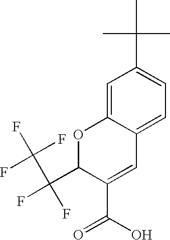 |
| | 7-(1,1-dimethylethyl)-2-pentafluoroethyl-2H- |
| | 1-benzopyran-3-carboxylic acid; |
| B-73 | 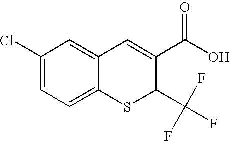 |
| | 6-chloro-2-trifluoromethyl-2H-1-benzothiopyran- |
| | 3-carboxylic acid |
| B-74 | 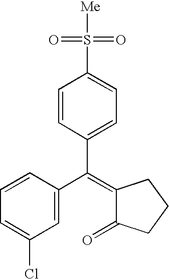 |
| | 3-[(3-chloro-phenyl)-(4-methanesulfonyl-phenyl)- |
| | methylene]-dihydro-furan-2-one or BMS-347070; |
| B-75 | 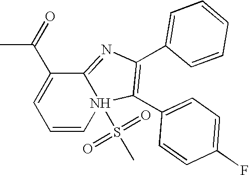 |
| | 8-acetyl-3-(4-fluorophenyl)-2-(4- |
| | methylsulfonyl)pbenyl-imidazo(1,2-a)pyridine |
| B-76 | 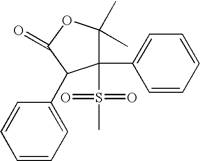 |
| | 5,5-dimethyl-4-(4-methylsulfonyl)phenyl-3- |
| | phenyl-2-(5H)-furanone; |
| B-77 | 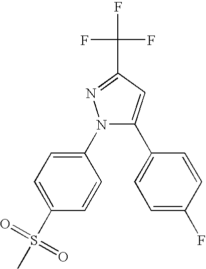 |
| | 5-(4-fluorophenyl)-1-[4-(methylsulfonyl) |
| | phenyl]-3-(trifluoromethyl)pyrazole |
| B-78 | 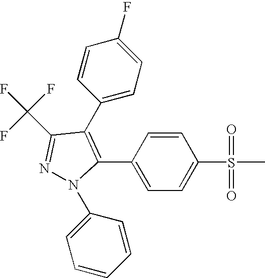 |
| | 4-(4-fluorophenyl)-5-[4-(methylsulfonyl) |
| | phenyl]-1-phenyl-3-(trifluoromethyl)pyrazole; |
| B-79 | 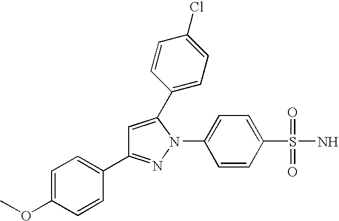 |
| | 4-(5-(4-chlorophenyl)-3-(4-methoxyphenyl)-1H- |
| | pyrazol-1-yl)benzenesulfonamide; |
| B-80 | 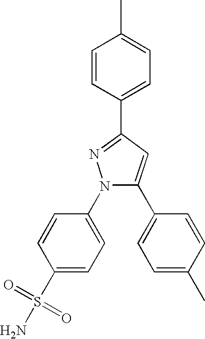 |
| | 4-(3,5-bis(4-methylphenyl)-1H-pyrazol-1- |
| | yl)benzenesulfonamide; |
| B-81 | 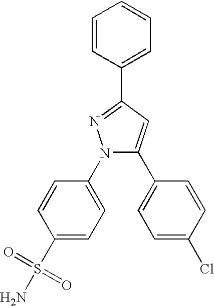 |
| | 4-(5-(4-chlorophenyl)-3-phenyl-1H-pyrazol-1- |
| | yl)benzenesulfonamide; |
| B-82 | 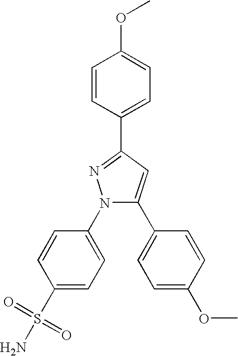 |
| | 4-(3,5-bis(4-methoxyphenyl)-1H-pyrazol-1-yl) |
| | benzenesulfonamide; |
| B-83 | 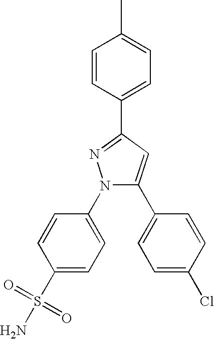 |
| | 4-(5-(4-chlorophenyl)-3-(4-methylphenyl)-1H- |
| | pyrazol-1-yl)benzenesulfonamide |
| B-84 | 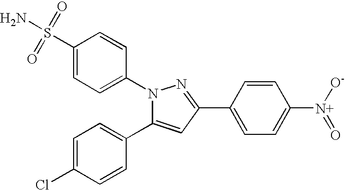 |
| | 4-(5-(4-chlorophenyl)-3-(4-nitrophenyl)-1H- |
| | pyrazol-1-yl)benzenesulfonamide; |
| B-85 | 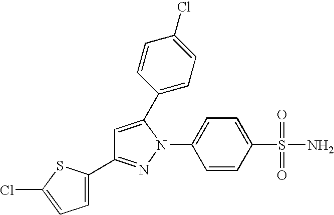 |
| | 4-(5-(4-chlorophenyl)-3-(5-chloro-2-thienyl)- |
| | 1H-pyrazol-1-yl)benzenesulfonamide |
| B-86 | 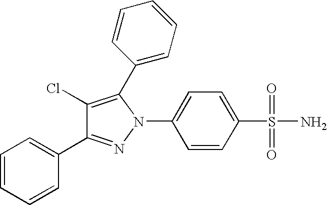 |
| | 4-(4-chloro-3,5-diphenyl-1H-pyrazol-1- |
| | yl)benzenesulfonamide; |
| B-87 | 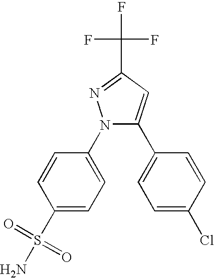 |
| | 4-[5-(4-chlorophenyl)-3-(trifluoromethyl)-1H- |
| | pyrazol-1-yl]benzenesulfonamide |
| B-88 | 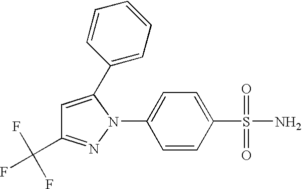 |
| | 4-[5-phenyl-3-(trifluoromethyl)-1H-pyrazol-1- |
| | yl]benzenesulfonamide; |
| B-89 | 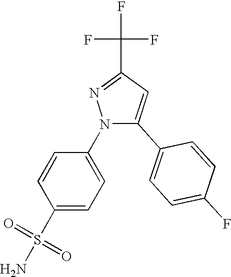 |
| | 4-[5-(4-fluorophenyl)-3-(trifluoromethyl)-1H- |
| | pyrazol-1-yl]benzenesulfonamide; |
| B-90 | 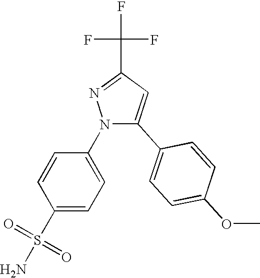 |
| | 4[5-(4-methoxyphenyl)-3-(trifluoromethyl)- |
| | 1H-pyrazol-1-yl]benzenesulfonamide; |
| B-91 | 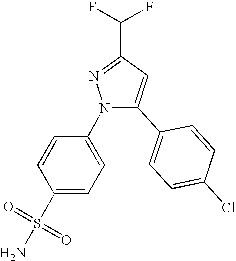 |
| | 4-[5-(4-chlorophenyl)-3-(difluoromethyl)- |
| | 1H-pyrazol-1-yl]benzenesulfonamide; |
| B-92 | 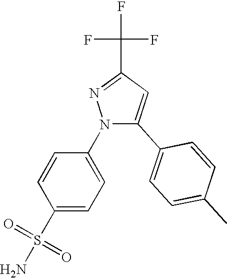 |
| | 4-[5-(4-methylphenyl)-3-(trifluoromethyl)- |
| | 1H-pyrazol-1-yl]benzenesulfonamide; |
| B-93 | 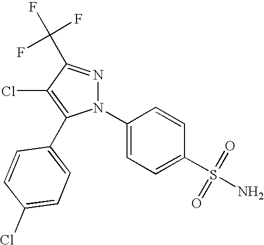 |
| | 4-[4-chloro-5-(4-chlorophenyl)-3-(trifluoromethyl)- |
| | 1H-pyrazol-1-yl]benzenesulfonamide; |
| B-94 | 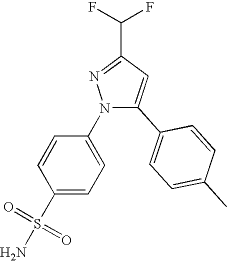 |
| | 4-[3-(difluoromethyl)-5-(4-methylphenyl)- |
| | 1H-pyrazol-1-yl]benzenesulfonamide |
| B-95 | 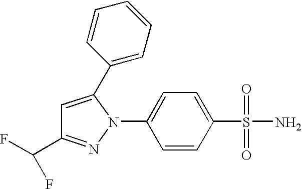 |
| | 4-[3-(difluoromethyl)-5-phenyl-1H- |
| | pyrazol-1-yl]benzenesulfonamide; |
| B-96 | 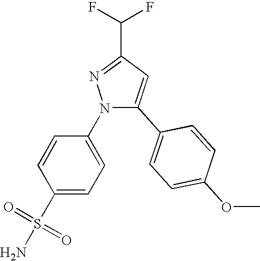 |
| | 4-[3-(difluoromethyl)-5-(4-methoxyphenyl)-1H- |
| | pyrazol-1-yl]benzenesulfonamide; |
| B-97 | 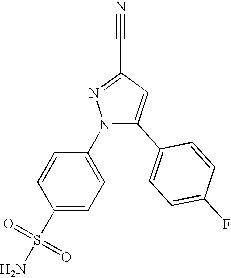 |
| | 4-[3-cyano-5-(4-fluorophenyl)-1H-pyrazol-1- |
| | yl]benzenesulfonamide; |
| B-98 | 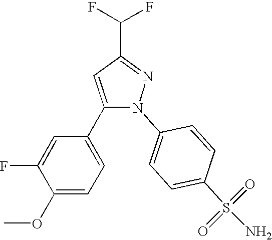 |
| | 4-[3-(difluoromethyl)-5-(3-fluoro-4- |
| | methoxyphenyl)-1H-pyrazol-1-yl] |
| | benzenesulfonamide; |
| B-99 | 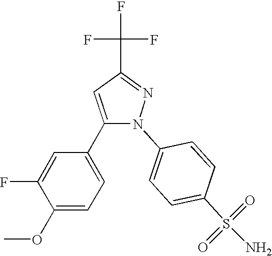 |
| | 4-[5-(3-fluoro-4-methoxyphenyl)-3- |
| | (trifluoromethyl)-1H-pyrazol-1-yl] |
| | benzenesulfonamide; |
| B-100 | 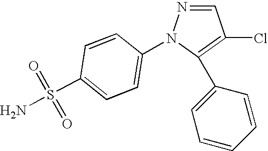 |
| | 4-[4-chloro-5-phenyl-1H-pyrazol-1- |
| | yl]benzenesulfonamide; |
| B-101 | 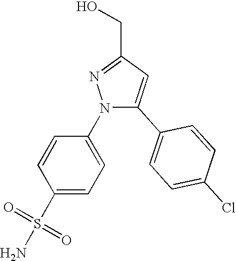 |
| | 4-[5-(4-chlorophenyl)-3-(hydroxymethyl)- |
| | 1H-pyrazol-1-yl]benzenesulfonamide; |
| B-102 | 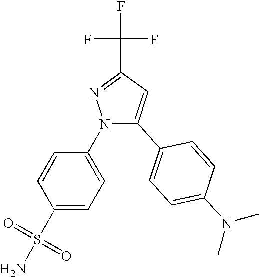 |
| | 4-[5-(4-(N,N-dimethylamino)phenyl)-3- |
| | (trifluoromethyl)-1H-pyrazol-1- |
| | yl]benzenesulfonamide; |
| B-103 | 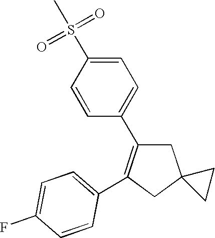 |
| | 5-(4-fluorophenyl)-6-[4- |
| | (methylsulfonyl)phenyl]spiro[2.4]hept-5-ene; |
| B-104 | 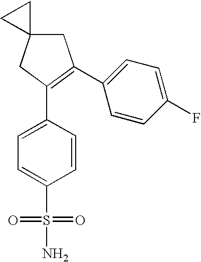 |
| | 4-[6-(4-fluorophenyl)spiro[2.4]hept-5- |
| | en-5-yl]benzenesulfonamide; |
| B-105 | 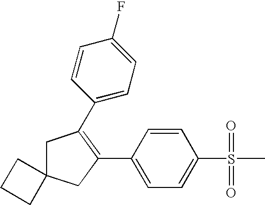 |
| | 6-(4-fluorophenyl)-7-[4-methylsulfonyl) |
| | phenyl]spiro[3.4]oct-6-ene; |
| B-106 | 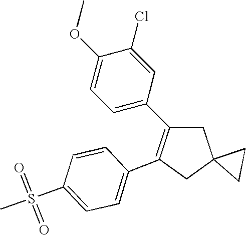 |
| | 5-(3-chloro-4-methoxypbenyl)-6-[4- |
| | (methylsulfonyl)phenyl]spira[2.4]hept-5-ene; |
| B-107 | 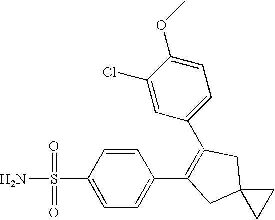 |
| | 4-[6-(3-ch1oro-4-methoxyphenyl)spiro[2.4] |
| | hept-5-en-5-yl]benzenesulfonamide; |
| B-108 | 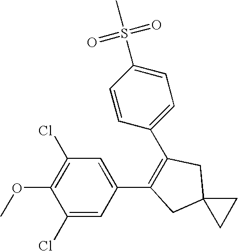 |
| | 5-(3,5-dichloro-4-methoxyphenyl)-6-[4- |
| | (methylsulfonyl)phenyl]spiro[2.4]hept- |
| | 5-ene; |
| B-109 | 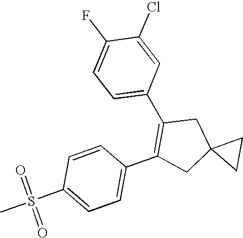 |
| | 5-(3-chloro-4-fluorophenyl)-6-[4- |
| | (methylsulfonyl)phenyl]spiro[2.4]hept- |
| | 5-ene; |
| B-110 | 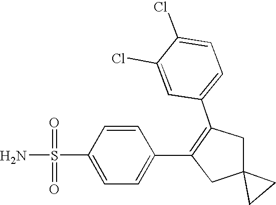 |
| | 4-[6-(3,4-dichlorophenyl)spiro[2.4] |
| | hept-5-en-5-yl]benzenesulfonamide; |
| B-111 | 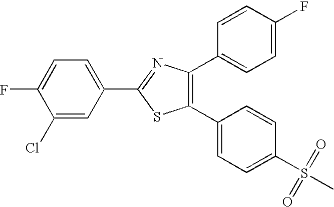 |
| | 2-(3-chloro-4-fluorophenyl)-4-(4- |
| | fluorophenyl)-5-(4- |
| | methylsulfonylphenyl)thiazole; |
| B-112 | 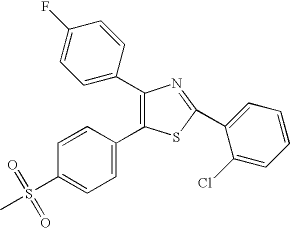 |
| | 2-(2-chlorophenyl)-4-(4-fluorophenyl)-5- |
| | (4-methylsulfonylphenyl)thiazole; |
| B-113 | 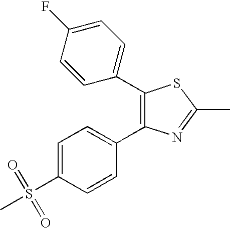 |
| | 5-(4-fluorophenyl)-4-(4- |
| | methylsulfonylphenyl)-2-methylthiazole; |
| B-114 | 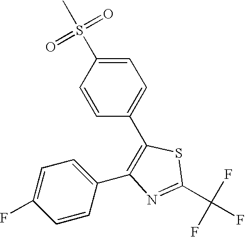 |
| | 4-(4-fluorophenyl)-5-(4- |
| | methylsulfonylphenyl)-2-trifluoromethylthiazole; |
| B-115 | 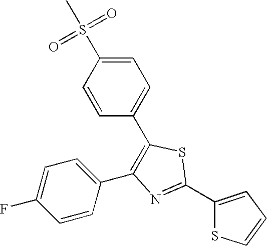 |
| | 4-(4-fluorophenyl)-5-(4-methylsulfonylphenyl)- |
| | 2-(2-thienyl)thiazole; |
| B-116 | 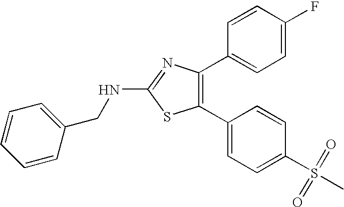 |
| | 4-(4-fluorophenyl)-5-(4-methylsulfonylphenyl)- |
| | 2-benzylaminothiazole; |
| B-117 | 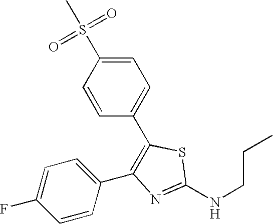 |
| | 4-(4-fluorophenyl)-5-(4-methylsulfonylphenyl)- |
| | 2-(1-propylamino)thiazole; |
| B-118 | 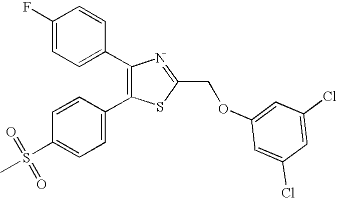 |
| | 2-((3,5-dichlorophenoxy)methyl)-4-(4- |
| | fluorophenyl)-5-[4-(methylsulfonyl) |
| | phenyl]thiazole; |
| B-119 | 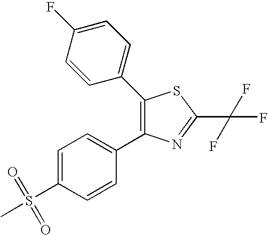 |
| | 5-(4-fluorophenyl)-4-(4- |
| | methylsulfonylphenyl)-2-trifluoromethylthiazole; |
| B-120 | 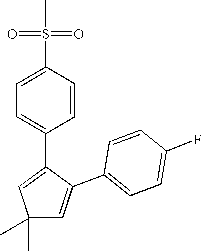 |
| | 1-methylsulfonyl-4-[1,1-dimethyl-4-(4- |
| | fluorophenyl)cyclopenta-2,4-dien-3-yl]benzene; |
| B-121 | 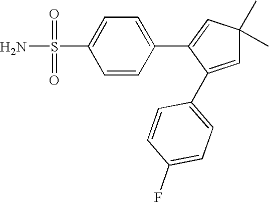 |
| | 4-[4-(4-fluorophenyl)-1,1- |
| | dimethylcyclopenta-2,4-dien-3-yl] |
| | benzenesulfonamide; |
| B-122 | 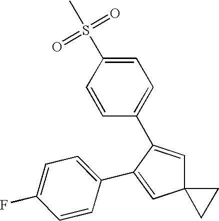 |
| | 5-(4-fluorophenyl)-6-[4- |
| | (methylsulfonyl)phenyl]spiro[2.4] |
| | hepta-4,6-diene; |
| B-123 | 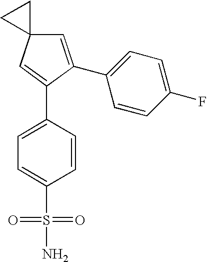 |
| | 4-[6-(4-fluorophenyl)spiro[2.4]hepta- |
| | 4,6-dien-5-yl]benzenesulfonamide; |
| B-124 | 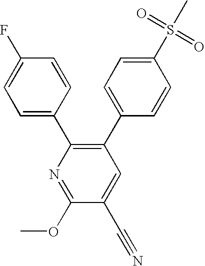 |
| | 6-(4-fluorophenyl)-2-methoxy-5-[4- |
| | (methylsulfonyl)phenyl]- |
| | pyridine-3-carbonitrile; |
| B-125 | 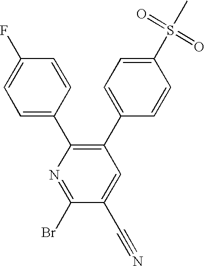 |
| | 2-bromo-6-(4-fluorophenyl)-5-[4- |
| | (methylsulfonyl)phenyl]- |
| | pyridine-3-carbonitrile; |
| B-126 | 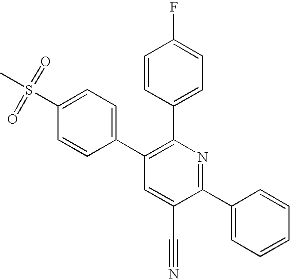 |
| | 6-(4-fluorophenyl)-5-[4- |
| | (methylsulfonyl)phenyl]-2-phenyl- |
| | pyridine-3-carbonitrile; |
| B-127 | 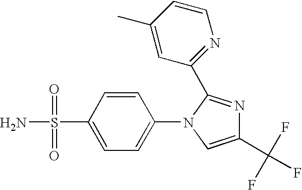 |
| | 4-[2-(4-methylpyridin-2-yl)-4- |
| | (trifluoromethyl)-1H-imidazol-1-yl] |
| | benzenesulfonamide; |
| B-128 | 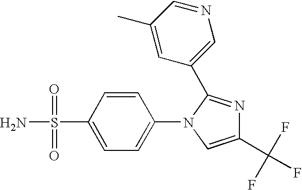 |
| | 4-[2-(5-methylpyridin-3-yl)-4- |
| | (trifluoromethyl)-1H-imidazol-1-yl] |
| | benzenesulfonamide; |
| B-129 | 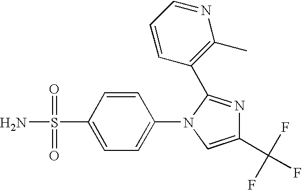 |
| | 4-[2-(2-methylpyridin-3-yl)-4- |
| | (trifluoromethyl)-1H-imidazol-1-yl] |
| | benzenesulfonamide; |
| B-130 | 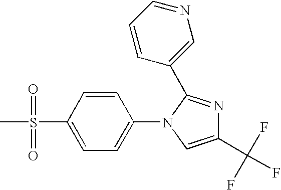 |
| | 3-[1-[4-(methylsulfonyl)phenyl]-4- |
| | (trifluoromethyl)-1H-imidazol-2-yl]pyridine; |
| B-131 | 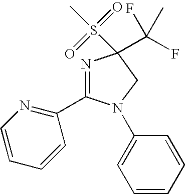 |
| | 2-[1-[4-(methylsulfonyl)phenyl-4- |
| | (trifluoromethyl)]-1H-imidazol-2-yl]pyridine |
| B-132 | 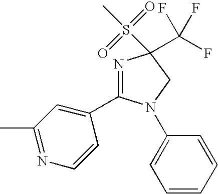 |
| | 2-methyl-4-[1-[4-(methylsulfonyl)phenyl-4- |
| | (trifluoromethyl)]-1H-imidazol-2- |
| | yl]pyridine; |
| B-133 | 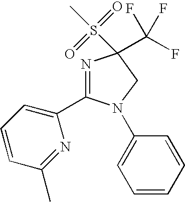 |
| | 2-methyl-6-[1-[4-(methylsulfonyl)phenyl-4- |
| | (trifluoromethyl)]-1H-imidazol-2-yl]pyridine; |
| B-134 | 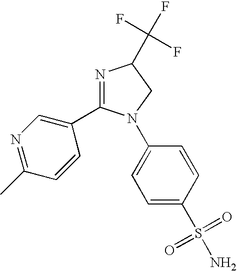 |
| | 4-[2-(6-methylpyridin-3-yl)-4-(trifluoromethyl)- |
| | 1H-imidazol-1-yl]benzenesulfonamide |
| B-135 | 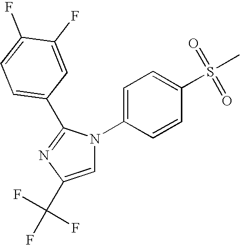 |
| | 2-(3,4-difluorophenyl)-1-[4-(methylsulfonyl) |
| | phenyl]-4-(trifluoromethyl)-1H-imidazole; |
| B-136 | 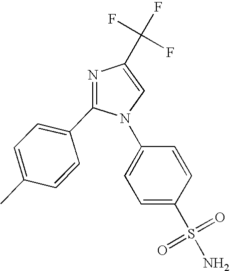 |
| | 4-[2-(4-methylphenyl)-4-(trifluoromethyl)- |
| | 1H-imidazol-1-yl]benzenesulfonamide |
| B-137 | 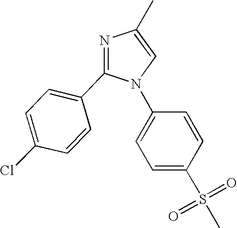 |
| | 2-(4-chlorophenyl)-1-[4-(methylsulfonyl) |
| | phenyl]-4-methyl-1H-imidazole |
| B-138 | 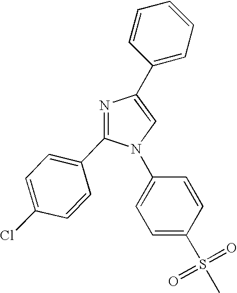 |
| | 2-(4-chlorophenyl)-1-[4-(methylsulfonyl) |
| | phenyl]-4-phenyl-1H-imidazole; |
| B-139 | 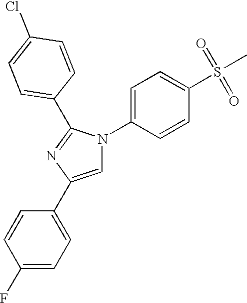 |
| | 2-(4-chlorophenyl)-4-(4-fluorophenyl)-1-[4- |
| | (methylsulfonyl)phenyl]-1H-imidazole; |
| B-140 | 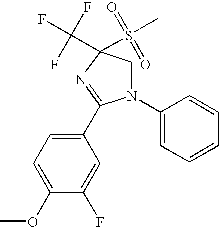 |
| | 2-(3-fluoro-4-methoxyphenyl)-1-[4- |
| | (methylsulfonyl)phenyl-4- |
| | (trifluoromethyl)]-1H-imidazole; |
| B-141 | 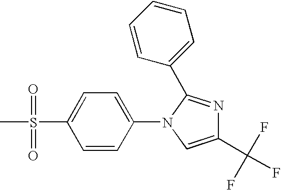 |
| | 1-[4-(methylsulfonyl)phenyl]-2-phenyl-4- |
| | trifluoromethyl-1H-imidazole; |
| B-142 | 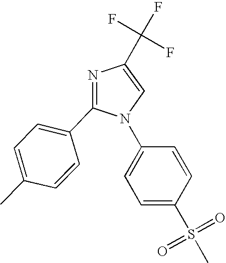 |
| | 2-(4-methylphenyl)-1-[4-(methylsulfonyl) |
| | phenyl]-4-trifluoromethyl-1H-imidazole; |
| B-143 | 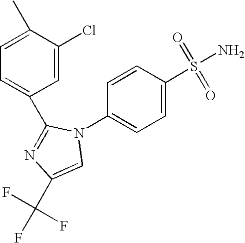 |
| | 4-[2-(3-chloro-4-methylphenyl)-4- |
| | (trifluoromethyl)-1H-imidazol-1- |
| | yl]benzenesulfonamide; |
| B-144 | 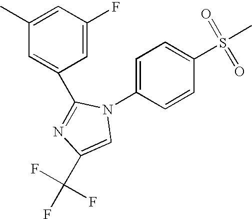 |
| | 2-(3-fluoro-5-methylphenyl)-1-[4- |
| | (methylsulfonyl)phenyl]- |
| | 4-(trifluoromethyl)-1H-imidazole; |
| B-145 | 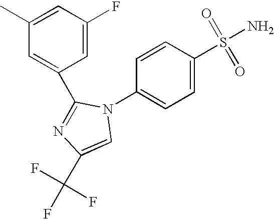 |
| | 4-[2-(3-fluoro-5-methylphenyl)-4- |
| | (trifluoromethyl-1H-imidazole-1- |
| | yl]benzenesulfonamide; |
| B-146 | 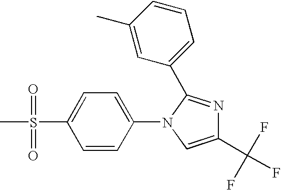 |
| | 2-(3-methylphenyl)-1-[4-(methylsulfonyl) |
| | phenyl]-4-trifluoromethyl-1H-imidazole; |
| B-147 | 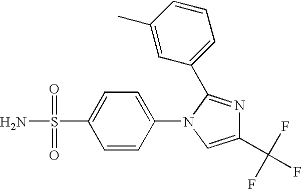 |
| | 4-[2-(3-methylphenyl)-4-trifluoromethyl- |
| | 1H-imidazol-1-yl]benzenesulfonamide |
| B-148 | 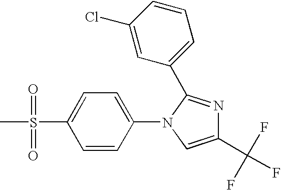 |
| | 1-[4-(methylsulfonyl)phenyl]-2-(3- |
| | chlorophenyl)-4-trifluoromethyl-1H-imidazole |
| B-149 | 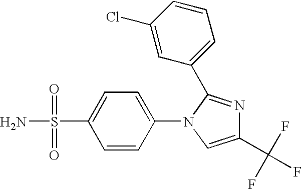 |
| | 4-[2-(3-chlorophenyl)-4-trifluoromethyl-1H- |
| | imidazol-1-yl]benzenesulfonamide |
| B-150 | 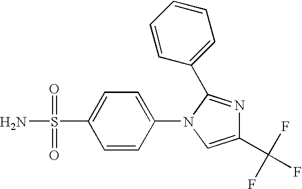 |
| | 4-[2-phenyl-4-trifluoromethyl-1H-imidazol-1- |
| | yl]benzenesulfonamide; |
| B-151 | 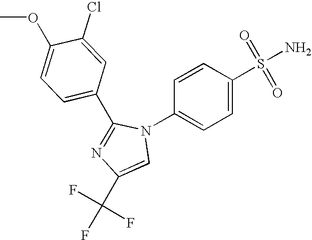 |
| | 4-[2-(4-methoxy-3-chlorophenyl)-4- |
| | trifluoromethyl-1H-imidazol-1- |
| | yl]benzenesulfonamide; |
| B-152 | 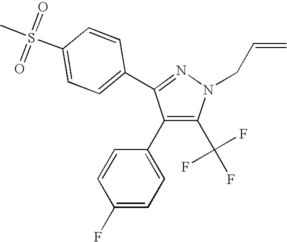 |
| | 1-allyl-4-(4-fluorophenyl)-3-[4- |
| | (methylsulfonyl)phenyl]-5- |
| | (trifluoromethyl)-1H-pyrazole; |
| B-153 | 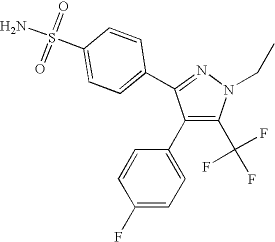 |
| | 4-[1-ethyl-4-(4-fluorophenyl)-5- |
| | (trifluoromethyl)-1H-pyrazol-3-yl] |
| | benzenesulfonamide; |
| B-154 | 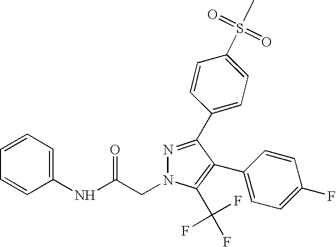 |
| | N-phenyl-[4-(4-fluorophenyl)-3-[4- |
| | (methylsulfonyl)phenyl]- |
| | 5-(trifluoromethyl)-1H-pyrazol-1-yl]acetamide; |
| B-155 | 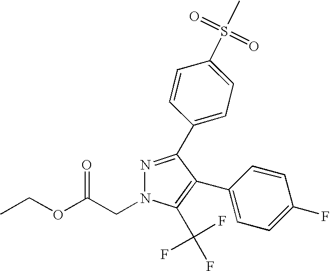 |
| | ethyl[4-(4-fluorophenyl)-3-[4- |
| | (methylsulfonyl)phenyl]-5-(trifluoromethyl)- |
| | 1H-pyrazol-1-yl]acetate; |
| B-156 | 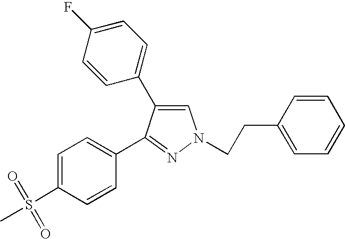 |
| | 4-(4-fluorophenyl)-3-[4-(methylsulfonyl) |
| | phenyl]-1-(2-phenylethyl)-1H-pyrazole; |
| B-157 | 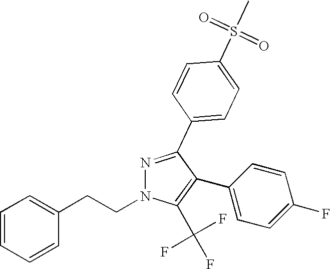 |
| | 4-(4-fluorophenyl)-3-[4-(methylsulfonyl) |
| | phenyl]-1-(2-phenylethyl )-5- |
| | (trifluoromethyl)pyrazole; |
| B-158 | 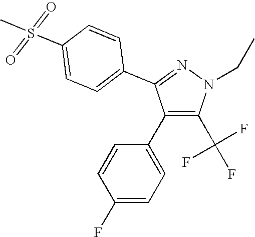 |
| | 1-ethyl-4-(4-fluorophenyl)-3-[4- |
| | methylsulfonyl)phenyl]-5-(trifluoromethyl)- |
| | 1H-pyrazole; |
| B-159 | 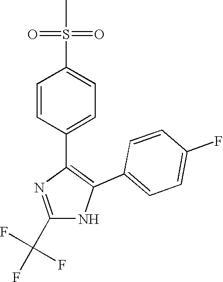 |
| | 5-(4-fluorophenyl)-4-(4-methylsulfonylphenyl)- |
| | 2-trifluoromethyl-1H-imidazole; |
| B-160 | 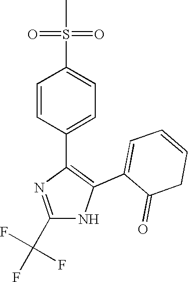 |
| | 4-[4-(methylsulfonyl)phenyl]-5-(2- |
| | thiophenyl)-2-(trifluoromethyl)-1H-imidazole; |
| B-161 | 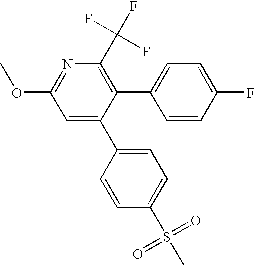 |
| | 5-(4-fluorophenyl)-2-methoxy-4-[4- |
| | (methylsulfonyl)phenyl]-6-(trifluoromethyl)pyridine; |
| B-162 | 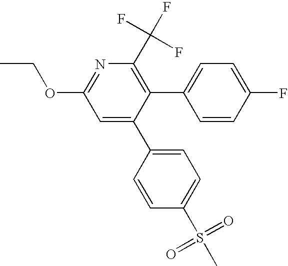 |
| | 2-ethoxy-5-(4-fluorophenyl)-4-[4- |
| | (methylsulfonyl)phenyl]-6- |
| | (trifluoromethyl)pyridine; |
| B-163 | 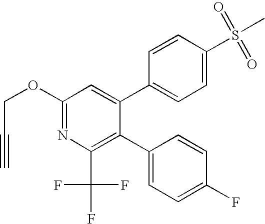 |
| | 5-(4-fluorophenyl)-4-[4-(methylsulfonyl) |
| | phenyl]-2-(2-propynyloxy)-6- |
| | (trifluoromethyl)pyridine; |
| B-164 | 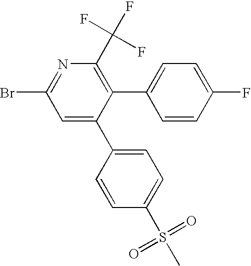 |
| | 2-bromo-5-(4-fluorophenyl)-4-[4- |
| | (methylsulfonyl)phenyl]-6- |
| | (trifluoromethyl)pyridine; |
| B-165 | 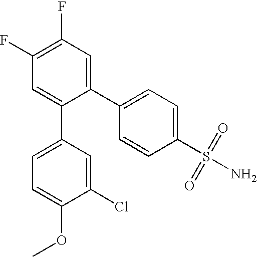 |
| | 4-[2-(3-chloro-4-methoxyphenyl)-4,5- |
| | difluorophenyl]benzenesulfonamide; |
| B-166 | 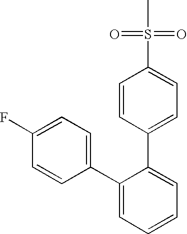 |
| | 1-(4-fluorophenyl)-2-[4-methylsulfonyl) |
| | phenyl]benzene; |
| B-167 | 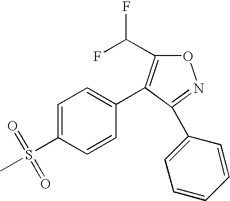 |
| | 5-difluoromethyl-4-(4-methylsulfonylphenyl)- |
| | 3-phenylisoxazole; |
| B-168 | 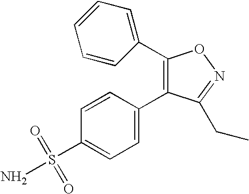 |
| | 4-[3-ethyl-5-phenylisoxazol-4-yl] |
| | benzenesulfonamide; |
| B-169 | 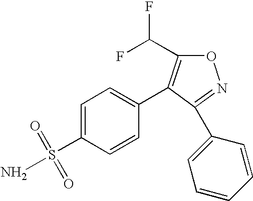 |
| | 4-[5-difluoromethyl-3-phenylisoxazol- |
| | 4-yl]benzenesulfonamide; |
| B-170 | 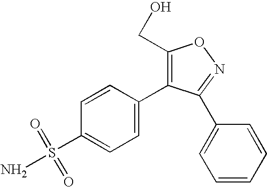 |
| | 4-[5-hydroxymethyl-3-phenylisoxazol- |
| | 4-yl]benzenesulfonamide; |
| B-171 | 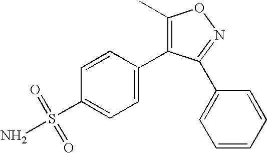 |
| | 4-[5-methyl-3-phenyl-isoxazol-4- |
| | yl]benzenesulfonamide; |
| B-172 | 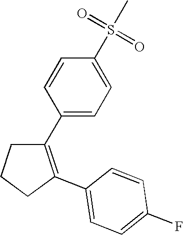 |
| | 1-[2-(4-fluorophenyl)cyclopenten-1- |
| | yl]-4-(methylsulfonyl)benzene; |
| B-173 | 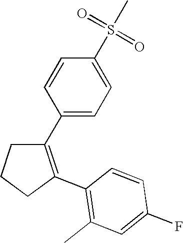 |
| | 1-[2-(4-fluoro-2-methylphenyl)cyclopenten- |
| | 1-yl]-4-(methylsulfonyl)benzene; |
| B-174 | 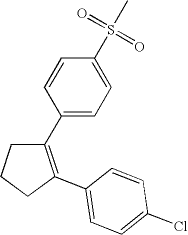 |
| | 1-[2-(4-chlorophenyl)cyclopenten-1- |
| | yl]-4-(methylsulfonyl)benzene; |
| B-175 | 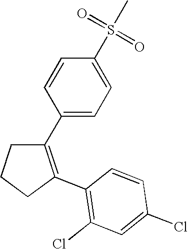 |
| | 1-[2-(2,4-dichlorophenyl)cyclopenten-1- |
| | yl]-4-(methylsulfonyl)benzene |
| B-176 | 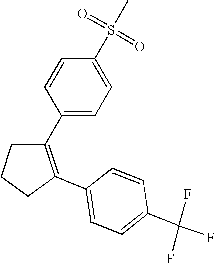 |
| | 1-[2-(4-trifloromethylphenyl)cyclopenten- |
| | 1-yl)-4-(methylsulfonyl)benzene; |
| B-177 | 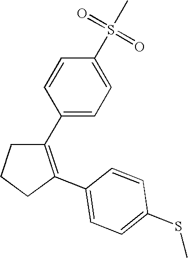 |
| | 1-[2-(4-methylthiophenyl)cyclopenten-1- |
| | yl]-4-(methylsulfonyl)benzene; |
| B-178 | 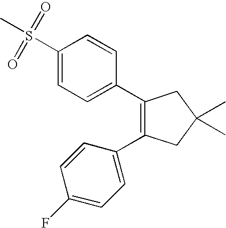 |
| | 1-[2-(4-fluorophenyl)-4,4-dimethylcyclopenten- |
| | 1-yl]-4-(methylsulfonyl)benzene; |
| B-179 | 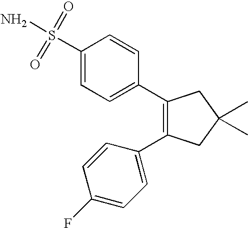 |
| | 4-[2-(4-fluorophenyl)-4,4- |
| | dimethylcyclopenten-1-yl]benzenesulfonamide; |
| B-180 | 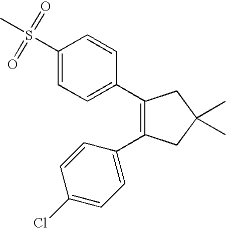 |
| | 1-[2-(3-chlorophenyl)-4,4- |
| | dimethylcyclopenten-1-yl]-4- |
| | (methylsulfonyl)benzene |
| B-181 | 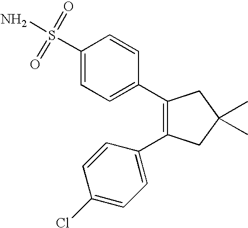 |
| | 4-[2-(4-chlorophenyl)-4,4- |
| | dimethylcyclopenten-1-yl]benzenesulfonamide; |
| B-182 | 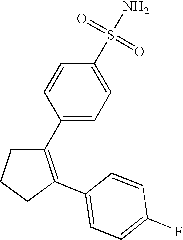 |
| | 4-[2-(4-fluorophenyl)cyclopenten-1- |
| | yl]benzenesulfonamide; |
| B-183 | 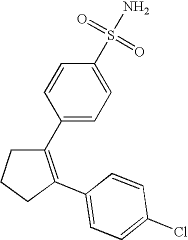 |
| | 4-[2-(4-chlorophenyl)cyclopenten-1- |
| | yl]benzenesulfonamide; |
| B-184 | 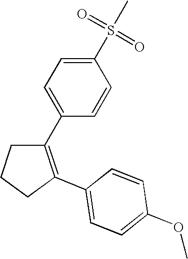 |
| | 1-[2-(4-methoxyphenyl)cyclopenten-1- |
| | yl]-4-(methylsulfonyl)benzene; |
| B-185 | 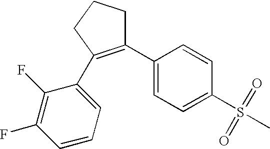 |
| | 1-[2-(2,3-difluorophenyl)cyclopenten- |
| | 1-yl]-4-(methylsulfonyl)benzene; |
| B-186 | 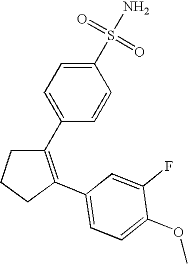 |
| | 4-[2-(3-fluoro-4-methoxyphenyl)cyclopenten- |
| | 1-yl]benzenesulfonamide; |
| B-187 | 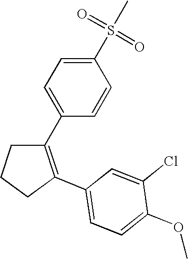 |
| | 1-[2-(3-chloro-4-methoxyphenyl)cyclopenten- |
| | 1-yl]-4-(methylsulfonyl)benzene; |
| B-188 | 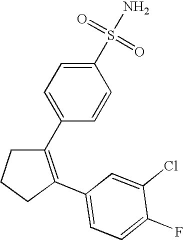 |
| | 4-[2-(3-chloro-4-fluorophenyl)cyclopenten- |
| | 1-yl]benzenesulfonamide; |
| B-189 | 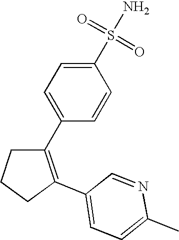 |
| | 4-[2-(2-methylpyridin-5-yl)cyclopenten-1- |
| | yl]benzenesulfonamide; |
| B-190 | 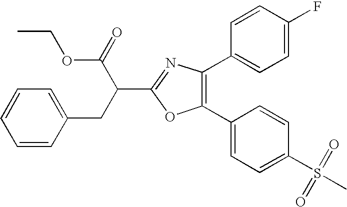 |
| | ethyl 2-[4-(4-fluorophenyl)-5-[4- |
| | (methylsulfonyl)phenyl]oxazol-2- |
| | yl]-2-benzyl-acetate; |
| B-191 | 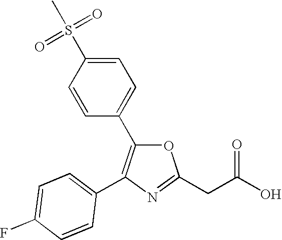 |
| | 2-[4-(4-fluorophenyl)-5-[4- |
| | (methylsulfonyl)phenyl]oxazol-2- |
| | yl]acetic acid; |
| B-192 | 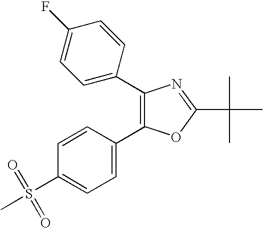 |
| | 2-(tert-butyl)-4-(4-fluorophenyl)-5- |
| | [4-(methylsulfonyl)phenyl]oxazole; |
| B-193 | 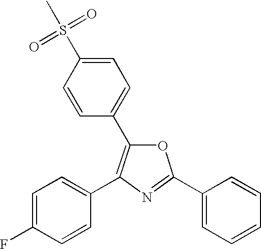 |
| | 4-(4-fluorophenyl)-5-[4-(methylsulfonyl) |
| | phenyl]-2-phenyloxazole; |
| B-194 | 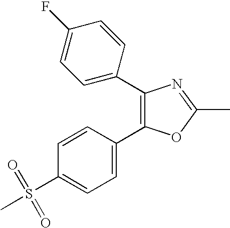 |
| | 4-(4-fluorophenyl)-2-methyl-5-[4- |
| | (methylsulfonyl)phenyl]oxazole; |
| B-195 | 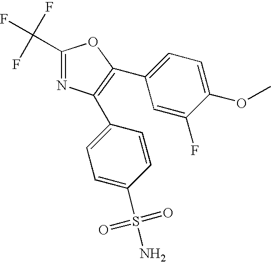 |
| | 4-[5-(3-fluoro-4-methoxyphenyl)-2- |
| | trifluoromethyl-4-oxazolyl]benzenesulfonamide; |
| B-196 | 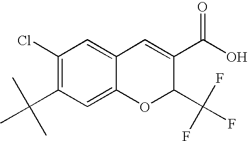 |
| | 6-chloro-7-(1,1-dimethylethyl)-2- |
| | trifluoromethyl-2H-1-benzopyran-3- |
| | carboxylic acid; |
| B-197 | 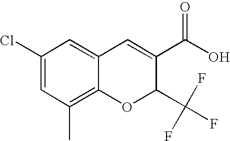 |
| | 6-chloro-8-methyl-2-trifluoromethyl-2H- |
| | 1-benzopyran-3-carboxylic acid; |
| B-198 | 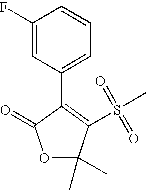 |
| | 5,5-dimethyl-3-(3-fluorophenyl)-4- |
| | methylsulfonyl-2(5H)-furanone; |
| B-199 | 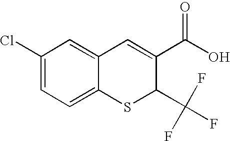 |
| | 6-chloro-2-trifluoromethyl-2H-1- |
| | benzothiopyran-3-carboxylic acid; |
| B-200 | 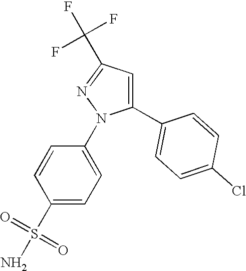 |
| | 4-[5-(4-chlorophenyl)-3-(trifluoromethyl)- |
| | 1H-pyrazol-1-yl]benzenesulfonamide; |
| B-201 | 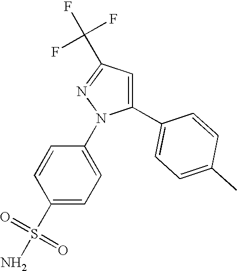 |
| | 4-[5-(4-methylphenyl)-3-(trifluoromethyl)- |
| | 1H-pyrazol-1-yl]benzenesulfonamide; |
| B-202 | 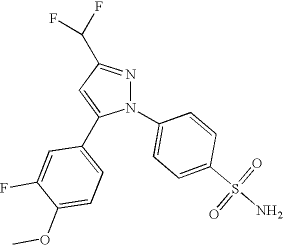 |
| | 4-[5-(3-fluoro-4-methoxyphenyl)-3- |
| | (difluoromethyl)-1H-pyrazol-1- |
| | yl]benzenesulfonamide; |
| B-203 | 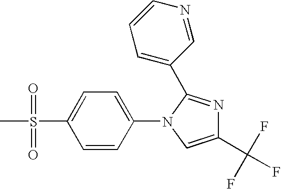 |
| | 3-[1-[4-(methylsulfonyl)phenyl]-4- |
| | trifluoromethyl-1H-imidazol-2-yl]pyridine; |
| B-204 | 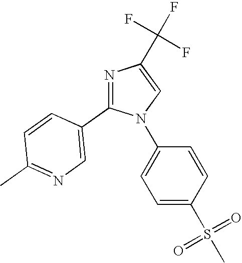 |
| | 2-methyl-5-[1-[4-(methylsulfonyl)phenyl]-4- |
| | trifluoromethyl-1H-imidazol-2-yl]pyridine; |
| B-205 | 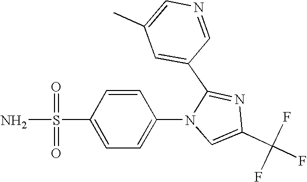 |
| | 4-[2-(5-methylpyridin-3-yl)-4-(trifluoromethyl)- |
| | 1H-imidazol-1-yl]benzenesulfonamide; |
| B-206 | 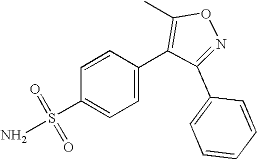 |
| | 4-[5-methyl-3-phenylisoxazol-4-yl] |
| | benzenesulfonamide; |
| B-207 | 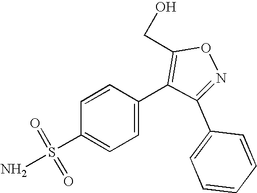 |
| | 4-[5-hydroxymethyl-3-phenylisoxazol-4- |
| | yl]benzenesulfonamide; |
| B-208 | 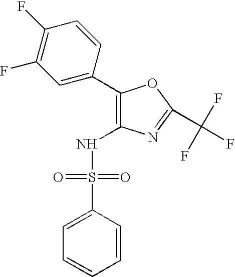 |
| | [2-trifluoromethyl-5-(3,4-difluorophenyl)- |
| | 4-oxazolyl]benzenesulfonamide; |
| B-209 | 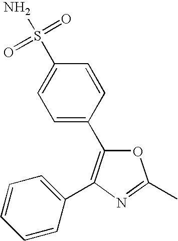 |
| | 4-[2-methyl-4-phenyl-5-oxazolyl] |
| | benzenesulfonamide; |
| B-210 | 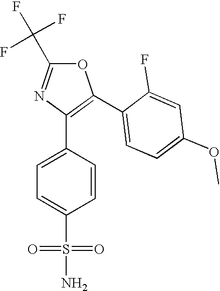 |
| | 4-[5-(2-fluoro-4-methoxyphenyl)-2- |
| | trifluoromethyl-4-oxazolyl] |
| | benzenesulfonamide; |
| B-211 | 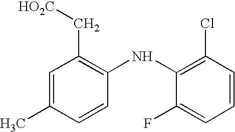 |
| B-212 | 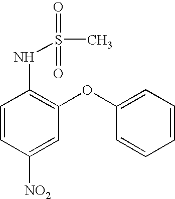 |
| | N-(4-nitro-2-phenoxy-phenyl)- |
| | methanesulfonamide or Nimesulide |
| B-213 | 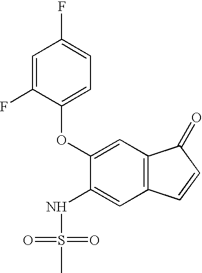 |
| | N-[6-(2,4-difluoro-phenoxy)-1-oxo-inden-5- |
| | yl]-methanesulfonamide or Flosulide |
| B-214 | 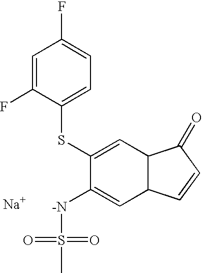 |
| | N-[6-(2,4-difluoro-phenylsulfanyl)-1-oxo- |
| | 1H-inden-5-yl]-methanesulfonamide, |
| | soldium salt, or L-745337 |
| B-215 | 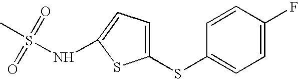 |
| | N-[5-(4-fluoro-phenylsulfanyl)-thiophen-2- |
| | yl]-methanesulfonamide or RWJ-63556 |
| B-216 | 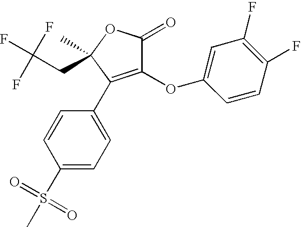 |
| | 3-(3,4-difluoro-phenoxy)-4-(4-methanesulfonyl- |
| | phenyl)-5-methyl-5-(2,2,2-trifluoro-ethyl)-5H- |
| | furan-2-one or L-784512 |
| B-217 | 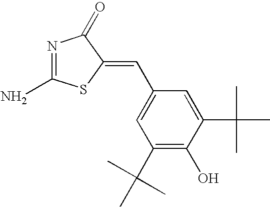 |
| | (5Z)-2-amino-5-[[3,5-bis(1,1-dimethylethyl)-4- |
| | hydroxyphenyl]methylene]-4(5H)- |
| | thiazolone or Darbufelone |
| B-218 | CS-502 |
| B-219 | LAS-34475 |
| B-220 | LAS-34555 |
| B-221 | S-33516 |
| B-222 | SD-8381 |
| B-223 | L-783003 |
| B-224 | 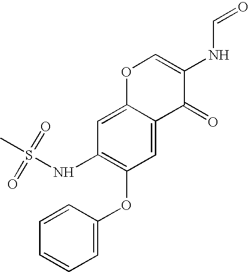 |
| | N-[3-(formylamino)-4-oxo-6-phenoxy-4H-1- |
| | benzopyran-7-yl]-methanesulfonanxide or T614 |
| B-225 | D-1367 |
| B-226 | L-748731 |
| B-227 | 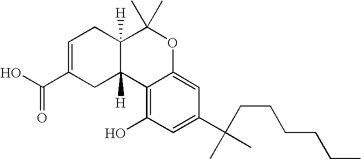 |
| | (6aR,10aR)-3-(1,1-dimethylheptyl)-6a,7,10,10a- |
| | tetrahydro-1-hydroxy-6,6-dimethyl-6H- |
| | dibenzo[b,d]pyran-9- |
| | carboxylic acid or CT3 |
| B-228 | CGP-28238 |
| B-229 | 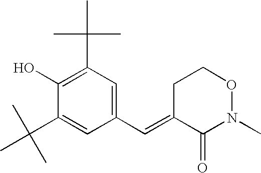 |
| | 4-[[3,5-bis(1,1-dimethylethyl)-4- |
| | hydroxyphenyl]methylene] |
| | dihydro-2-methyl-2H-1,2-oxazin-3(4H)- |
| | one or BF-389 |
| B-230 | GR-253035 |
| B-231 | 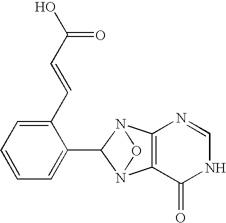 |
| | 2-(6-dioxo-9H-purin-8-yl)cinnamic acid |
| B-232 | S-2474 |
| B-233 | 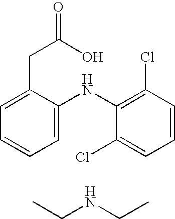 |
| B-234 | 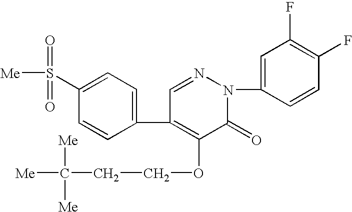 |
| B-235 | 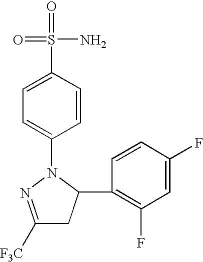 |
| B-236 | 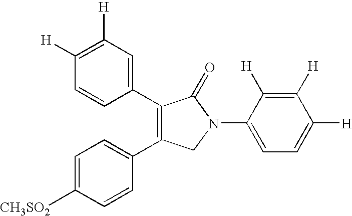 |
| B-237 | 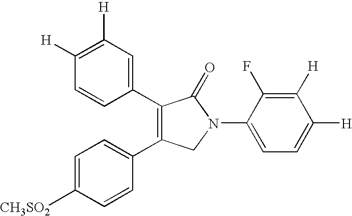 |
| B-238 | 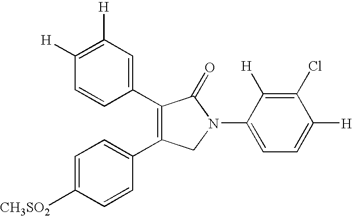 |
| B-239 | 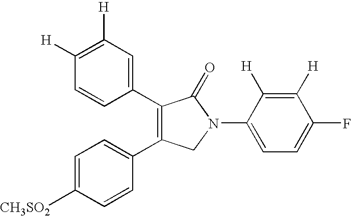 |
| B-240 | 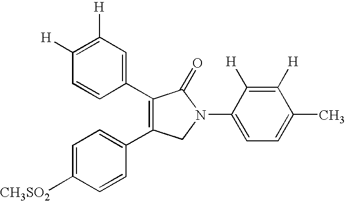 |
| B-241 | 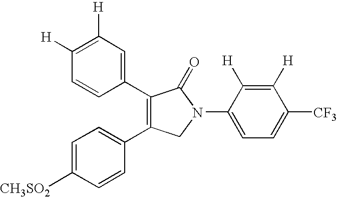 |
| B-242 | 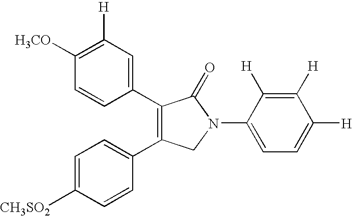 |
| B-243 | 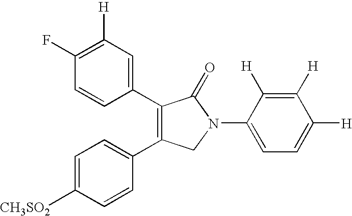 |
| B-244 | 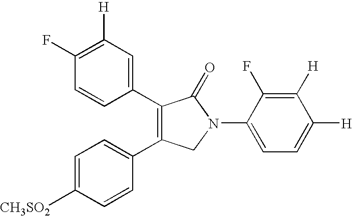 |
| B-245 | 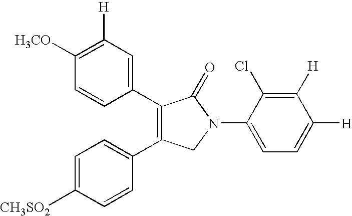 |
| B-246 | 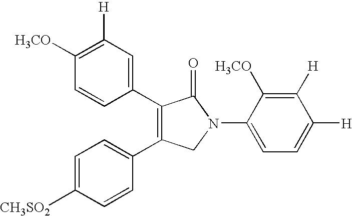 |
| B-247 | 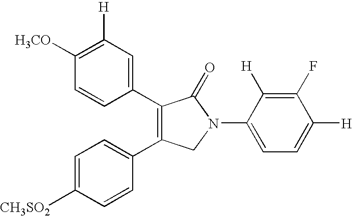 |
| B-248 | 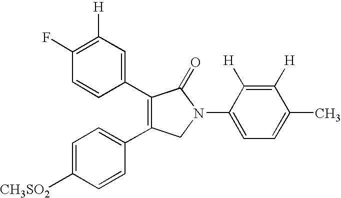 |
| B-249 | 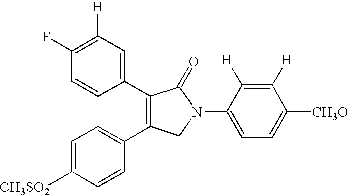 |
| B-250 | 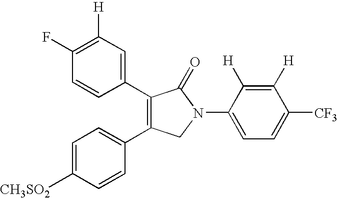 |
| B-251 | 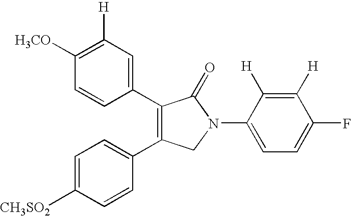 |
| B-252 | 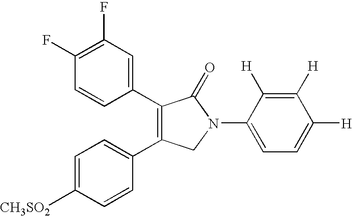 |

The cyclooxygenase-2 selective inhibitor employed in the present invention can exist in tautomeric, geometric or stereoisomeric forms. Generally speaking, suitable cyclooxygenase-2 selective inhibitors that are in tautomeric, geometric or stereoisomeric forms are those compounds that inhibit cyclooxygenase-2 activity by about 25%, more typically by about 50%, and even more typically, by about 75% or more when present at a concentration of 100 μM or less. The present invention contemplates all such compounds, including cis- and trans-geometric isomers, E- and Z-geometric isomers, R- and S-enantiomers, diastereomers, d-isomers, 1-isomers, the racemic mixtures thereof and other mixtures thereof. Pharmaceutically acceptable salts of such tautomeric, geometric or stereoisomeric forms are also included within the invention. The terms “cis” and “trans”, as used herein, denote a form of geometric isomerism in which two carbon atoms connected by a double bond will each have a hydrogen atom on the same side of the double bond (“cis”) or on opposite sides of the double bond (“trans”). Some of the compounds described contain alkenyl groups, and are meant to include both cis and trans or “E” and “Z” geometric forms. Furthermore, some of the compounds described contain one or more stereocenters and are meant to include R, S, and mixtures or R and S forms for each stereocenter present.

The cyclooxygenase-2 selective inhibitors utilized in the present invention may be in the form of free bases or pharmaceutically acceptable acid addition salts thereof. The term “pharmaceutically-acceptable salts” are salts commonly used to form alkali metal salts and to form addition salts of free acids or free bases. The nature of the salt may vary, provided that it is pharmaceutically acceptable. Suitable pharmaceutically acceptable acid addition salts of compounds for use in the present methods may be prepared from an inorganic acid or from an organic acid. Examples of such inorganic acids are hydrochloric, hydrobromic, hydroiodic, nitric, carbonic, sulfuric and phosphoric acid. Appropriate organic acids may be selected from aliphatic, cycloaliphatic, aromatic, araliphatic, heterocyclic, carboxylic and sulfonic classes of organic acids, examples of which are formic, acetic, propionic, succinic, glycolic, gluconic, lactic, malic, tartaric, citric, ascorbic, glucuronic, maleic, fumaric, pyruvic, aspartic, glutamic, benzoic, anthranilic, mesylic, 4-hydroxybenzoic, phenylacetic, mandelic, embonic (pamoic), methanesulfonic, ethanesulfonic, benzenesulfonic, pantothenic, 2-hydroxyethanesulfonic, toluenesulfonic, sulfanilic, cyclohexylaminosulfonic, stearic, algenic, hydroxybutyric, salicylic, galactaric and galacturonic acid. Suitable pharmaceutically-acceptable base addition salts of compounds of use in the present methods include metallic salts made from aluminum, calcium, lithium, magnesium, potassium, sodium and zinc or organic salts made from N,N′-dibenzylethylenediamine, chloroprocaine, choline, diethanolamine, ethylenediamine, meglumine (N-methylglucamine) and procaine. All of these salts may be prepared by conventional means from the corresponding compound by reacting, for example, the appropriate acid or base with the compound of any Formula set forth herein.

The cyclooxygenase-2 selective inhibitors of the present invention can be formulated into pharmaceutical compositions and administered by a number of different means that will deliver a therapeutically effective dose. Such compositions can be administered orally, parenterally, by inhalation spray, rectally, intradermally, transdermally, or topically in dosage unit formulations containing conventional nontoxic pharmaceutically acceptable carriers, adjuvants, and vehicles as desired. Topical administration may also involve the use of transdermal administration such as transdermal patches or iontophoresis devices. The term parenteral as used herein includes subcutaneous, intravenous, intramuscular, or intrasternal injection, or infusion techniques. Formulation of drugs is discussed in, for example, Hoover, John E., Remington's Pharmaceutical Sciences, Mack Publishing Co., Easton, Pa. (1975), and Liberman, H. A. and Lachman, L., Eds., Pharmaceutical Dosage Forms, Marcel Decker, New York, N.Y. (1980).

Injectable preparations, for example, sterile injectable aqueous or oleaginous suspensions, can be formulated according to the known art using suitable dispersing or wetting agents and suspending agents. The sterile injectable preparation may also be a sterile injectable solution or suspension in a nontoxic parenterally acceptable diluent or solvent. Among the acceptable vehicles and solvents that may be employed are water, Ringer's solution, and isotonic sodium chloride solution. In addition, sterile, fixed oils are conventionally employed as a solvent or suspending medium. For this purpose, any bland fixed oil may be employed, including synthetic mono- or diglycerides. In addition, fatty acids such as oleic acid are useful in the preparation of injectables. Dimethyl acetamide, surfactants including ionic and non-ionic detergents, and polyethylene glycols can be used. Mixtures of solvents and wetting agents such as those discussed above are also useful.

Suppositories for rectal administration of the compounds discussed herein can be prepared by mixing the active agent with a suitable non-irritating excipient such as cocoa butter, synthetic mono-, di-, or triglycerides, fatty acids, or polyethylene glycols which are solid at ordinary temperatures but liquid at the rectal temperature, and which will therefore melt in the rectum and release the drug.

Solid dosage forms for oral administration may include capsules, tablets, pills, powders, and granules. In such solid dosage forms, the compounds are ordinarily combined with one or more adjuvants appropriate to the indicated route of administration. If administered per os, the compounds can be admixed with lactose, sucrose, starch powder, cellulose esters of alkanoic acids, cellulose alkyl esters, talc, stearic acid, magnesium stearate, magnesium oxide, sodium and calcium salts of phosphoric and sulfuric acids, gelatin, acacia gum, sodium alginate, polyvinylpyrrolidone, and/or polyvinyl alcohol, and then tableted or encapsulated for convenient administration. Such capsules or tablets can contain a controlled-release formulation as can be provided in a dispersion of active compound in hydroxypropylmethyl cellulose. In the case of capsules, tablets, and pills, the dosage forms can also comprise buffering agents such as sodium citrate, or magnesium or calcium carbonate or bicarbonate. Tablets and pills can additionally be prepared with enteric coatings.

For therapeutic purposes, formulations for parenteral administration can be in the form of aqueous or non-aqueous isotonic sterile injection solutions or suspensions. These solutions and suspensions can be prepared from sterile powders or granules having one or more of the carriers or diluents mentioned for use in the formulations for oral administration. The compounds can be dissolved in water, polyethylene glycol, propylene glycol, ethanol, corn oil, cottonseed oil, peanut oil, sesame oil, benzyl alcohol, sodium chloride, and/or various buffers. Other adjuvants and modes of administration are well and widely known in the pharmaceutical art.

Liquid dosage forms for oral administration can include pharmaceutically acceptable emulsions, solutions, suspensions, syrups, and elixirs containing inert diluents commonly used in the art, such as water. Such compositions can also comprise adjuvants, such as wetting agents, emulsifying and suspending agents, and sweetening, flavoring, and perfuming agents.

The amount of active ingredient that can be combined with the carrier materials to produce a single dosage of the cyclooxygenase-2 selective inhibitor will vary depending upon the patient and the particular mode of administration. In general, the pharmaceutical compositions may contain a cyclooxygenase-2 selective inhibitor in the range of about 0.1 to 2000 mg, more typically, in the range of about 0.5 to 500 mg and still more typically, between about 1 and 200 mg. A daily dose of about 0.01 to 100 mg/kg body weight, or more typically, between about 0.1 and about 50 mg/kg body weight and even more typically, from about 1 to 20 mg/kg body weight, may be appropriate. The daily dose is generally administered in one to about four doses per day.

In one embodiment, when the cyclooxygenase-2 selective inhibitor comprises rofecoxib, it is typical that the amount used is within a range of from about 0.15 to about 1.0 mg/day/kg, and even more typically, from about 0.18 to about 0.4 mg/day/kg.

In still another embodiment, when the cyclooxygenase-2 selective inhibitor comprises etoricoxib, it is typical that the amount used is within a range of from about 0.5 to about 5 mg/day/kg, and even more typically, from about 0.8 to about 4 mg/day/kg.

Further, when the cyclooxygenase-2 selective inhibitor comprises celecoxib, it is typical that the amount used is within a range of from about 1 to about 20 mg/day/kg, even more typically, from about 1.4 to about 8.6 mg/day/kg, and yet more typically, from about 2 to about 3 mg/day/kg.

When the cyclooxygenase-2 selective inhibitor comprises valdecoxib, it is typical that the amount used is within a range of from about 0.1 to about 5 mg/day/kg, and even more typically, from about 0.8 to about 4 mg/day/kg.

In a further embodiment, when the cyclooxygenase-2 selective inhibitor comprises parecoxib, it is typical that the amount used is within a range of from about 0.1 to about 5 mg/day/kg, and even more typically, from about 1 to about 3 mg/day/kg.

Those skilled in the art will appreciate that dosages may also be determined with guidance from Goodman & Goldman's The Pharmacological Basis of Therapeutics, Ninth Edition (1996), Appendix II, pp. 1707-1711 and from Goodman & Goldman's The Pharmacological Basis of Therapeutics, Tenth Edition (2001), Appendix II, pp. 475-493.

5-HT_{1B/1D} Agonists

In addition to a cyclooxygenase-2 selective inhibitor, the composition of the invention also comprises a 5-HT_{1B/1D} agonist.

In one embodiment, the 5-HT_{1B/1D} agonist is a triptan. Triptans activate the 5-HT_{1B/1D} serotonin receptor sites. In yet another embodiment, the triptan is eletriptan, frovatriptan, naratriptan, rizatriptan, sumatriptan or zolmitriptan. In a further embodiment, the triptan is preferably eletriptan. Eletriptan is particularly suitable for use in the present invention because studies to-date show that eletriptan has a greater effectiveness at one and two hours after administration than sumatriptan. Eletriptan and the other triptans may be obtained from commercial sources.

The 5-HT_{1B/1D} agonist may be administrated to a subject by any suitable means generally known in the art. In another embodiment, preferably, the 5-HT_{1B/1D} agonist is administered orally or via bolus injection. The bolus injection can take place intravenously, intramuscularly or also subcutaneously. In yet another embodiment, preferably, the bolus injection is administered as an intravenous injection.

There are four phases to a migraine attack. Generally, there is the prodrome, the auras, the migraine attack and the postdrome. The patient may be in need of pain relief in any one of these phases and administration of certain 5-HT_{1B/1D} agonists is most beneficial during certain phases.

Generally speaking, the history of the patient, the severity of the attack, and whether the 5-HT_{1B/1D} agonist is administered during an acute migraine attack or for prevention of a migraine attack will dictate the most preferred method of administration and dosing regimen. For example, when the patient is having an acute migraine attack, the 5-HT_{1B/1D} agonist should be selected from the triptans. The method of administration for the triptans is dependent on how long the administration of the 5-HT_{1B/1D} agonist is after the beginning of the headache phase and the severity of the headache. Parenteral administration is indicated when the headache is severe and the time is after the beginning of the headache phase. Alternatively, oral triptans or anti-inflammatories or a combination are indicated when the headache is not severe and the subject is in the prodrome phase of the migraine.

The triptans generally are administered in the form of an oral tablet. However, sumatriptan may be administered parenterally or in a nasal spray form. The typical dose for eletriptan is an initial 40 mg dose during the headache phase and, if the headache recurs within 24 hours, the dose may be repeated. The maximum daily dose of eletriptan is 80 mg. The dose for frovatriptan is 2.5 mg initially, with a second dose given, if needed, after 2 hours. The maximum daily dose of frovatriptan is 7.5 mg.

The dose for naratriptan is 2.5 mg initially, with a second dose after 4 hours if needed. The maximum daily dose of naratriptan is 5 mg. The dose for rizatriptan is 10 mg initially, with a second dose after 2 hours if needed. The maximum daily dose of rizatriptan is 20 mg in 24 hours. The dose for zolmitriptan is 2.5 mg initially, with a second dose after 2 hours if needed. The maximum daily dose of zolmitriptan is 15 mg in 24 hours.

The timing of the administration of the 5-HT_{1B/1D} agonist can vary. For example, the 5-HT_{1B/1D} agonist can be administered beginning at a time prior to, at the time of, or at a time after the onset of migraine. Administration can be by a single dose, or the 5-HT_{1B/1D} agonist is given over an extended period. The administration of the 5-HT_{1B/1D} agonist extend for a period after the onset of migraine. In one embodiment, administration is continued for six months following the onset of migraine. In other embodiments, administration of the 5-HT_{1B/1D} agonist is continued for 1 week, 2 weeks, 1 month, 3 months, 9 months, or one year after the onset of migraine.

Equally, the timing of the administration of the cyclooxygenase-2 selective inhibitor can also vary. For example, the cyclooxygenase-2 selective inhibitor can be administered beginning at a time prior to, at the time of, or at a time after the onset of migraine. Administration can be by a single dose, or more preferably the cyclooxygenase-2 selective inhibitor is given over an extended period. It is preferred that administration of the cyclooxygenase-2 selective inhibitor extend for a period after the onset of migraine. In one embodiment, administration is continued for six months following the onset of migraine. In other embodiments, administration of the cyclooxygenase-2 selective inhibitor is continued for 1 week, 2 weeks, 1 month, 3 months, 9 months, or one year after the onset of migraine.

The timing of the administration of the cyclooxygenase-2 selective inhibitor in relation to the administration of the 5-HT_{1B/1D} agonist may also vary from subject to subject and depend upon the type of migraine being treated. In one embodiment of the invention, the cyclooxygenase-2 selective inhibitor and 5-HT_{1B/1D} agonist may be administered substantially simultaneously, meaning that both agents may be administered to the subject at approximately the same time. For example, the cyclooxygenase-2 selective inhibitor is administered during a continuous period beginning on the same day as the beginning of the 5-HT_{1B/1D} agonist and extending to a period after the end of the 5-HT_{1B/1D} agonist. Alternatively, the cyclooxygenase-2 selective inhibitor and 5-HT_{1B/1D} agonist may be administered sequentially, meaning that they are administered at separate times during separate treatments. In one embodiment, for example, the cyclooxygenase-2 selective inhibitor is administered during a continuous period beginning prior to administration of the 5-HT_{1B/1D} agonist and ending after administration of the 5-HT_{1B/1D} agonist. Of course, it is also possible that the cyclooxygenase-2 selective inhibitor may be administered either more or less frequently than the 5-HT_{1B/1D} agonist. One skilled in the art can readily design suitable treatment regiments for a particular subject depending on the particular type of migraine being treated. Moreover, it will be apparent to those skilled in the art that it is possible, and perhaps desirable, to combine various times and methods of administration in the practice of the present invention.

Combination Therapies

Generally speaking, it is contemplated that the composition employed in the practice of the invention may include one or more of any of the cyclooxygenase-2 selective inhibitors detailed above in combination with one or more of any of the 5-HT_{1B/1D} agonists detailed above. By way of a non limiting example, Table 4 details a number of suitable combinations that are useful in the methods and compositions of the current invention. The combination may also include an isomer, a pharmaceutically acceptable salt, ester, or prodrug of any of the cyclooxygenase-2 selective inhibitors or the 5-HT_{1B/1D} agonists in Table 4.

TABLE 4

| Cyclooxygenase-2 Selective Inhibitor | 5-HT 1B/1D Agonist |
|---|---|
| a compound having formula I | triptan |
| a compound having formula I | eletriptan |
| a compound having formula I | frovatriptan |
| a compound having formula I | naratriptan |
| a compound having formula I | rizatriptan |
| a compound having formula I | sumatriptan |
| a compound having formula I | zolmitriptan |
| a compound having formula II | triptan |
| a compound having formula II | eletriptan |
| a compound having formula II | frovatriptan |
| a compound having formula II | naratriptan |
| a compound having formula II | rizatriptan |
| a compound having formula II | sumatriptan |
| a compound having formula II | zolmitriptan |
| a compound having formula III | triptan |
| a compound having formula III | eletriptan |
| a compound having formula III | frovatriptan |
| a compound having formula III | naratriptan |
| a compound having formula III | rizatriptan |
| a compound having formula III | sumatriptan |
| a compound having formula III | zolmitriptan |
| a compound having formula VI | triptan |
| a compound having formula VI | eletriptan |
| a compound having formula VI | frovatriptan |
| a compound having formula VI | naratriptan |
| a compound having formula VI | rizatriptan |
| a compound having formula VI | sumatriptan |
| a compound having formula VI | zolmitriptan |
| a compound having formula V | triptan |
| a compound having formula V | eletriptan |
| a compound having formula V | frovatriptan |
| a compound having formula V | naratriptan |
| a compound having formula V | rizatriptan |
| a compound having formula V | sumatriptan |
| a compound having formula V | zolmitriptan |

By way of further example, Table 5 details a number of suitable combinations that may be employed in the methods and compositions of the present invention. The combination may also include an isomer, a pharmaceutically acceptable salt, ester, or prodrug of any of the cyclooxygenase-2 selective inhibitors or 5-HT_{1B/1D} agonists listed in Table 5.

TABLE 5

| Cyclooxygenase-2 Selective Inhibitor | 5-HT 1B/1D Agonist |
|---|---|
| a compound selected from the group consisting of | triptan |
| B-1, B-2, B-3, B-4, B-5, B-6, B-7, B-8, B-9, | |
| B-10, B-11, B-12, B-13, B-14, B-15, B-16, B-17, | |
| B-18, B-19, B-20, B-21, B-22, B-23, B-24, B-25, | |
| B-26, B-27, B-28, B-29, B-30, B-31, B-32, | |
| B-33, B-34, B-35, B-36, B-37, B-38, B-39, B-40, | |
| B-41, B-42, B-43, B-44, B-45, B-46, B-47, B-48, | |
| B-49, B-50, B-51, B-52, B-53, B-54, B-55, B-56, | |
| B-57, B-58, B-59, B-60, B-61, B-62, B-63, B-64, | |
| B-65, B-66, B-67, B-68, B-69, B-70, B-71, B-72, | |
| B-73, B-74, B-75, B-76, B-77, B-78, B-79, B-80, | |
| B-81, B-82, B-83, B-84, B-85, B-86, B-87, B-88, | |
| B-89, B-90, B-91, B-92, B-93, B-94, B-95, B-96, | |
| B-97, B-98, B-99, B-100, B-101, B-102, B-103, | |
| B-104, B-105, B-106, B-107, B-108, B-109, | |
| B-110, B-111, B-112, B-113, B-114, B-115, | |
| B-116, B-117, B-118, B-119, B-120, B-121, | |
| B-122, B-123, B-124, B-125, B-126, B-127, | |
| B-128, B-129, B-130, B-131, B-132, B-133, | |
| B-134, B-135, B-136, B-137, B-138, B-139, | |
| B-140, B-141, B-142, B-143, B-144, B-145, | |
| B-146, B-147, B-148, B-149, B-150, B-151, | |
| B-152, B-153, B-154, B-155, B-156, B-157, | |
| B-158, B-159, B-160, B-161, B-162, B-163, | |
| B-164, B-165, B-166, B-167, B-168, B-169, | |
| B-170, B-171, B-172, B-173, B-174, B-175, | |
| B-176, B-177, B-178, B-179, B-180, B-181, | |
| B-182, B-183, B-184, B-185, B-186, B-187, | |
| B-188, B-189, B-190, B-191, B-192, B-193, | |
| B-194, B-195, B-196, B-197, B-198, B-199, | |
| B-200, B-201, B-202, B-203, B-204, B-205, | |
| B-206, B-207, B-208, B-209, B-210, B-211, | |
| B-212, B-213, B-214, B-215, B-216, B-217, | |
| B-218, B-219, B-220, B-221, B-222, B-223, | |
| B-224, B-225, B-226, B-227, B-228, B-229, | |
| B-230, B-231, B-232, B-233, B-234, B-235, | |
| B-236, B-237, B-238, B-239, B-240, B-241, | |
| B-242, B-243 B-244, B-245, B-246, B-247, | |
| B-248, B-249, B-250, B-251, B-252 | |
| a compound selected from the group consisting of | eletriptan |
| B-1, B-2, B-3, B-4, B-5, B-6, B-7, B-8, B-9, | |
| B-10, B-11, B-12, B-13, B-14, B-15, B-16, B-17, | |
| B-18, B-19, B-20, B-21, B-22, B-23, B-24, B-25, | |
| B-26, B-27, B-28, B-29, B-30, B-31, B-32, | |
| B-33, B-34, B-35, B-36, B-37, B-38, B-39, B-40, | |
| B-41, B-42, B-43, B-44, B-45, B-46, B-47, B-48, | |
| B-49, B-50, B-51, B-52, B-53, B-54, B-55, B-56, | |
| B-57, B-58, B-59, B-60, B-61, B-62, B-63, B-64, | |
| B-65, B-66, B-67, B-68, B-69, B-70, B-71, B-72, | |
| B-73, B-74, B-75, B-76, B-77, B-78, B-79, B-80, | |
| B-81, B-82, B-83, B-84, B-85, B-86, B-87, B-88, | |
| B-89, B-90, B-91, B-92, B-93, B-94, B-95, B-96, | |
| B-97, B-98, B-99, B-100, B-101, B-102, B-103, | |
| B-104, B-105, B-106, B-107, B-108, B-109, | |
| B-110, B-111, B-112, B-113, B-114, B-115, | |
| B-116, B-117, B-118, B-119, B-120, B-121, | |
| B-122, B-123, B-124, B-125, B-126, B-127, | |
| B-128, B-129, B-130, B-131, B-132, B-133, | |
| B-134, B-135, B-136, B-137, B-138, B-139, | |
| B-140, B-141, B-142, B-143, B-144, B-145, | |
| B-146, B-147, B-148, B-149, B-150, B-151, | |
| B-152, B-153, B-154, B-155, B-156, B-157, | |
| B-158, B-159, B-160, B-161, B-162, B-163, | |
| B-164, B-165, B-166, B-167, B-168, B-169, | |
| B-170, B-171, B-172, B-173, B-174, B-175, | |
| B-176, B-177, B-178, B-179, B-180, B-181, | |
| B-182, B-183, B-184, B-185, B-186, B-187, | |
| B-188, B-189, B-190, B-191, B-192, B-193, | |
| B-194, B-195, B-196, B-197, B-198, B-199, | |
| B-200, B-201, B-202, B-203, B-204, B-205, | |
| B-206, B-207, B-208, B-209, B-210, B-211, | |
| B-212, B-213, B-214, B-215, B-216, B-217, | |
| B-218, B-219, B-220, B-221, B-222, B-223, | |
| B-224, B-225, B-226, B-227, B-228, B-229, | |
| B-230, B-231, B-232, B-233, B-234, B-235, | |
| B-236, B-237, B-238, B-239, B-240, B-241, | |
| B-242, B-243 B-244, B-245, B-246, B-247, | |
| B-248, B-249, B-250, B-251, B-252 | |
| a compound selected from the group consisting of | frovatriptan |
| B-1, B-2, B-3, B-4, B-5, B-6, B-7, B-8, B-9, | |
| B-10, B-11, B-12, B-13, B-14, B-15, B-16, B-17, | |
| B-18, B-19, B-20, B-21, B-22, B-23, B-24, B-25, | |
| B-26, B-27, B-28, B-29, B-30, B-31, B-32, | |
| B-33, B-34, B-35, B-36, B-37, B-38, B-39, B-40, | |
| B-41, B-42, B-43, B-44, B-45, B-46, B-47, B-48, | |
| B-49, B-50, B-51, B-52, B-53, B-54, B-55, B-56, | |
| B-57, B-58, B-59, B-60, B-61, B-62, B-63, B-64, | |
| B-65, B-66, B-67, B-68, B-69, B-70, B-71, B-72, | |
| B-73, B-74, B-75, B-76, B-77, B-78, B-79, B-80, | |
| B-81, B-82, B-83, B-84, B-85, B-86, B-87, B-88, | |
| B-89, B-90, B-91, B-92, B-93, B-94, B-95, B-96, | |
| B-97, B-98, B-99, B-100, B-101, B-102, B-103, | |
| B-104, B-105, B-106, B-107, B-108, B-109, | |
| B-110, B-111, B-112, B-113, B-114, B-115, | |
| B-116, B-117, B-118, B-119, B-120, B-121, | |
| B-122, B-123, B-124, B-125, B-126, B-127, | |
| B-128, B-129, B-130, B-131, B-132, B-133, | |
| B-134, B-135, B-136, B-137, B-138, B-139, | |
| B-140, B-141, B-142, B-143, B-144, B-145, | |
| B-146, B-147, B-148, B-149, B-150, B-151, | |
| B-152, B-153, B-154, B-155, B-156, B-157, | |
| B-158, B-159, B-160, B-161, B-162, B-163, | |
| B-164, B-165, B-166, B-167, B-168, B-169, | |
| B-170, B-171, B-172, B-173, B-174, B-175, | |
| B-176, B-177, B-178, B-179, B-180, B-181, | |
| B-182, B-183, B-184, B-185, B-186, B-187, | |
| B-188, B-189, B-190, B-191, B-192, B-193, | |
| B-194, B-195, B-196, B-197, B-198, B-199, | |
| B-200, B-201, B-202, B-203, B-204, B-205, | |
| B-206, B-207, B-208, B-209, B-210, B-211, | |
| B-212, B-213, B-214, B-215, B-216, B-217, | |
| B-218, B-219, B-220, B-221, B-222, B-223, | |
| B-224, B-225, B-226, B-227, B-228, B-229, | |
| B-230, B-231, B-232, B-233, B-234, B-235, | |
| B-236, B-237, B-238, B-239, B-240, B-241, | |
| B-242, B-243 B-244, B-245, B-246, B-247, | |
| B-248, B-249, B-250, B-251, B-252 | |
| a compound selected from the group consisting of | naratriptan |
| B-1, B-2, B-3, B-4, B-5, B-6, B-7, B-8, B-9, | |
| B-10, B-11, B-12, B-13, B-14, B-15, B-16, B-17, | |
| B-18, B-19, B-20, B-21, B-22, B-23, B-24, B-25, | |
| B-26, B-27, B-28, B-29, B-30, B-31, B-32, | |
| B-33, B-34, B-35, B-36, B-37, B-38, B-39, B-40, | |
| B-41, B-42, B-43, B-44, B-45, B-46, B-47, B-48, | |
| B-49, B-50, B-51, B-52, B-53, B-54, B-55, B-56, | |
| B-57, B-58, B-59, B-60, B-61, B-62, B-63, B-64, | |
| B-65, B-66, B-67, B-68, B-69, B-70, B-71, B-72, | |
| B-73, B-74, B-75, B-76, B-77, B-78, B-79, B-80, | |
| B-81, B-82, B-83, B-84, B-85, B-86, B-87, B-88, | |
| B-89, B-90, B-91, B-92, B-93, B-94, B-95, B-96, | |
| B-97, B-98, B-99, B-100, B-101, B-102, B-103, | |
| B-104, B-105, B-106, B-107, B-108, B-109, | |
| B-110, B-111, B-112, B-113, B-114, B-115, | |
| B-116, B-117, B-118, B-119, B-120, B-121, | |
| B-122, B-123, B-124, B-125, B-126, B-127, | |
| B-128, B-129, B-130, B-131, B-132, B-133, | |
| B-134, B-135, B-136, B-137, B-138, B-139, | |
| B-140, B-141, B-142, B-143, B-144, B-145, | |
| B-146, B-147, B-148, B-149, B-150, B-151, | |
| B-152, B-153, B-154, B-155, B-156, B-157, | |
| B-158, B-159, B-160, B-161, B-162, B-163, | |
| B-164, B-165, B-166, B-167, B-168, B-169, | |
| B-170, B-171, B-172, B-173, B-174, B-175, | |
| B-176, B-177, B-178, B-179, B-180, B-181, | |
| B-182, B-183, B-184, B-185, B-186, B-187, | |
| B-188, B-189, B-190, B-191, B-192, B-193, | |
| B-194, B-195, B-196, B-197, B-198, B-199, | |
| B-200, B-201, B-202, B-203, B-204, B-205, | |
| B-206, B-207, B-208, B-209, B-210, B-211, | |
| B-212, B-213, B-214, B-215, B-216, B-217, | |
| B-218, B-219, B-220, B-221, B-222, B-223, | |
| B-224, B-225, B-226, B-227, B-228, B-229, | |
| B-230, B-231, B-232, B-233, B-234, B-235, | |
| B-236, B-237, B-238, B-239, B-240, B-241, | |
| B-242, B-243, B-244, B-245, B-246, B-247, | |
| B-248, B-249, B-250, B-251, B-252 | |
| a compound selected from the group consisting of | rizatriptan |
| B-1, B-2, B-3, B-4, B-5, B-6, B-7, B-8, B-9, | |
| B-10, B-11, B-12, B-13, B-14, B-15, B-16, B-17, | |
| B-18, B-19, B-20, B-21, B-22, B-23, B-24, B-25, | |
| B-26, B-27, B-28, B-29, B-30, B-31, B-32, | |
| B-33, B-34, B-35, B-36, B-37, B-38, B-39, B-40, | |
| B-41, B-42, B-43, B-44, B-45, B-46, B-47, B-48, | |
| B-49, B-50, B-51, B-52, B-53, B-54, B-55, B-56, | |
| B-57, B-58, B-59, B-60, B-61, B-62, B-63, B-64, | |
| B-65, B-66, B-67, B-68, B-69, B-70, B-71, B-72, | |
| B-73, B-74, B-75, B-76, B-77, B-78, B-79, B-80, | |
| B-81, B-82, B-83, B-84, B-85, B-86, B-87, B-88, | |
| B-89, B-90, B-91, B-92, B-93, B-94, B-95, B-96, | |
| B-97, B-98, B-99, B-100, B-101, B-102, B-103, | |
| B-104, B-105, B-106, B-107, B-108, B-109, | |
| B-110, B-111, B-112, B-113, B-114, B-115, | |
| B-116, B-117, B-118, B-119, B-120, B-121, | |
| B-122, B-123, B-124, B-125, B-126, B-127, | |
| B-128, B-129, B-130, B-131, B-132, B-133, | |
| B-134, B-135, B-136, B-137, B-138, B-139, | |
| B-140, B-141, B-142, B-143, B-144, B-145, | |
| B-146, B-147, B-148, B-149, B-150, B-151, | |
| B-152, B-153, B-154, B-155, B-156, B-157, | |
| B-158, B-159, B-160, B-161, B-162, B-163, | |
| B-164, B-165, B-166, B-167, B-168, B-169, | |
| B-170, B-171, B-172, B-173, B-174, B-175, | |
| B-176, B-177, B-178, B-179, B-180, B-181, | |
| B-182, B-183, B-184, B-185, B-186, B-187, | |
| B-188, B-189, B-190, B-191, B-192, B-193, | |
| B-194, B-195, B-196, B-197, B-198, B-199, | |
| B-200, B-201, B-202, B-203, B-204, B-205, | |
| B-206, B-207, B-208, B-209, B-210, B-211, | |
| B-212, B-213, B-214, B-215, B-216, B-217, | |
| B-218, B-219, B-220, B-221, B-222, B-223, | |
| B-224, B-225, B-226, B-227, B-228, B-229, | |
| B-230, B-231, B-232, B-233, B-234, B-235, | |
| B-236, B-237, B-238, B-239, B-240, B-241, | |
| B-242, B-243, B-244, B-245, B-246, B-247, | |
| B-248, B-249, B-250, B-251, B-252 | |
| a compound selected from the group consisting of | sumatriptan |
| B-1, B-2, B-3, B-4, B-5, B-6, B-7, B-8, B-9, | |
| B-10, B-11, B-12, B-13, B-14, B-15, B-16, B-17, | |
| B-18, B-19, B-20, B-21, B-22, B-23, B-24, B-25, | |
| B-26, B-27, B-28, B-29, B-30, B-31, B-32, | |
| B-33, B-34, B-35, B-36, B-37, B-38, B-39, B-40, | |
| B-41, B-42, B-43, B-44, B-45, B-46, B-47, B-48, | |
| B-49, B-50, B-51, B-52, B-53, B-54, B-55, B-56, | |
| B-57, B-58, B-59, B-60, B-61, B-62, B-63, B-64, | |
| B-65, B-66, B-67, B-68, B-69, B-70, B-71, B-72, | |
| B-73, B-74, B-75, B-76, B-77, B-78, B-79, B-80, | |
| B-81, B-82, B-83, B-84, B-85, B-86, B-87, B-88, | |
| B-89, B-90, B-91, B-92, B-93, B-94, B-95, B-96, | |
| B-97, B-98, B-99, B-100, B-101, B-102, B-103, | |
| B-104, B-105, B-106, B-107, B-108, B-109, | |
| B-110, B-111, B-112, B-113, B-114, B-115, | |
| B-116, B-117, B-118, B-119, B-120, B-121, | |
| B-122, B-123, B-124, B-125, B-126, B-127, | |
| B-128, B-129, B-130, B-131, B-132, B-133, | |
| B-134, B-135, B-136, B-137, B-138, B-139, | |
| B-140, B-141, B-142, B-143, B-144, B-145, | |
| B-146, B-147, B-148, B-149, B-150, B-151, | |
| B-152, B-153, B-154, B-155, B-156, B-157, | |
| B-158, B-159, B-160, B-161, B-162, B-163, | |
| B-164, B-165, B-166, B-167, B-168, B-169, | |
| B-170, B-171, B-172, B-173, B-174, B-175, | |
| B-176, B-177, B-178, B-179, B-180, B-181, | |
| B-182, B-183, B-184, B-185, B-186, B-187, | |
| B-188, B-189, B-190, B-191, B-192, B-193, | |
| B-194, B-195, B-196, B-197, B-198, B-199, | |
| B-200, B-201, B-202, B-203, B-204, B-205, | |
| B-206, B-207, B-208, B-209, B-210, B-211, | |
| B-212, B-213, B-214, B-215, B-216, B-217, | |
| B-218, B-219, B-220, B-221, B-222, B-223, | |
| B-224, B-225, B-226, B-227, B-228, B-229, | |
| B-230, B-231, B-232, B-233, B-234, B-235, | |
| B-236, B-237, B-238, B-239, B-240, B-241, | |
| B-242, B-243, B-244, B-245, B-246, B-247, | |
| B-248, B-249, B-250, B-251, B-252 | |
| a compound selected from the group consisting of | zolmitriptan |
| B-1, B-2, B-3, B-4, B-5, B-6, B-7, B-8, B-9, | |
| B-10, B-11, B-12, B-13, B-14, B-15, B-16, B-17, | |
| B-18, B-19, B-20, B-21, B-22, B-23, B-24, B-25, | |
| B-26, B-27, B-28, B-29, B-30, B-31, B-32, | |
| B-33, B-34, B-35, B-36, B-37, B-38, B-39, B-40, | |
| B-41, B-42, B-43, B-44, B-45, B-46, B-47, B-48, | |
| B-49, B-50, B-51, B-52, B-53, B-54, B-55, B-56, | |
| B-57, B-58, B-59, B-60, B-61, B-62, B-63, B-64, | |
| B-65, B-66, B-67, B-68, B-69, B-70, B-71, B-72, | |
| B-73, B-74, B-75, B-76, B-77, B-78, B-79, B-80, | |
| B-81, B-82, B-83, B-84, B-85, B-86, B-87, B-88, | |
| B-89, B-90, B-91, B-92, B-93, B-94, B-95, B-96, | |
| B-97, B-98, B-99, B-100, B-101, B-102, B-103, | |
| B-104, B-105, B-106, B-107, B-108, B-109, | |
| B-110, B-111, B-112, B-113, B-114, B-115, | |
| B-116, B-117, B-118, B-119, B-120, B-121, | |
| B-122, B-123, B-124, B-125, B-126, B-127, | |
| B-128, B-129, B-130, B-131, B-132, B-133, | |
| B-134, B-135, B-136, B-137, B-138, B-139, | |
| B-140, B-141, B-142, B-143, B-144, B-145, | |
| B-146, B-147, B-148, B-149, B-150, B-151, | |
| B-152, B-153, B-154, B-155, B-156, B-157, | |
| B-158, B-159, B-160, B-161, B-162, B-163, | |
| B-164, B-165, B-166, B-167, B-168, B-169, | |
| B-170, B-171, B-172, B-173, B-174, B-175, | |
| B-176, B-177, B-178, B-179, B-180, B-181, | |
| B-182, B-183, B-184, B-185, B-186, B-187, | |
| B-188, B-189, B-190, B-191, B-192, B-193, | |
| B-194, B-195, B-196, B-197, B-198, B-199, | |
| B-200, B-201, B-202, B-203, B-204, B-205, | |
| B-206, B-207, B-208, B-209, B-210, B-211, | |
| B-212, B-213, B-214, B-215, B-216, B-217, | |
| B-218, B-219, B-220, B-221, B-222, B-223, | |
| B-224, B-225, B-226, B-227, B-228, B-229, | |
| B-230, B-231, B-232, B-233, B-234, B-235, | |
| B-236, B-237, B-238, B-239, B-240, B-241, | |
| B-242, B-243, B-244, B-245, B-246, B-247, | |
| B-248, B-249, B-250, B-251, B-252 | |

By way of yet further example, Table 6 details additional suitable combinations that may be employed in the methods and compositions of the current invention. The combination may also include an isomer, a pharmaceutically acceptable salt, ester, or prodrug of any of the cyclooxygenase-2 selective inhibitors or 5-HT_{1B/1D} agonists listed in Table 6.

TABLE 6

| | 5-HT 1B/1D |
|---|---|
| Cyclooxygenase-2 Selective Inhibitor | Agonist |
| celecoxib | triptan |
| celecoxib | eletriptan |
| celecoxib | frovatriptan |
| celecoxib | naratriptan |
| celecoxib | rizatriptan |
| celecoxib | sumatriptan |
| celecoxib | zolmitriptan |
| deracoxib | triptan |
| deracoxib | eletriptan |
| deracoxib | frovatriptan |
| deracoxib | naratriptan |
| deracoxib | rizatriptan |
| deracoxib | sumatriptan |
| deracoxib | zolmitriptan |
| valdecoxib | triptan |
| valdecoxib | eletriptan |
| valdecoxib | frovatriptan |
| valdecoxib | naratriptan |
| valdecoxib | rizatriptan |
| valdecoxib | sumatriptan |
| valdecoxib | zolmitriptan |
| rofecoxib | triptan |
| rofecoxib | eletriptan |
| rofecoxib | frovatriptan |
| rofecoxib | naratriptan |
| rofecoxib | rizatriptan |
| rofecoxib | sumatriptan |
| rofecoxib | zolmitriptan |
| etoricoxib | triptan |
| etoricoxib | eletriptan |
| etoricoxib | frovatriptan |
| etoricoxib | naratriptan |
| etoricoxib | rizatriptan |
| etoricoxib | sumatriptan |
| etoricoxib | zolmitriptan |
| meloxicam | triptan |
| meloxicam | eletriptan |
| meloxicam | frovatriptan |
| meloxicam | naratriptan |
| meloxicam | rizatriptan |
| meloxicam | sumatriptan |
| meloxicam | zolmitriptan |
| parecoxib | triptan |
| parecoxib | eletriptan |
| parecoxib | frovatriptan |
| parecoxib | naratriptan |
| parecoxib | rizatriptan |
| parecoxib | sumatriptan |
| parecoxib | zolmitriptan |
| 4-(4-cyclohexyl-2-methyloxazol-5-yl)-2- | triptan |
| fluorobenzenesulfonamide | |
| 4-(4-cyclohexyl-2-methyloxazol-5-yl)-2- | eletriptan |
| fluorobenzenesulfonamide | |
| 4-(4-cyclohexyl-2-methyloxazol-5-yl)-2- | frovatriptan |
| fluorobenzenesulfonamide | |
| 4-(4-cyclohexyl-2-methyloxazol-5-yl)-2- | naratriptan |
| fluorobenzenesulfonamide | |
| 4-(4-cyclohexyl-2-methyloxazol-5-yl)-2- | rizatriptan |
| fluorobenzenesulfonamide | |
| 4-(4-cyclohexyl-2-methyloxazol-5-yl)-2- | sumatriptan |
| fluorobenzenesulfonamide | |
| 4-(4-cyclohexyl-2-methyloxazol-5-yl)-2- | zolmitriptan |
| fluorobenzenesulfonamide | |
| 2-(3,5-difluorophenyl)-3-(4- | triptan |
| (methylsulfonyl)phenyl)-2-cyclopenten-1-one | |
| 2-(3,5-difluorophenyl)-3-(4- | eletriptan |
| (methylsulfonyl)phenyl)-2-cyclopenten-1-one | |
| 2-(3,5-difluorophenyl)-3-(4- | frovatriptan |
| (methylsulfonyl)phenyl)-2-cyclopenten-1-one | |
| 2-(3,5-difluorophenyl)-3-(4- | naratriptan |
| (methylsulfonyl)phenyl)-2-cyclopenten-1-one | |
| 2-(3,5-difluorophenyl)-3-(4- | rizatriptan |
| (methylsulfonyl)phenyl)-2-cyclopenten-1-one | |
| 2-(3,5-difluorophenyl)-3-(4- | sumatriptan |
| (methylsulfonyl)phenyl)-2-cyclopenten-1-one | |
| 2-(3,5-difluorophenyl)-3-(4- | zolmitriptan |
| (methylsulfonyl)phenyl)-2-cyclopenten-1-one | |
| N-[2-(cyclohexyloxy)-4- | triptan |
| nitrophenyl]methanesulfonamide | |
| N-[2-(cyclohexyloxy)-4- | eletriptan |
| nitrophenyl]methanesulfonamide | |
| N-[2-(cyclohexyloxy)-4- | frovatriptan |
| nitrophenyl]methanesulfonamide | |
| N-[2-(cyclohexyloxy)-4- | naratriptan |
| nitrophenyl]methanesulfonamide | |
| N-[2-(cyclohexyloxy)-4- | rizatriptan |
| nitrophenyl]methanesulfonamide | |
| N-[2-(cyclohexyloxy)-4- | sumatriptan |
| nitrophenyl]methanesulfonamide | |
| N-[2-(cyclohexyloxy)-4- | zolmitriptan |
| nitrophenyl]methanesulfonamide | |
| 2-(3,4-difluorophenyl)-4-(3-hydroxy-3- | triptan |
| methylbutoxy)-5-[4-(methylsulfonyl)phenyl]- | |
| 3(2H)-pyridazinone | |
| 2-(3,4-difluorophenyl)-4-(3-hydroxy-3- | eletriptan |
| methylbutoxy)-5-[4-(methylsulfonyl)phenyl]- | |
| 3(2H)-pyridazinone | |
| 2-(3,4-difluorophenyl)-4-(3-hydroxy-3- | frovatriptan |
| methylbutoxy)-5-[4-(methylsulfonyl)phenyl]- | |
| 3(2H)-pyridazinone | |
| 2-(3,4-difluorophenyl)-4-(3-hydroxy-3- | naratriptan |
| methylbutoxy)-5-[4-(methylsulfonyl)phenyl]- | |
| 3(2H)-pyridazinone | |
| 2-(3,4-difluorophenyl)-4-(3-hydroxy-3- | rizatriptan |
| methylbutoxy)-5-[4-(methylsulfonyl)phenyl]- | |
| 3(2H)-pyridazinone | |
| 2-(3,4-difluorophenyl)-4-(3-hydroxy-3- | sumatriptan |
| methylbutoxy)-5-[4-(methylsulfonyl)phenyl]- | |
| 3(2H)-pyridazinone | |
| 2-(3,4-difluorophenyl)-4-(3-hydroxy-3- | zolmitriptan |
| methylbutoxy)-5-[4-(methylsulfonyl)phenyl]- | |
| 3(2H)-pyridazinone | |
| 2-[(2,4-dichloro-6-methylphenyl)amino]-5- | triptan |
| ethyl-benzeneacetic acid | |
| 2-[(2,4-dichloro-6-methylphenyl)amino]-5- | eletriptan |
| ethyl-benzeneacetic acid | |
| 2-[(2,4-dichloro-6-methylphenyl)amino]-5- | frovatriptan |
| ethyl-benzeneacetic acid | |
| 2-[(2,4-dichloro-6-methylphenyl)amino]-5- | naratriptan |
| ethyl-benzeneacetic acid | |
| 2-[(2,4-dichloro-6-methylphenyl)amino]-5- | rizatriptan |
| ethyl-benzeneacetic acid | |
| 2-[(2,4-dichloro-6-methylphenyl)amino]-5- | sumatriptan |
| ethyl-benzeneacetic acid | |
| 2-[(2,4-dichloro-6-methylphenyl)amino]-5- | zolmitriptan |
| ethyl-benzeneacetic acid | |
| (3Z)-3-[(4-chlorophenyl)[4- | triptan |
| (methylsulfonyl)phenyl]methylene]dihydro- | |
| 2(3H)-furanone | |
| (3Z)-3-[(4-chlorophenyl)[4- | eletriptan |
| (methylsulfonyl)phenyl]methylene]dihydro- | |
| 2(3H)-furanone | |
| (3Z)-3-[(4-chlorophenyl)[4- | frovatriptan |
| (methylsulfonyl)phenyl]methylene]dihydro- | |
| 2(3H)-furanone | |
| (3Z)-3-[(4-chlorophenyl)[4- | naratriptan |
| (methylsulfonyl)phenyl]methylene]dihydro- | |
| 2(3H)-furanone | |
| (3Z)-3-[(4-chlorophenyl)[4- | rizatriptan |
| (methylsulfonyl)phenyl]methylene]dihydro- | |
| 2(3H)-furanone | |
| (3Z)-3-[(4-chlorophenyl)[4- | sumatriptan |
| (methylsulfonyl)phenyl]methylene]dihydro- | |
| 2(3H)-furanone | |
| (3Z)-3-[(4-chlorophenyl)[4- | zolmitriptan |
| (methylsulfonyl)phenyl]methylene]dihydro- | |
| 2(3H)-furanone | |
| (S)-6,8-dichloro-2-(trifluoromethyl)-2H-1- | triptan |
| benzopyran-3-carboxylic acid | |
| (S)-6,8-dichloro-2-(trifluoromethyl)-2H-1- | eletriptan |
| benzopyran-3-carboxylic acid | |
| (S)-6,8-dichloro-2-(trifluoromethyl)-2H-1- | frovatriptan |
| benzopyran-3-carboxylic acid | |
| (S)-6,8-dichloro-2-(trifluoromethyl)-2H-1- | naratriptan |
| benzopyran-3-carboxylic acid | |
| (S)-6,8-dichloro-2-(trifluoromethyl)-2H-1- | rizatriptan |
| benzopyran-3-carboxylic acid | |
| (S)-6,8-dichloro-2-(trifluoromethyl)-2H-1- | sumatriptan |
| benzopyran-3-carboxylic acid | |
| (S)-6,8-dichloro-2-(trifluoromethyl)-2H-1- | zolmitriptan |
| benzopyran-3-carboxylic acid | |

Diagnosis of a Migraine

One aspect of the invention encompasses diagnosing a subject in need of treatment or prevention for a migraine. Usually, migraine sufferers go to their primary care doctor, who as a first step, may perform a brain scan (e.g., CT scan or MRI scan) to rule out other possible causes of headaches, such as a tumor or stroke. Generally speaking, there are no precise clinical tests that serve to make the diagnosis of migraine, tension-type, cluster, or other benign headache condition. Instead, the physician depends greatly on the headache sufferer's accuracy in describing the symptoms, the pattern of the headaches, and any suspected triggers. Migraine is typically diagnosed by determining whether some of a person's recurrent headaches meet migraine criteria. These migraine criteria include the following symptoms: no symptoms between attacks, a headache that lasts 4 to 72 hours, a headache that has at least two of the following four characteristics—location on one side of the head, pulsating pain, moderate or severe intensity that inhibits usual activity, and during headache, either nausea, vomiting, or sensitivity to light and/or noise.

Indications to be Treated

In another embodiment, preferably, the subject has had a migraine, such as a migraine without aura. The composition of the invention may be administered to a subject following migraine. The method of the invention also embraces treatment of a subject to reduce the risk of another migraine. By way of example, the type of migraine, migraine without aura, migraine with aura, ophthalmoplegic migraine, retinal migraine, abdominal migraine and a migrainous disorder, would determine the most appropriate therapy.

Combinations of triptans and non-steroidal anti-inflammatory agents such as selective cyclooxygenase-2 inhibitors may be helpful in preventing recurrence of migraine.

The composition of the invention comprising a therapeutically effective amount of a cyclooxygenase-2 selective inhibitor and a therapeutically effective amount of a 5-HT_{1B/1D} agonist may be employed to treat any type of migraine or related disorder. By way of example, such type of migraines or related disorders include but are not limited to, migraine without aura, migraine with aura, ophthalmoplegic migraine, retinal migraine, abdominal migraine and a migrainous disorder. In addition, the composition of the invention may be used for treating hypertension, drug abuse, cluster headache, chronic paroxysmal hemicrania and headache associated with vascular disorders.

Moreover, compositions of the invention may also be useful in the treatment of headache associated with meningeal irritation, including bacterial, fungal, viral, parasiti, and chemical meningitis, acquired immune deficiency syndrome (AIDS) meningovascular inflammation and subarachnoid hemorrhage. [See W. S. Lee, et al., Evidence Using Conformationally Restricted Sumatriptan Analogues, CP-122, 288 and CP-122, 638, that 5-HT_{1D} Receptors Do Not Mediate Blockade of Neurogenic Inflammation, 23^{rd} Annual Meeting of the Society for Neuroscience, Washington, D.C., Nov. 7-12, 1993, Abstract #565.6; K. Nozaki, et al. CP-93, 129, Sumatriptan, Di-hydroergotamine Block c-fos Expression Within Rat Trigeminal Nucleus Caudalis Caused by Chemical Stimulation of the Meninges, Br. J. Pharmacol. (1992), 1.06, 409; and Lee et al., Brain Research, 626, 303-305 (1993).]

Compositions of the invention may also be useful in the treatment of a large number of diseases. These include dermatological disorders, including psoriasis; eczema and atopic eczematous dermatitis; intractable itch (pruritus), including itch associated with liver cirrhosis, cancer and haemodialysis; burns and scalds; sunburn; insect bites; urticaria and sweat gland abnormalities. Other dermatological disorders include bullous penphgoid, photo dermatoses, skin blisters, adult acne, chicken pox and dermatitis herpetifunus.

Other diseases which may be treated with the compositions of the present invention are peripheral neurophathies including postherpetic neuralgia, diabetic neuropathies such as peripheral polyneuropathy and radiculopathy; causalgia and reflex sympathetic dystrophy; post-mastectomy neuralgia; post-surgical neuralgia and pain; vulvar vestibulitis; phantom limb pain; thalamic syndrome (central post-stroke pain); temporo mandibular joint syndrome; metarsalgia (Morton's neuralgia); and neurogenic pain from nerve compression caused, for example, by a prolapsed intervertebral disc or carpal and tarsal tunnel syndromes.

The above-mentioned compositions may also be useful in alleviating arthritis, including osteoarthritis, rheumatoid arthritis, systemic lupus erythrematosus, fibromyalgia, ankylosing spondilitis and tendinitis. They are also effective against gastrointestinal and urogenital diseases including cystitis, gastroesophageal reflux, gastritis, urge continence, inflammatory bowel disease and irritable bowel syndrome; they are effective in regulatory gastrointestinal tract motility.

The compositions may also be used in the treatment of headache asociated with substances or their withdrawal (e.g. drug withdrawal), tension headache, pediatric migraine and prophylaxis of migraine and post-traumatic dysautonomic cephalgia.

The compositions of the present invention may also be used for treating orofacial pain (for example toothache and pain of dental origin, earache, TMJ pain, sinus pain, myofacial pain, non-arthritic and non-musculoskeletal cervical pain), mouth ulcers, Meniere's disease and a typical facial neuralgia, and also allergic and chronic obstructive airways diseases such as rhinitis, conjunctivitis, bronchial oedema, bronchial asthma, neurological pulmonary oedema (adult respiratory disease syndrome), anaphylaxis and angioedema. The compounds are also efficacious in treating ocular pressure or glaucoma and ocular inflammation.

It is believed that the compositions of or their salts are efficacious against emesis caused by several factors not associated with migraine, including emesis induced by anaesthesia, cancer chemotherapy and by motion (seasickness, space and airsickness).

The activity of the compositions as anti-emetics may be demonstrated by the method of Tatersall et al. and Bountra et al. (European Journal of Pharmacology, 250 (1993) R5 and 249 (1993) R₃-R₄). In this method the extent to which they reduce the latency or the number of retches and/or vomits induced by emetogins in the conscious ferret compared to vehicle-treated animals is measured. It is found that the compositions are effective against emesis caused by a wide range of emetogeny, extending from local irritants to anti-cancer radiation treatment.

What is claimed is:

1. A composition comprising a 5-HT_{1B/1D} agonist or an isomer, a pharmaceutically acceptable salt, ester, or prodrug thereof and a cyclooxygenase-2 selective inhibitor or an isomer, a pharmaceutically acceptable salt, ester, or prodrug thereof, wherein the cyclooxygenase-2 selective inhibitor is a compound of the formula: 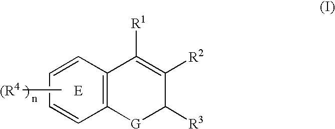
wherein
n is an integer which is 0, 1, 2, 3 or 4;
G is O, S or NR^{a};
R^{a} is alkyl;
R¹ is selected from the group consisting of H and aryl;
R² is selected from the group consisting of carboxyl, aminocarbonyl, alkylsulfonylaminocarbonyl and alkoxycarbonyl;
R³ is selected from the group consisting of haloalkyl, alkyl, aralkyl, cycloalkyl and aryl optionally substituted with one or more radicals selected from alkylthio, nitro and alkylsulfonyl; and
each R⁴ is independently selected from the group consisting of H, halo, alkyl, aralkyl, alkoxy, aryloxy, heteroaryloxy, aralkyloxy, heteroaralkyloxy, haloalkyl, haloalkoxy, alkylamino, arylamino, aralkylamino, heteroarylamino, heteroarylalkylamino, nitro, amino, aminosulfonyl, alkylaminosulfonyl, arylaminosulfonyl, heteroarylaminosulfonyl, aralkylaminosulfonyl, heteroaralkylaminosulfonyl, heterocyclosulfonyl, alkylsulfonyl, hydroxyarylcarbonyl, nitroaryl, optionally substituted aryl, optionally substituted heteroaryl, aralkylcarbonyl, heteroarylcarbonyl, arylcarbonyl, aminocarbonyl, and alkylcarbonyl; or R⁴ together with the carbon atoms to which it is attached and the remainder of ring E forms a naphthyl radical.

2. The composition of claim 1 wherein the 5-HT_{1B/1D} agonist is a triptan.

3. The composition of claim 2 wherein the triptan is selected from the group consisting of eletriptan, frovatriptan, naratriptan, rizatriptan, sumatriptan and zolmitriptan.

4. The composition of claim 1 wherein the 5-HT_{1B/1D} agonist is eletriptan.

5. A composition comprising a 5-HT_{1B/1D} agonist or an isomer, a pharmaceutically acceptable salt, ester, or prodrug thereof and a cyclooxygenase-2 selective inhibitor or an isomer, a pharmaceutically acceptable salt, ester, or prodrug thereof, wherein the cyclooxygenase-2 selective inhibitor is a compound of the formula 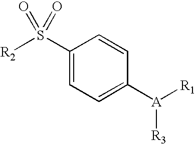
wherein
A is selected from the group consisting of partially unsaturated or unsaturated heterocyclyl and partially unsaturated or unsaturated carbocyclic rings;
R₁ is selected from the group consisting of heterocyclyl, cycloalkyl, cycloalkenyl and aryl, wherein R₁ is optionally substituted at a substitutable position with one or more radicals selected from alkyl, haloalkyl, cyano, carboxyl, alkoxycarbonyl, hydroxyl, hydroxyalkyl, haloalkoxy, amino, alkylamino, arylamino, nitro, alkoxyalkyl, alkylsulfinyl, halo, alkoxy and alkylthio;
R₂ is amino; and
R₃ is selected from the group consisting of a radical selected from H, halo, alkyl, alkenyl, alkynyl, oxo, cyano, carboxyl, cyanoalkyl, heterocyclyloxy, alkyloxy, alkylthio, alkylcarbonyl, cycloalkyl, aryl, haloalkyl, heterocyclyl, cycloalkenyl, aralkyl, heterocyclylalkyl, acyl, alkylthioalkyl, hydroxyalkyl, alkoxycarbonyl, arylcarbonyl, aralkylcarbonyl, aralkenyl, alkoxyalkyl, arylthioalkyl, aryloxyalkyl, aralkylthioalkyl, aralkoxyalkyl, alkoxyaralkoxyalkyl, alkoxycarbonylalkyl, aminocarbonyl, aminocarbonylalkyl, alkylaminocarbonyl, N-arylaminocarbonyl, N-alkyl-N-arylaminocarbonyl, alkylaminocarbonylalkyl, carboxyalkyl, alkylamino, N-arylamino, N-aralkylamino, N-alkyl-N-aralkylamino, N-alkyl-N-arylamino, aminoalkyl, alkylaminoalkyl, N-arylaminoalkyl, N-aralkylaminoalkyl, N-alkyl-N-aralkylaminoalkyl, N-alkyl-N-arylaminoalkyl, aryloxy, aralkoxy, arylthio, aralkylthio, alkylsulfinyl, alkylsulfonyl, aminosulfonyl, alkylaminosulfonyl, N-arylaminosulfonyl, arylsulfonyl, N-alkyl-N-arylaminosulfonyl; and
provided the cyclooxygenase-2 inhibitor is other than valdecoxib or celecoxib.

6. The composition of claim 5 wherein the 5-HT_{1B/1D} agonist is a triptan.

7. The composition of claim 6 wherein the triptan is selected from the group consisting of eletriptan, frovatriptan, naratriptan, rizatriptan, sumatriptan and zolmitriptan.

8. The composition of claim 5 wherein the 5-HT_{1B/1D} agonist is eletriptan.

9. A composition comprising a 5-HT_{1B/1D} agonist or an isomer, a pharmaceutically acceptable salt, ester, or prodrug thereof and a cyclooxygenase-2 selective inhibitor or an isomer, a pharmaceutically acceptable salt, ester, or prodrug thereof, wherein the cyclooxygenase-2 selective inhibitor is a compound of the formula 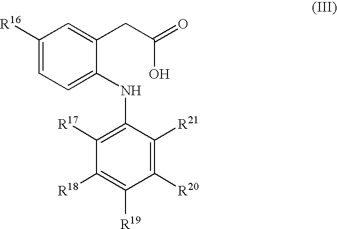
wherein:
R¹⁶ is methyl or ethyl;
R¹⁷ is chloro or fluoro;
R¹⁸ is hydrogen or fluoro;
R¹⁹ is hydrogen, fluoro, chloro, methyl, ethyl, methoxy, ethoxy or hydroxy;
R²⁰ is hydrogen or fluoro;
R²¹ is chloro, fluoro, trifluoromethyl or methyl,
provided that R¹⁷, R¹⁸, R¹⁹ and R²⁰ are not all fluoro when R¹⁶ is ethyl and R¹⁹ is H.

10. The composition of claim 9 wherein:
R¹⁶ is ethyl;
R¹⁷ and R¹⁹ are chloro;
R¹⁸ and R²⁰ are hydrogen; and
and R²¹ is methyl.

11. The composition of claim 9 wherein the 5-HT_{1B/1D} agonist is a triptan.

12. The composition of claim 11 wherein the triptan is selected from the group consisting of eletriptan, frovatriptan, naratriptan, rizatriptan, sumatriptan and zolmitriptan.

13. The composition of claim 9 wherein the 5-HT_{1B/1D} agonist is eletriptan.

14. A method for treating a migraine, the method comprising:
(a) diagnosing a subject in need of treatment for a migraine; and
(b) administering to the subject a cyclooxygenase-2 selective inhibitor that is a chromene compound, the chromene compound comprising a benzothiopyran, a dihydroquinoline or a dihydronaphthalene or an isomer, a pharmaceutically acceptable salt, ester, or prodrug of the chromene compound and a 5-HT_{1B/1D} agonist or an isomer, a pharmaceutically acceptable salt, ester, or prodrug of the 5-HT_{1B/1D} agonist.

15. The method of claim 14 wherein the cyclooxgenase-2 selective inhibitor has a selectivity ratio of COX-1 IC₅₀ to COX-2 IC₅₀ not less than about 50.

16. The method of claim 14 wherein the cyclooxgenase-2 selective inhibitor has a selectivity ratio of COX-1 IC₅₀ to COX-2 IC₅₀ not less than about 100.

17. The method of claim 14 wherein the cyclooxygenase-2 selective inhibitor is a compound having the formula 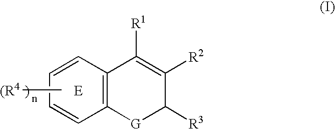
wherein:
n is an integer which is 0, 1, 2, 3 or 4;
G is O, S or NR^{a};
R^{a} is alkyl;
R¹ is selected from the group consisting of H and aryl;
R² is selected from the group consisting of carboxyl, aminocarbonyl, alkylsulfonylaminocarbonyl and alkoxycarbonyl;
R³ is selected from the group consisting of haloalkyl, alkyl, aralkyl, cycloalkyl and aryl optionally substituted with one or more radicals selected from alkylthio, nitro and alkylsulfonyl; and
each R⁴ is independently selected from the group consisting of H, halo, alkyl, aralkyl, alkoxy, aryloxy, heteroaryloxy, aralkyloxy, heteroaralkyloxy, haloalkyl, haloalkoxy, alkylamino, arylamino, aralkylamino, heteroarylamino, heteroarylalkylamino, nitro, amino, aminosulfonyl, alkylaminosulfonyl, arylaminosulfonyl, heteroarylaminosulfonyl, aralkylaminosulfonyl, heteroaralkylaminosulfonyl, heterocyclosulfonyl, alkylsulfonyl, hydroxyarylcarbonyl, nitroaryl, optionally substituted aryl, optionally substituted heteroaryl, aralkylcarbonyl, heteroarylcarbonyl, arylcarbonyl, aminocarbonyl, and alkylcarbonyl; and
R⁴ together with the carbon atoms to which it is attached and the remainder of ring E forms a naphthyl radical.

18. The method of claim 14 wherein the cyclooxgyenase-2 selective inhibitor is (S)-6,8-dichloro-2-(trifluoromethyl)-2H-1-benzopyran-3-carboxylic acid.

19. The method of claim 14 wherein the 5-HT_{1B/1D} agonist is a triptan.

20. The method of claim 19 wherein the triptan is selected from the group consisting of eletriptan, frovatriptan, naratriptan, rizatriptan, sumatriptan and zolmitriptan.

21. The method of claim 14 wherein the 5-HT_{1B/1D} agonist is eletriptan.

22. A method for treating a migraine, the method comprising:
(a) diagnosing a subject in need of treatment for a migraine; and
(b) administering to the subject a 5-HT_{1B/1D} agonist or an isomer, pharmaceutically acceptable salt, ester, or prodrug of the 5-HT_{1B/1D} agonist and a cyclooxygenase-2 selective inhibitor having the following formula: 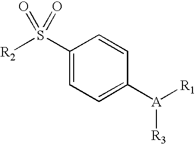
wherein
A is selected from the group consisting of partially unsaturated or unsaturated heterocyclyl and partially unsaturated or unsaturated carbocyclic rings;
R₁ is selected from the group consisting of heterocyclyl, cycloalkyl, cycloalkenyl and aryl, wherein R₁ is optionally substituted at a substitutable position with one or more radicals selected from alkyl, haloalkyl, cyano, carboxyl, alkoxycarbonyl, hydroxyl, hydroxyalkyl, haloalkoxy, amino, alkylamino, arylamino, nitro, alkoxyalkyl, alkylsulfinyl, halo, alkoxy and alkylthio;
R₂ is amino; and
R₃ is selected from the group consisting of a radical selected from H, halo, alkyl, alkenyl, alkynyl, oxo, cyano, carboxyl, cyanoalkyl, heterocyclyloxy, alkyloxy, alkylthio, alkylcarbonyl, cycloalkyl, aryl, haloalkyl, heterocyclyl, cycloalkenyl, aralkyl, heterocyclylalkyl, acyl, alkylthioalkyl, hydroxyalkyl, alkoxycarbonyl, arylcarbonyl, aralkylcarbonyl, aralkenyl, alkoxyalkyl, arylthioalkyl, aryloxyalkyl, aralkylthioalkyl, aralkoxyalkyl, alkoxyaralkoxyalkyl, alkoxycarbonylalkyl, aminocarbonyl, aminocarbonylalkyl, alkylaminocarbonyl, N-arylaminocarbonyl, N-alkyl-N-arylaminocarbonyl, alkylaminocarbonylalkyl, carboxyalkyl, alkylamino, N-arylamino, N-aralkylamino, N-alkyl-N-aralkylamino, N-alkyl-N-arylamino, aminoalkyl, alkylaminoalkyl, N-arylaminoalkyl, N-aralkylaminoalkyl, N-alkyl-N-aralkylaminoalkyl, N-alkyl-N-arylaminoalkyl, aryloxy, aralkoxy, arylthio, aralkylthio, alkylsulfinyl, alkylsulfonyl, aminosulfonyl, alkylaminosulfonyl, N-arylaminosulfonyl, arylsulfonyl, N-alkyl-N-arylaminosulfonyl; and
provided the cyclooxygenase-2 inhibitor is other than valdecoxib or celecoxib.

23. The method of claim 22 wherein the cyclooxgenase-2 selective inhibitor has a selectivity ratio of COX-1 IC₅₀ to COX-2 IC₅₀ not less than about 50.

24. The method of claim 22 wherein the cyclooxgenase-2 selective inhibitor has a selectivity ratio of COX-1 IC₅₀ to COX-2 IC₅₀ not less than about 100.

25. The method of claim 22 wherein the 5-HT_{1B/1D} agonist is a triptan.

26. The method of claim 25 wherein the triptan is selected from the group consisting of eletriptan, frovatriptan, naratriptan, rizatriptan, sumatriptan and zolmitriptan.

27. The method of claim 22 wherein the 5-HT_{1B/1D} agonist is eletriptan.

28. A method for treating a migraine, the method comprising:
(a) diagnosing a subject in need of treatment for a migraine; and
(b) administering to the subject a cyclooxygenase-2 selective inhibitor that is a phenyl acetic acid compound or an isomer, a pharmaceutically acceptable salt, ester, or prodrug of the phenyl acetic acid compound and a 5-HT_{1B/1D} agonist or an isomer, a pharmaceutically acceptable salt, ester, or prodrug of a 5-HT_{1B/1D} agonist.

29. The method of claim 28 wherein the cyclooxgenase-2 selective inhibitor has a selectivity ratio of COX-1 IC₅₀ to COX-2 IC₅₀ not less than about 50.

30. The method of claim 28 wherein the cyclooxgenase-2 selective inhibitor has a selectivity ratio of COX-1 IC₅₀ to COX-2 IC₅₀ not less than about 100.

31. The method of claim 28 wherein the cyclooxygenase-2 selective inhibitor is a compound having the formula: 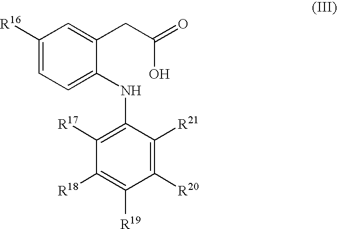
wherein:
R¹⁶ is methyl or ethyl;
R¹⁷ is chloro or fluoro;
R¹⁸ is hydrogen or fluoro;
R¹⁹ is hydrogen, fluoro, chloro, methyl, ethyl, methoxy, ethoxy or hydroxy;
R²⁰ is hydrogen or fluoro;
R²¹ is chloro, fluoro, trifluoromethyl or methyl; and
provided that R¹⁷, R¹⁸, R¹⁹ and R²⁰ are not all fluoro when R¹⁶ is ethyl and R¹⁹ is H.

32. The method of claim 31 wherein:
R¹⁶ is ethyl;
R¹⁷ and R¹⁹ are chloro;
R¹⁸ and R²⁰ are hydrogen; and
R²¹ is methyl.

33. The method of claim 28 wherein the 5-HT_{1B/1D} agonist is a triptan.

34. The method of claim 33 wherein the triptan is selected from the group consisting of eletriptan, frovatriptan, naratriptan, rizatriptan, sumatriptan and zolmitriptan.

35. The method of claim 28 wherein the 5-HT_{1B/1D} agonist is eletriptan.

36. A method for treating a migraine, the method comprising:
(a) diagnosing a subject in need of treatment for a migraine; and
(b) administering to the subject a cyclooxygenase-2 selective inhibitor selected from the group consisting of deracoxib, meloxicam, parecoxib, 4-(4-cyclohexyl-2-methyloxazol-5-yl)-2-fluorobenzenesulfonamide, 2-(3,5-difluorophenyl)-3-(4-(methylsulfonyl)phenyl)-2-cyclopenten-1-one, N-[2-(cyclohexyloxy)-4-nitrophenyl]methanesulfonamide, 2-(3,4-difluorophenyl)-4-(3-hydroxy-3-methylbutoxy)-5-[4-(methylsulfonyl)phenyl]-3(2H)-pyridazinone, 2-[(2,4-dichloro-6-methylphenyl)amino]-5-ethyl-benzeneacetic acid, (3Z)-3-[(4-chlorophenyl)[4-(methylsulfonyl)phenyl]methylene]dihydro-2(3H)-furanone, and (S)-6,8-dichloro-2-(trifluoromethyl)-2H-1-benzopyran-3-carboxylic acid; and
a 5-HT_{1B/1D} agonist selected from the group consisting of eletriptan, frovatriptan, naratriptan, rizatriptan, sumatriptan and zolmitriptan.

| Compound Number | Structural Formula |
|---|---|
| B-58 | 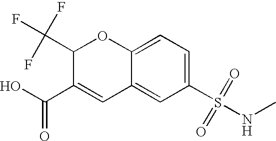 |
| | 6-[(methylamino)sulfonyl]-2-trifluoromethyl-2H-1-benzopyran-3-carboxylic acid |
| B-59 | 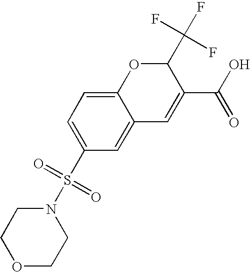 |
| | 6-[(4-morpholino)sulfonyl]-2-trifluoromethyl-2H-1-benzopyran-3-carboxylic acid |
| B-60 | 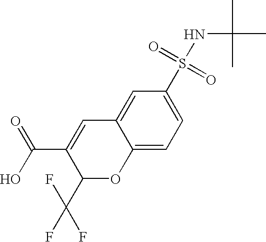 |
| | 6-[(1,1-dimethylethyl)aminosulfonyl]-2-trifluoromethyl |
| | -2H-1-benzopyran-3-carboxylic acid; |
